(19) United States
(12) Patent Application Publication
(10) Pub. No.: US 2025/0250346 A1
(43) Pub. Date: Aug. 7, 2025

(54) ANTI-SOLUBLE INTERLEUKIN-7 RECEPTOR ANTIBODY THERAPY TO TREAT AUTOIMMUNE DISEASES

(71) Applicants: BOARD OF REGENTS, THE UNIVERSITY OF TEXAS SYSTEM, Austin, TX (US); AUTOIMMUNITY BIOLOGIC SOLUTIONS, INC., Galveston, TX (US)

(72) Inventors: Mariano A. GARCIA-BLANCO, Austin, TX (US); Gaddiel GALARZA-MUNOZ, Galveston, TX (US)

(73) Assignees: BOARD OF REGENTS, THE UNIVERSITY OF TEXAS SYSTEM, Austin, TX (US); AUTOIMMUNITY BIOLOGIC SOLUTIONS, INC., Galveston, TX (US)

(21) Appl. No.: 18/856,623

(22) PCT Filed: Apr. 13, 2023
(86) PCT No.: PCT/US2023/065751

§ 371 Date: Oct. 13, 2024

Related U.S. Application Data
(60) Provisional application No. 63/330,659, filed on Apr. 13, 2022.

(51) Int. Cl. C07K 16/28; A61K 39/00; A61P 25/28; A61P 37/06

(52) U.S. Cl. CPC C07K 16/2866; A61P 25/28; A61P 37/06; A61K 2039/505

(57) ABSTRACT

The present invention provides methods and compositions for a novel antibody therapeutic against the soluble isoform of the interleukin 7 receptor (sIL7R), a potent driver of self-destructive immune responses that cause autoimmune diseases including multiple sclerosis, type I diabetes, rheumatoid arthritis, systemic lupus erythematosus as well as other autoimmune diseases. The present invention includes antibodies specific for sIL7R that inhibit sIL7R, a driver of autoimmunity, without inhibiting mIL7R, thereby reducing the severity of or preventing autoimmunity without activating immunosuppressive mechanisms.

### RELATED APPLICATIONS

This application claims priority to U.S. provisional patent application Ser. No. 63/330,659 filed on Apr. 13, 2022. The contents of the aforementioned application are incorporated herein by reference.

### FIELD OF THE INVENTION

The invention relates generally to therapies to treat autoimmune diseases, and more particularly, it relates to formulations and methods for the treating multiple sclerosis using antibodies that target soluble interleukin-7 receptor (sIL7R).

### REFERENCE TO SEQUENCE LISTING

The instant application contains a Sequence Listing which has been submitted electronically in xml format and is hereby incorporated by reference in its entirety. Said xml copy, created on Apr. 13, 2023, is named AB6-004WO-SEQID and is 152 KB in size.

### BACKGROUND

Multiple sclerosis (MS) is the most common neurological disease of early adulthood and creates a high physical, emotional and financial toll on patients afflicted with this disease, their relatives and society. MS is an autoimmune disorder of the central nervous system that results from a destructive immune-mediated attack against neuronal myelin sheaths that cause demyelination and neuronal death. Patients diagnosed with MS can suffer from debilitating cognitive, sensory, and motor impairments that often lead to significant disability. MS is a devastating diagnosis for which there is no cure. Although significant progress has been made in recent years in the development of effective MS treatments, the current approved drugs still rely on immunomodulatory mechanisms that can result in severe or even fatal adverse side-effects. These include leukopenia and immunosuppression, the latter of which can lead to an increased risk of infections and cancer.

Current MS therapeutic treatments that have been approved for patient use, including Tysabri, Tecfidera, Gilenya and Ocrevus, have been found to increase the risk of a viral infection that causes Progressive Multifocal Leukoencephalopathy (PML), an inflammation of brain white matter that is typically lethal. Other detrimental side-effects from the use of the aforementioned therapeutic treatments include liver and kidney malfunction and reduced vaccination immunity.

An underappreciated limitation of current MS therapeutics is that patients are commonly diagnosed at ages between 20-40 and must take the MS therapeutics for the remainder of their lives, usually decades, and the long-term adverse effects of these drugs are largely unknown. Supporting this, patients are often forced to switch treatments due to adverse effects.

While it is understood that MS is a complex disease with clear genetic and environmental etiological components, the precise mechanism that causes MS remains unclear. It is widely accepted that MS develops in individuals that are predisposed to the disease by their genetic makeup, and that it occurs following exposure to an environmental trigger, such as an infectious agent. Because there are a multitude of etiological genetic factors that can predispose an individual to develop MS, it has been difficult to develop a safe and effective therapy. In part, this has been driven due to poor knowledge regarding the molecular mechanisms underlying these etiologies.

It has been found that the interleukin 7 receptor alpha chain (IL7R) is strongly and reproducibly associated with an increased MS risk. IL7R encodes the alpha chain of the interleukin 7 receptor (mIL7R), which together with the common cytokine receptor gamma chain (IL2RG) forms a receptor complex for IL7 on the surface of T cells. Signaling of IL7 through this receptor complex has been found to provide signals crucial for the survival, proliferation and maintenance of T cells.

It is believed that mIL7R is the key regulatory molecule in the IL7 signaling pathway, and its expression is dynamically regulated throughout lymphopoiesis and upon T cell activation. Not surprisingly, it is also believed that mIL7R plays a central role in maintaining T cell homeostasis. This feature has been seen through experimental work using IL7R knockout in mice and loss-of-function mutations in humans; both of which were shown to cause lymphopenia and severe immunodeficiencies. Accordingly, it is believed that MS therapies, including the use of antibodies (e.g., monoclonal antibodies or mAbs) that inhibit mIL7R function can result in immunosuppression, an increased risk of cancer and infections with associated morbidity and mortality.

The IL7R gene produces two protein isoforms, namely the membrane-bound IL7R (mIL7R) and a secreted form named soluble IL7R (sIL7R), and the expression of these proteins is determined by alternative splicing of exon 6. This critical exon encodes the single transmembrane domain of mIL7R and transcripts that include this exon encode the canonical mIL7R, whereas transcripts that exclude the exon encode the secreted sIL7R. We have previously determined that SNP rs6897932 (C/T) located in IL7R exon 6 influences splicing of exon 6 and is strongly associated with increased MS risk (see FIG. 1). Specifically, we determined that the risk ‘C’ allele of rs6897932 increases exclusion (skipping) of exon 6, thereby enhancing the fraction of RNAs that encode sIL7R, leading to elevated levels of circulating SIL7R. In fact, while this genetic variant causes a moderate change in splicing of the exon and a negligible reduction in mIL7R, it significantly increases sIL7R secretion. This increase in sIL7R secretion has pathological consequences. Particularly as sIL7R has been shown to exacerbate the severity of Experimental Autoimmune Encephalomyelitis (EAE) in a mouse model of MS.

Through our work, we have determined that it appears the RNA helicase DDX39B is a splicing factor critical for inclusion of exon 6 in IL7R pre-mRNAs and is a potential risk factor for MS. More particularly, we determined that the SNP rs2523506 in the DDX39B gene is associated with low DDX39B protein levels and increased risk of MS. Moreover, we determined that an epistatic interaction between this SNP and rs6897932 in IL7R that results in increased skipping of IL7R exon 6, which can result in a 3-fold increase in MS risk. Importantly, it has been found that DDX39B and IL7R have been associated with multiple autoimmune diseases such as MS, type 1 diabetes, rheumatoid arthritis and systemic lupus erythematosus. It has also been found that patients suffering from these diseases have a tendency to exhibit high levels of sIL7R that correlate with the autoimmune disease activity. This suggests that sIL7R up-regulation via skipping of IL7R exon 6 might be a mechanism that triggers autoimmunity and its associated diseases, including MS.

Accordingly, there is a clear unmet need for the development of an effective therapy to treat MS that is safer for patients and does not result in significant immunosuppression. Including development of an effective therapy better tailored to specific etiologies.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

Embodiments include a pharmaceutical composition that includes anti-sIL7R antibodies that bind to and neutralize sIL7R (soluble isoform of the interleukin 7 receptor) without inhibiting the alpha chain of the interleukin 7 receptor (mIL7R). In aspects, the antibodies reduce the activity of sIL7R and/or reduce detectable levels.

In embodiments, the present disclosure solves the problems described above by providing a method for treating an autoimmune disease in a subject in need thereof. The methods can include administering a therapeutically effective amount of a composition comprising an antibody (or antagonist) to neutralize/inhibit activity of sIL7R.

In another aspect, the method includes co-administering a therapeutically effective amount the antibody to a subject with a second medicament. The second medicament can be an immunomodulatory agent such as a corticosteroid. The second medicament can be a biologic (e.g., a second antibody formulation).

Accordingly, embodiments include methods and compositions of anti-sIL7R antibodies that bind to and neutralize sIL7R without inhibiting mIL7R. The antibodies can target one or two regions of sIL7R that are different from mIL7R. In an embodiment, these regions are the C terminal tail that is specific to sIL7R, and the IL7 binding pocket in the extracellular domain.

The compositions described herein can include monoclonal antibodies and antibody fragments that neutralize sIL7R specifically without inhibiting mIL7R. By not inhibiting mIL7R, the antibody/antibodies reduce the likelihood of immunosuppression occurring.

Neutralizing and/or reducing the amount of sIL7R an lead to a reduction in the severity or elimination of the symptoms associated with MS.

The antibodies against sIL7R constitute an effective and safer agent for targeting autoimmune diseases. The antibodies have broad applicability in autoimmunity and can be used to treat other autoimmune diseases such as multiple sclerosis (MS), type 1 diabetes, rheumatoid arthritis, systemic lupus erythematosus, Sjøgren's syndrome, Crohn's disease, ulcerative colitis, celiac disease, psoriasis, atopic dermatitis, ankylosing spondylitis, primary biliary cirrhosis, autoimmune encephalomyelitis, autoimmune hepatitis, nephritis, lupus nephritis, graves' disease, myasthenia gravis, myelin oligodendrocyte glycoprotein antibody disorder, psoriatic arthritis, scleroderma and other diseases and afflictions known in the art.

In embodiments, the antibodies described herein recognize a specific portion of sIL7R that is not shared with mIL7R. The portion can be, for example, the sIL7R-specific C-terminal tail (encoded by exon 7 in transcripts that skip exon 6), and/or the region of sIL7R encoded by the junction between exons 5 and 7 in transcripts that skip exon 6. Both of these regions are specific to sIL7R and are predicted to prevent binding of IL7 to sIL7R but not to mIL7R.

Embodiments include methods of determining the likelihood that a subject has an autoimmune disease (such as MS) or a predisposition based on the presence of elevated levels of sIL7R (e.g., elevated expression levels). An immuno-based assay can be used to assay the elevated levels. The method can include administering a therapeutic or immunomodulating agent to the subject to treat/prevent/ameliorate the autoimmune disease (such as MS). In aspects, levels of SNP rs6897932 (C/T) are used to demonstrate an increased MS risk. In aspects, an increased risk is demonstrated by the presence of ‘C’ allele of rs6897932 of exon 6.

### BRIEF DESCRIPTION OF THE FIGURES

FIG. 1 is a depiction of a novel sIL7R autoimmunity pathway. sIL7R is an etiological factor that promotes autoreactivity in MS. It is up-regulated in MS by a defect in splicing of IL7R exon 6 caused by the MS-associated SNP rs6897932 (C*/T) in exon 6. In addition to MS, sIL7R is upregulated in other autoimmune diseases, including type 1 diabetes, rheumatoid arthritis, systemic lupus erythematosus and others. To treat MS and autoimmune diseases where sIL7R is active, our approach includes use of antibodies that neutralize sIL7R function without inhibiting mIL7R (anti-sIL7R antibodies).

FIG. 2A shows the amino acid sequence for human mIL7R with the sequence corresponding to the different domains of sIL7R.

FIG. 2B shows the amino acid sequence for human sIL7R with the sequence corresponding to the different domains of mIL7R.

FIGS. 3A, 3B, 3C and 3D depict the arrangement of the membrane-bound receptor complex for IL7 composed of mIL7R and the common cytokine receptor gamma chain (IL2RG) with and without IL7. FIGS. 3A and 3C illustrate the arrangement of the membrane-bound receptor complex for IL7 composed of mIL7R and the common cytokine receptor gamma chain (IL2RG) with (3A) and without (3C) IL7. mIL7R is shown in blue, IL2RG is shown in green, and IL7 is shown in yellow. FIGS. 3B and 3D illustrate the structure of sIL7R with (3B) and without (3D) IL7. sIL7R is shown in blue with the C-terminal tail in red, and IL7 in yellow.

FIG. 4 shows the amino acid sequence alignment of human mIL7R and sIL7R illustrating the sIL7R-specific C-terminal tail.

FIG. 5 shows an amino acid sequence of human sIL7R illustrating the IL7 binding region, the sIL7R-specific C-terminal tail and the junction between them.

FIG. 6A illustrates a screen of anti-sIL7R mAb clones binding to sIL7R by ELISA assay using hybridoma cell supernatants. Supernatants (100 μl) from 103 hybridoma cells were screened for binding to sIL7R in ELISA assays.

FIG. 6B illustrates a screen of anti-sIL7R mAb clones binding to sIL7R by ELISA assay using purified antibodies. Purified mAbs from the 35 positive clones were used as detection antibody at 10 μg/ml in ELISA assays. A validated anti-IL7R mAb (clone 40131; red diamond) was used as positive control. 27 clones bound to sIL7R at an arbitrary cutoff of 2 times over background, 14 of which bound to sIL7R at an arbitrary cutoff of 5 times over background.

FIG. 6C illustrates an assessment of anti-sIL7R mAb clones binding to mIL7R by extracellular staining of human primary CD4⁺ T cells using purified antibodies. Purified mAbs (10 μg/ml) of the 35 positive clones were tested for binding to mIL7R by extracellular staining of intact human primary T cells and mean fluorescence intensity (MFI) was quantified by flow cytometry. Whereas the positive control bound to mIL7R in the surface of the stained T cells, none of the 35 mAb clones did.

FIG. 6D displays sIL7R specificity (sIL7R/mIL7R binding ratio).

FIG. 7 illustrates sIL7R dose-response binding curves of top α-sIL7R mAb clones. Dose-response curves of the top eight clones (05, 08, 14, 18, 22, 24, 96 & 102) binding to sIL7R were determined by ELISA using purified mAbs as detection antibody at the indicated concentrations. All the antibodies bound to sIL7R with high affinity (low nM range) and their apparent EC₅₀ values were as follow (in ascending order): 05 (4 nM), 24 (13 nM), 08 (30 nM), 96 (40 nM), 102 (53 nM) 14 (60 nM), 22 (71 nM) and 18 (80 nM).

FIG. 8 illustrates an inhibition of sIL7R activity by lead α-sIL7R mAb clones. The two clones with the lowest EC₅₀, clones 05 (ABS-2005) and 102 (ABS-2102), were tested for their ability to inhibit sIL7R. Inhibition of sIL7R was assessed by quantification of the IL7-induced gene BCL2, whose expression is enhanced by sIL7R. Human primary CD4⁺ T cells were cultured for 2 days to let secreted sIL7R accumulate in the media. The cells were then incubated with α-sIL7R mAbs for 3 hours, followed by treatment with 1 ng/ml of human recombinant IL7 for 24 hours, and quantification of BCL2 by qRT-PCR.

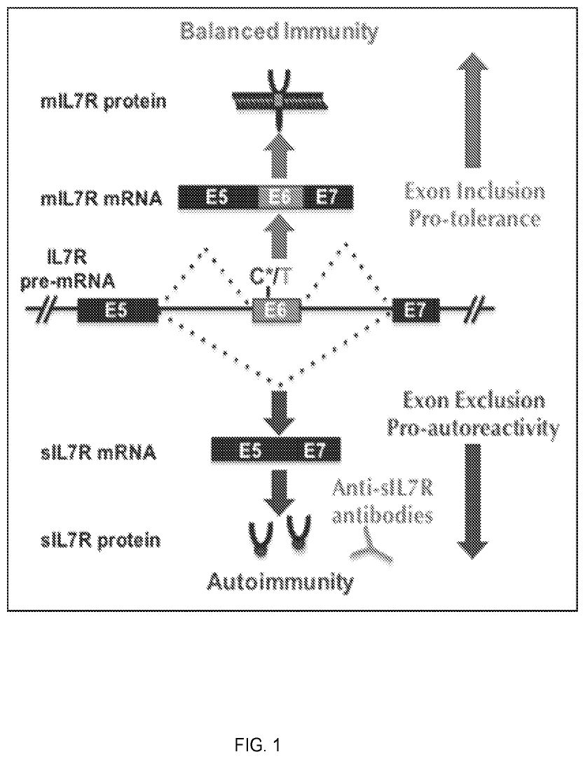

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Amino acid mimetics” refer to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “single nucleotide polymorphism” or “SNP” refers to a genetic variation in which a single nucleotide is replaced by another nucleotide within a strand of DNA.

The term “natural” or “naturally occurring” as used herein refers to what is found in nature.

The terms “optional” or “optionally” mean that the feature or structure may or may not be present, or that an event or circumstance may or may not occur, and that the description includes instances where a particular feature or structure is present and instances where the particular feature or structure is absent, or instances where the event or circumstance occurs and instances where the event or circumstance does not occur.

The term “pH adjusting agent” as used herein refers to an agent that raises or lowers the pH of a substance, including a solution or a semi-solid, such as a meat analog.

The terms “polypeptide,” “peptide” and “protein” are used interchangeably herein to refer to a polymer of amino acid residues. The terms apply to amino acid polymers in which one or more amino acid residues are an artificial chemical mimetic of a corresponding and naturally occurring amino acid, as well as to naturally occurring amino acid polymers and non-naturally occurring amino acid polymer. Methods for obtaining (e.g., producing, isolating, purifying, synthesizing, and recombinantly manufacturing) polypeptides are well known to one of ordinary skill in the art.

The term “antibody” as used herein is intended to include antibodies, immunoglobulin chains, monoclonal antibodies, polyclonal antibodies, chimeric antibodies, and antibody variants containing one or more characteristic structures of an antibody. Characteristic structures of an antibody include antibody fragments, antibody hinge regions, the Fc region, a full-length heavy chain, a full-length light chain, a variable region, a constant region, a CDR (1, 2, or 3), etc. The antibody structures may be glycosylated or free from glycosylation.

A variant of a characteristic structure of an antibody is an antibody or antibody fragment including Fc fragments containing mutations called “Knob-and Hole” as disclosed in U.S. Pat. No. 7,642,228, the entire contents of which is hereby expressly incorporated by reference. A “knob-in-hole” structure uses a “protuberance-into-cavity” strategy which serves to engineer an interface between a first and second polypeptide for hetero-oligomerization. The preferred interface includes at least a part of the CH3 domain of an antibody constant domain. “Protuberances” are constructed by replacing small amino acid side chains from the interface of the first polypeptide with larger side chains (e.g., tyrosine or tryptophan). Compensatory “cavities” of identical or similar size to the protuberances are optionally created on the interface of the second polypeptide by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

The antibody may be from recombinant sources and/or produced in transgenic animals according to art recognized methods. Portions of the bi-specific binding protein and/or the fusion protein may be expressed separately and assembled post-expression or may be simultaneously expressed by one or more nucleotide sequences encoding the bi-specific binding protein or the fusion protein.

In an embodiment, an antibody is an antibody fragment. In a further embodiment, an antibody is a binding protein. In another embodiment, an antibody binds specifically, specifically binds or has specific binding to its target, including sIL7R. In a further embodiment, an antibody, including an antibody that binds to and, in an embodiment, neutralizes sIL7R, is a modified antibody or antibody fragment that binds with greater affinity than an unmodified antibody or antibody fragment. In an embodiment an antibody that that binds to and in an embodiment, neutralizes sIL7R is multi-specific, including bispecific or can be multi-valent, including bi-valent and/or tri-valent.

It is contemplated that when the synthetic protein contains structures from immunoglobulin (Ig) molecules, that the synthetic protein be multi-valent. That is, it has binding domains for at least two antigens. In one embodiment, the synthetic protein is bi-valent. In one embodiment, the synthetic protein is tri-valent, for instance, it has three binding domains and can bind three antigen molecules simultaneously.

The present invention further relates to a polypeptide including at least one further domain, said domains being linked by covalent or non-covalent bonds. The linkage can be based on genetic fusion according to the methods known in the art and described above or can be performed by, e.g., chemical cross-linking as described in, e.g., WO 94/04686. The additional domain present in the polypeptide of the invention may preferably be linked by a flexible linker, advantageously a polypeptide linker to one of the binding site domains wherein said polypeptide linker includes plural, hydrophilic, peptide-bonded amino acids of a length sufficient to span the distance between the C-terminal end of one of said domains and the N-terminal end of the other of said domains when said polypeptide assumes a conformation suitable for binding when disposed in aqueous solution. Preferably the linker includes 1 to 15 amino acids, although polypeptide linkers of more than 15 amino acids may work as well. In a preferred embodiment, the linker includes 1 to 5 amino acid residues. In one aspect, the linker may have 1, 2, 3, 4, 5, 6, 7, 8, 9, 10, 11, 12, 13, 14, 15, or more amino acid residues. The polypeptide of the invention may further comprise a cleavable linker or cleavage site for proteinases, such as enterokinase. Said polypeptide linker may advantageously comprise the amino acid sequence Gly Gly Gly Gly Ser (SEQ ID NO: 1).

The term “analogue” as used herein denotes a peptide, polypeptide, or protein sequence which differs from a reference peptide, polypeptide, or protein sequence. Such differences may be the addition, deletion, or substitution of amino acids, phosphorylation, sulfation, acrylation, glycosylation, methylation, farnesylation, acetylation, amidation, and the like, the use of non-natural amino acid structures, or other such modifications as known in the art.

As to “conservatively modified variants” of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

Compositions can include antibodies with amino acid substitutions that do not generally alter the activity of the proteins or peptides (see, e.g., H. Neurath, R. L. Hill, The Proteins, Academic Press, New York, 1979). In one embodiment, these substitutions are “conservative” amino acid substitutions. The most commonly occurring substitutions are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

An amino acid and derivatives thereof can include cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate, glutamine, glycine, proline, serine, tyrosine, ornithine, carnosine, citrulline, carnitine, ornithine, theanine, and taurine.

The term “binding protein” as used herein refers to proteins that specifically bind to another substance, such as a surface target of an infectious agent or CD3. In an embodiment, binding proteins are antibodies or antibody fragments.

The term “binding proteins of the invention” as used herein includes antibodies or antibody fragments of the invention.

As used herein, the terms “bind specifically,” “specifically bind” and “specific binding” are understood to mean that the antibody has a binding affinity for a particular antigen (more specifically, the surface target of an infectious agent, or CD3) of at least about 10⁶ M⁻¹, or at least about 10⁷ M⁻¹, or at least about 10⁸ M⁻¹, or at least about 10⁹ M⁻¹ or at least about 10¹⁰ M⁻¹.

When a binding affinity for a first antigen is described as “stronger” than the binding affinity for the second antigen, it is contemplated that the binding affinity of the binding protein of the invention is greater for the first antigen than for the second antigen. In one aspect, the binding affinity for the first antigen is at least 1.5 times as great, at least 2 times as great, at least 3 times as great, at least 4 times as great, at least five times as great, at least six times as great, at least seven times as great, at least eight times as great, at least nine times as great, at least 10 times as great, at least 12 times as great, at least 15 times as great, at least 20 times as great, 25 times as great, at least 35 times as great, at least 50 times as great, at least 75 times as great or at least 100 times as great.

By “multi-specific” it is contemplated that the synthetic protein specifically binds at least two different antigens. In one embodiment, the synthetic protein is bi-specific, binding two different antigens. In another embodiment, the synthetic protein specifically binds at least three different antigens.

The term “antibody or antibody fragment” as used herein includes at least one light chain complementarity determining region (CDR) of the invention and/or at least one heavy chain complementarity determining region of the invention. In an aspect, the antibody or antibody fragment includes the light chain CDR sequences and/or the heavy chain CDR sequences or functional variants of the sequences so that the antibody or antibody fragment can bind to the virus, viral protein, bacterial cell, or fungal cell without substantially binding to normal cells.

The term “antibody fragment” as used herein is further intended to include Fab, Fab′, F(ab′)₂, scFv, dsFv, ds-scFv, dimers, minibodies, nanobodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, nanobodies, diabodies, bispecific antibody fragments and other fragments can also be synthesized by recombinant techniques. In another aspect a full-length antibody including both a heavy and light chain may be bound to another feature, such as a CD3 binding region (as in an aspect of the first embodiment) or a chemokine, cytokine, a chemokine, an interleukin, a growth factor or fragments thereof (as in an aspect of the second embodiment).

The phrase “biologically compatible form suitable for administration in vivo” refers to a form of the substance to be administered in which any toxic effects are outweighed by the therapeutic effects.

A “conservative amino acid substitution”, as used herein, is one in which one amino acid residue is replaced with another amino acid residue without abolishing the protein's desired properties.

The term “derivative of a peptide” refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “heavy chain complementarity determining region” as used herein refers to regions of hypervariability within the heavy chain variable region of an antibody molecule. The heavy chain variable region has three complementarity determining regions termed heavy chain complementarity determining region 1, heavy chain complementarity determining region 2 and heavy chain complementarity determining region 3 from the amino terminus to carboxy terminus.

The term “heavy chain variable region” as used herein refers to the variable region of a heavy chain.

The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e., sequences located at the 5′ and 3′ ends of the nucleic acid) from which the nucleic acid is derived. The term “nucleic acid” is intended to include DNA and RNA and can be either double stranded or single stranded.

The term “isolated proteins” refers to a protein substantially free of cellular material or culture medium when produced by recombinant DNA techniques, or chemical precursors or other chemicals when chemically synthesized.

As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

The term “light chain complementarity determining region” as used herein refers to regions of hypervariability within the light chain variable region of an antibody molecule. Light chain variable regions have three complementarity determining regions termed light chain complementarity determining region 1, light chain complementarity determining region 2 and light chain complementarity determining region 3 from the amino terminus to the carboxy terminus.

The term “light chain variable region” as used herein refers to the variable region of a light chain.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The term “sample” as used herein refers to any fluid, cell or tissue sample from a subject which can be assayed for an infectious agent. That is, any biological tissue or excreted material from an organism. In one embodiment, the subject is a plant, dirt, biological, tissue or water sample. Examples of samples include blood, mucous, sperm, urine, organ tissues, finger and toe nails, hair, skin cells, stool, milk, tears, bile, marrow and tissue samples from organs including the lungs, liver, kidney, heart, bladder, esophagus, stomach, breasts, prostate, arteries, veins, lymph nodes, lymph ducts, tongue, salivary gland, large intestine, small intestine, pituitary gland, pineal gland, thymus, thyroid gland, parathyroid gland, adrenal gland, ovary, oviduct, uterus, vagina, vulva, penis, testis, spleen, brain, spinal cord, nose, pharynx, larynx, trachea, eye, ear, and bowel.

The term “subject” as used herein refers to any member of the animal kingdom, preferably a mammal, more preferably a human being. In a preferred embodiment, the subject is suspected of being infected by or is infected by an infectious agent. In another embodiment, the organism is an animal. In one embodiment, the animal is a bird, a mammal, or a fish. In one aspect the mammal is a dog, cat, horse, goat, sheep, rat, mouse, hamster, rabbit, monkey, ape, or human.

The term “sequence identity” as used herein refers to the percentage of sequence identity between two polypeptide sequences. In order to determine the percentage of identity between two polypeptide sequences, the amino acid sequences of such two sequences are aligned, preferably using the Clustal W algorithm (Thompson, J D, Higgins D G, Gibson T J, 1994, Nucleic Acids Res. 22 (22): 4673-4680), together with BLOSUM 62 scoring matrix (Henikoff S, and Henikoff J. G., 1992, Proc. Natl. Acad. Sci. USA 89:10915-10919) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol., 1970, 48:443), as revised by Smith and Waterman (Adv. Appl. Math., 1981, 2:482) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12:387) BLASTP, BLASTN and FASTA (Altschul et al., J. Molec. Biol., 1990:215: 403). In one aspect the present binding proteins and fusion proteins have at least 70%, at least 75%, at least 80%, at least 85%, at least 87%, at least 90%, at least 91%, at least 92%, at least 93%, at least 94% at least 95%, at least 96%, at least 97%, at least 98%, at least 99% or 100% sequence identity with another sequence, either on a local or a full-length basis. If on a local basis, the locality is determined by the epitope, the CDR, the binding region, or a specifically identified motif of the original sequence. In one aspect the locality is at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 175, at least 200, at least 225, at least 250, at least 275, at least 300, at least 350, at least 400, at least 450 or at least 500 nucleic acids or amino acids of the other sequence.

The term “variant” as used herein includes modifications or chemical equivalents of the amino acid and nucleotide sequences of the present invention that perform substantially the same function as the proteins or nucleic acid molecules of the invention in substantially the same way. For example, variants of proteins of the invention include, without limitation, conservative amino acid substitutions. Variants of proteins of the invention also include additions and deletions to the proteins of the invention. In addition, variant peptides and variant nucleotide sequences include analogs and derivatives thereof. Variants may have up to 10 amino acid residue substitutions, deletions or additions, up to 9 amino acid residue substitutions, deletions or additions, up to 8 amino acid residue substitutions, deletions or additions, up to 7 amino acid residue substitutions, deletions or additions, up to 6 amino acid residue substitutions, deletions or additions, up to 5 amino acid residue substitutions, deletions or additions, up to 4 amino acid residue substitutions, deletions or additions, up to 3 amino acid residue substitutions, deletions or additions, or up to 2 amino acid residue substitutions, deletions or additions. Variants may have at least 1 amino acid residue substitutions, deletions or additions. In one aspect, the variants have at least 2 amino acid residue substitutions, deletions or additions, at least 3 amino acid residue substitutions, deletions or additions, at least 4 amino acid residue substitutions, deletions or additions, at least 5 amino acid residue substitutions, deletions or additions, at least 6 amino acid residue substitutions, deletions or additions, at least 7 amino acid residue substitutions, deletions or additions, at least 8 amino acid residue substitutions, deletions or additions, at least 9 amino acid residue substitutions, deletions or additions, or at least 10 amino acid residue substitutions, deletions or additions.

As used herein, the term “sustained release” refers to the release of a therapeutic disclosed herein over a period of about seven days or more. As used herein, the term “extended release” refers to the release of a therapeutic, including an antibody disclosed herein over a period of time of less than about seven days.

As used herein, the term “pharmacologically-acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.”

The term “autoimmune disease” refers to a condition arising from an abnormal immune response to a functioning body part. At least 80 types of autoimmune diseases have been identified, with some evidence suggesting that there may be more than 100 types. Nearly any body part can be involved. Common symptoms can be diverse and transient, ranging from mild to severe, and generally include low grade fever and feeling tired. The cause is unknown. Some autoimmune diseases such as lupus run in families, and certain cases may be triggered by infections or other environmental factors. Some common diseases that are generally considered autoimmune include multiple sclerosis (MS), type 1 diabetes, rheumatoid arthritis, systemic lupus erythematosus, Sjøgren's syndrome, Crohn's disease, ulcerative colitis, celiac disease, psoriasis, atopic dermatitis, ankylosing spondylitis, primary biliary cirrhosis, autoimmune encephalomyelitis, autoimmune hepatitis, nephritis, lupus nephritis, graves' disease, myasthenia gravis, myelin oligodendrocyte glycoprotein antibody disorder, psoriatic arthritis, scleroderma. The diagnosis can be difficult to determine. Treatment depends on the type and severity of the condition. Nonsteroidal anti-inflammatory drugs (NSAIDs) and immunosuppressants are often used. Intravenous immunoglobulin may also occasionally be used. While treatment usually improves symptoms, they do not typically cure the disease.

The term “immunosuppression” refers to a reduction of the activation or efficacy of the immune system. Some portions of the immune system itself have immunosuppressive effects on other parts of the immune system, and immunosuppression may occur as an adverse reaction to treatment of other conditions. In general, deliberately induced immunosuppression is performed to prevent the body from rejecting an organ transplant. Additionally, it is used for treating graft-versus-host disease after a bone marrow transplant, or for the treatment of autoimmune diseases such as systemic lupus erythematosus, rheumatoid arthritis, Sjögren's syndrome, or Crohn's disease. This is typically done using medications, but may involve surgery (splenectomy), plasmapheresis, or radiation.

An “immunomodulating agent” or “immunomodulator” generally refers to a substance that affects the functioning of the immune system. Immunomodulators are derived from a diverse array of recombinant, synthetic and natural preparations such as interleukins, cytokines, chemokines, and immunomodulatory imide drugs (IMiDs). Specific immunomodulating agents, such as monoclonal antibodies, cytokines, and vaccines, affect specific parts of the immune system. Nonspecific immunomodulating agents, such as BCG and levamisole, affect the immune system in a general way. Other immunomodulating agents include steroids, Cyclosporine A, tacrolimus (either individually, or combined with rapamycin) or azathioprine and T cell inhibitors.

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, NY 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, NY 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor NY, 2013); Köhler and Milstein, Derivation of specific antibody-producing tissue culture and tumor lines by cell fusion, Eur. J. Immunol. 1976 Jul. 6 (7): 511-9; Queen and Selick, Humanized immunoglobulins, U.S. Pat. No. 5,585,089 (1996 December); and Riechmann et al., Reshaping human antibodies for therapy, Nature 1988 Mar. 24, 332 (6162): 323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Conventional methods of treating autoimmune diseases generally involve administering immunosuppressive medications or immunosuppressants. Among other side-effects, immunosuppressive drugs can cause immunodeficiency, which can increase susceptibility to opportunistic infection and decrease cancer immunosurveillance. Other common side effects can include loss of appetite, nausea, vomiting, increased hair growth, and hand trembling. Aspects of the invention include targeted immunomodulatory agents that do not suppress activities of the immune system.

To develop a Multiple Sclerosis (MS) therapy that avoids immunosuppression, the present invention provides antibodies, including monoclonal antibodies that specifically neutralize sIL7R, or reduce its levels, without inhibiting mIL7R. By not inhibiting mIL7R, the antibody/antibodies reduce the likelihood of immunosuppression occurring. Additionally, the antibodies, including monoclonal antibodies, are capable of neutralizing and/or reducing the amount of sIL7R. This leads to a reduction in the severity or elimination of the symptoms associated with MS. The antibodies against sIL7R constitute an effective and safer agent for targeting autoimmune diseases such as MS. These anti-sIL7R antibodies have broad applicability in autoimmunity because they are likely applicable to other autoimmune diseases where sIL7R is of known importance, like type 1 diabetes, rheumatoid arthritis, systemic lupus erythematosus, and other diseases and afflictions known in the art.

Similarly, the antibodies described herein be used for diagnostic tests. For example, tjeu antibodies enable the identification and treatment of a patient population with high sIL7R expression that may be susceptible to MS or other autoimmune diseases. This highly characterizable patient population is predicted to be up to 60% of relapsing-remitting MS patients based on the frequency of the MS risk SNPs (single nucleotide polymorphisms) that increase sIL7R expression (rs6897932 in IL7R and rs2523506 in DDX39B).

In further embodiments, provided herein are methods for treating an autoimmune disease in a subject in need thereof. The methods can include determining the likelihood that a subject has an autoimmune disease (such as MS) or a predisposition based on the presence of elevated levels of sIL7R (e.g., elevated expression levels). An immuno-based assay can be used to assay the elevated levels. The method can include administering a therapeutic or immunomodulating agent to the subject to treat/prevent/ameliorate the autoimmune disease (such as MS).

Accordingly, embodiments include methods and compositions of anti-sIL7R antibodies that bind to and neutralize sIL7R, or reduce its levels, without inhibiting mIL7R. The antibodies can target one or two regions of sIL7R that are different from mIL7R. In an embodiment, these regions are the C terminal tail that is specific to sIL7R, and the IL7 binding pocket in the extracellular domain.

In an embodiment, anti-sIL7R antibodies are used to treat an autoimmune disease. Among the autoimmune diseases include, but are not limited to, multiple sclerosis, type 1 diabetes, rheumatoid arthritis, systemic lupus erythematosus, inflammatory bowel diseases like ulcerative colitis and Crohn's disease, atopic dermatitis, ankylosing spondylitis, primary biliary cirrhosis, Guillain-Barre syndrome, chronic inflammatory demyelinating polyneuropathy, psoriasis, Grave's disease, Hashimoto's thyroiditis, Myasthenia gravis, vasculitis, celiac disease, Sjogren's syndrome, polymyalgia rheumatica, or any other condition where an autoimmune disease results from an elevation in circulating sIL7R.

In another embodiment, an antibody is administered to a patient. In a further embodiment, the antibody is administered to a patient by one or more different routes of administration. Routes of administration include, for example, intravenous, intraperitoneal, subcutaneously, intramuscular, oral, nasal, intravaginal, anally, intraocular and topically.

### Methods of Administration/Treatment

A pharmaceutical composition disclosed herein may optionally include a pharmaceutically-acceptable carrier that facilitates processing of an active ingredient into pharmaceutically-acceptable compositions. Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

A pharmaceutical composition or formulation disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition or formulation disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

In an embodiment, a pharmaceutical composition disclosed herein may further include a pharmaceutically-acceptable carrier, a pharmaceutically-acceptable component, or both pharmaceutically-acceptable carrier and pharmaceutically-acceptable component. In an embodiment, a therapeutic provides pharmacological activity or other direct effect in the cure, mitigation, treatment, or prevention of MS or autoimmune diseases and disorders, or to affect the structure or any function of the body of a human or an animal.

In one embodiment, a therapeutic, including an antibody disclosed herein is capable of reducing the severity of an autoimmune disorder in an afflicted subject by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic, including an antibody disclosed herein is capable of reducing the severity of an autoimmune disorder in an afflicted subject by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a therapeutic, including an antibody and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In another embodiment, the period of administration of a therapeutic, including an antibody is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In an embodiment, a first therapeutic, including an antibody is administered to an individual and at a later date, a second therapeutic, including an antibody is administered to the same individual. In an embodiment, a first therapeutic, including an antibody is administered to an individual at the same time as a second therapeutic, including an antibody is administered to the individual.

A therapeutic, including an antibody disclosed herein, or a pharmaceutical composition comprising a therapeutic, may be formulated for either local or systemic delivery using topical, enteral or parenteral routes of administration. Additionally, a therapeutic, including an antibody disclosed herein may be formulated by itself in a pharmaceutical composition, or may be formulated together with one or more other therapeutics disclosed herein in a single pharmaceutical composition.

A therapeutic, including antibodies disclosed herein, or a pharmaceutical composition comprising a therapeutic, may be made into an inhaled formulation. Inhaled formulations suitable for enteral or parenteral administration include, without limitation, aerosols, dry powders, solvents, gases and nitrites. A therapeutic, including an antibody or composition disclosed herein intended for such administration may be prepared according to any method known to the art for the manufacture of pharmaceutical compositions.

In such inhaled dosage forms, a therapeutic, including an antibody or a pharmaceutical composition may be prepared for delivery as an aerosol in a liquid propellant for use in a pressurised (PDI) or other metered dose inhaler (MDI). Propellants suitable for use in a PDI or MDI include, without limitation, CFC-12, HFA-134a, HFA-227, HCFC-22 (difluorochloromethane), HFA-152 (difluoroethane and isobutane). An MS therapeutic, including an antibody may also be delivered using a nebulisers or other aerosol delivery system. An MS therapeutic, including an antibody may be prepared for delivery as a dry powder for use in a dry powder inhaler (DPI). A dry powder for use in the inhalers will usually have a mass median aerodynamic diameter of less than 30 pm, preferably less than 20 pm and more preferably less than 10 pm. Microparticles having aerodynamic diameters in the range of about 5 pm to about 0.5 pm will generally be deposited in the respiratory bronchioles, whereas smaller particles, having aerodynamic diameters in the range of about 2 pm to about 0.05 pm, are likely to be deposited in the alveoli. A DPI may be a passive delivery mechanism, which relies on the individual's inspiration to introduce the particles into the lungs, or an active delivery mechanism, requiring a mechanism for delivering the powder to the individual. In inhalatory formulations, a therapeutically effective amount of a therapeutic disclosed herein for an inhaled formulation may be between about 0.0001% (w/v) to about 60% (w/v), about 0.001% (w/v) to about 40.0% (w/V), or about 0.01% (w/v) to about 20.0% (w/v). In inhalatory formulations, a therapeutically effective amount of a therapeutic disclosed herein for an inhaled formulation may also be between about 0.0001% (w/w) to about 60% (w/w), about 0.001% (w/w) to about 40.0% (w/w), or about 0.01% (w/w) to about 20.0% (w/w).

A therapeutic, including an antibody disclosed herein, or a pharmaceutical composition comprising a therapeutic, including an antibody, may be made into a solid formulation. Solid formulations suitable for enteral or parenteral administration include, without limitation, capsules, tablets, pills, troches, lozenges, powders and granules suitable for inhalation or for reconstitution into sterile injectable solutions or dispersions. A therapeutic, including an antibody or composition disclosed herein intended for such administration may be prepared according to any method known to the art for the manufacture of pharmaceutical compositions. In such solid dosage forms, the therapeutic, including an antibody may be admixed with (a) at least one inert customary excipient (or carrier), such as, e.g., sodium citrate or dicalcium phosphate or (b) fillers or extenders, as for example, starch, lactose, sucrose, glucose, mannitol, isomalt, and silicic acid, (c) binders, such as, e.g., carboxymethylcellulose, alignates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. In solid formulations, a therapeutically effective amount of a therapeutic, including an antibody disclosed herein typically may be between about 0.0001% (w/w) to about 60% (w/w), about 0.001% (w/w) to about 40.0% (w/w), or about 0.01% (w/w) to about 20.0% (w/w).

A therapeutic, including an antibody disclosed herein, or a pharmaceutical composition comprising a therapeutic, including an antibody, may be made into a semi-solid formulation. Semi-solid formulations suitable for topical administration include, without limitation, ointments, creams, salves, and gels. A therapeutic, including an antibody or composition disclosed herein intended for such administration may be prepared according to any method known to the art for the manufacture of pharmaceutical compositions. In semi-solid formulations, a therapeutically effective amount of a MS therapeutic disclosed herein typically may be between about 0.0001% (w/v) to about 60% (w/v), about 0.001% (w/v) to about 40.0% (w/v), or about 0.01% (w/v) to about 20.0% (w/v). In semi-solid formulations, a therapeutically effective amount of a therapeutic disclosed herein typically may also be between about 0.0001% (w/w) to about 60% (w/w), about 0.001% (w/w) to about 40.0% (w/w), or about 0.01% (w/w) to about 20.0% (w/w).

A therapeutic, including an antibody disclosed herein, or a pharmaceutical composition comprising a therapeutic, including an antibody, may be made into a liquid formulation. Liquid formulations suitable for enteral or parenteral administration include, without limitation, solutions, syrups, elixirs, dispersions, emulsions, and suspensions, including, but not limited, to those used for intravenous administration. A therapeutic, including an antibody therapeutic or composition disclosed herein intended for such administration may be prepared according to any method known to the art for the manufacture of pharmaceutical compositions. In such liquid dosage forms, a therapeutic, including an antibody or composition disclosed herein may be admixed with (a) suitable aqueous and nonaqueous carriers, (b) diluents, (c) solvents, such as, e.g., water, ethanol, propylene glycol, polyethyleneglycol, glycerol, vegetable oils, such as, e.g., rapeseed oil and olive oil, and injectable organic esters such as ethyl oleate; and/or fluidity agents, such as, e.g., surfactants or coating agents like lecithin. In the case of dispersions and suspensions, fluidity can also be controlled by maintaining a particular particle size. In liquid formulations, a therapeutically effective amount of a therapeutic, including an antibody disclosed herein typically may be between about 0.0001% (w/v) to about 60% (w/v), about 0.001% (w/v) to about 40.0% (w/v), or about 0.01% (w/v) to about 20.0% (w/v).

Liquid suspensions may be formulated by suspending a therapeutic, including an antibody disclosed herein in admixture with excipients suitable for the manufacture of aqueous suspensions. Such excipients are suspending agents, for example sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate.

Dispersible powders and granules suitable for preparation of an aqueous suspension by the addition of water may provide combined therapeutics in admixture with a dispersing or wetting agent, suspending agent and one or more preservatives.

A therapeutic, including an antibody disclosed herein, or a composition comprising a therapeutic, including an antibody, may also be incorporated into a therapeutic delivery platform in order to achieve a controlled release profile over time. A therapeutic, including an antibody delivery platform includes a MS therapeutic disclosed herein dispersed within a polymer matrix, typically a biodegradable, bioerodible, and/or bioresorbable polymer matrix.

In another embodiment, an MS therapeutic, including an antibody disclosed herein is capable of reducing the severity of MS in an individual suffering from MS by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an MS therapeutic, including an antibody disclosed herein is capable of reducing the severity of MS in an individual suffering from MS by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a MS therapeutic, including an antibody and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In another embodiment, the period of administration of an MS therapeutic, including an antibody is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In an embodiment, a first MS therapeutic, including an antibody is administered to an individual and at a later date, a second MS therapeutic, including an antibody is administered to the same individual. In an embodiment, a first MS therapeutic, including an antibody is administered to an individual at the same time as a second MS therapeutic, including an antibody is administered to the individual.

An MS therapeutic, including an antibody disclosed herein, or a pharmaceutical composition comprising such an MS therapeutic, may be formulated for either local or systemic delivery using topical, enteral or parenteral routes of administration. Additionally, an MS therapeutic, including an antibody disclosed herein may be formulated by itself in a pharmaceutical composition, or may be formulated together with one or more other MS therapeutics disclosed herein in a single pharmaceutical composition.

An MS therapeutic, including MS antibody disclosed herein, or a pharmaceutical composition comprising such a MS therapeutic, may be made into an inhaled formulation. Inhaled formulations suitable for enteral or parenteral administration include, without limitation, aerosols, dry powders, solvents, gases and nitrites. An MS therapeutic, including an antibody or composition disclosed herein intended for such administration may be prepared according to any method known to the art for the manufacture of pharmaceutical compositions.

In such inhaled dosage forms, an MS therapeutic, including an antibody or a pharmaceutical composition may be prepared for delivery as an aerosol in a liquid propellant for use in a pressurised (PDI) or other metered dose inhaler (MDI). Propellants suitable for use in a PDI or MDI include, without limitation, CFC-12, HFA-134a, HFA-227, HCFC-22 (difluorochloromethane), HFA-152 (difluoroethane and isobutane). An MS therapeutic, including an antibody may also be delivered using a nebulisers or other aerosol delivery system. An MS therapeutic, including an antibody may be prepared for delivery as a dry powder for use in a dry powder inhaler (DPI). A dry powder for use in the inhalers will usually have a mass median aerodynamic diameter of less than 30 pm, preferably less than 20 pm and more preferably less than 10 pm. Microparticles having aerodynamic diameters in the range of about 5 pm to about 0.5 pm will generally be deposited in the respiratory bronchioles, whereas smaller particles, having aerodynamic diameters in the range of about 2 pm to about 0.05 pm, are likely to be deposited in the alveoli. A DPI may be a passive delivery mechanism, which relies on the individual's inspiration to introduce the particles into the lungs, or an active delivery mechanism, requiring a mechanism for delivering the powder to the individual. In inhalatory formulations, a therapeutically effective amount of a MS therapeutic disclosed herein for an inhaled formulation may be between about 0.0001% (w/v) to about 60% (w/v), about 0.001% (w/V) to about 40.0% (w/v), or about 0.01% (w/v) to about 20.0% (w/v). In inhalatory formulations, a therapeutically effective amount of a MS therapeutic disclosed herein for an inhaled formulation may also be between about 0.0001% (w/w) to about 60% (w/w), about 0.001% (w/w) to about 40.0% (w/w), or about 0.01% (w/w) to about 20.0% (w/w).

An MS therapeutic, including an antibody disclosed herein, or a pharmaceutical composition comprising such an MS therapeutic, including an antibody, may be made into a solid formulation. Solid formulations suitable for enteral or parenteral administration include, without limitation, capsules, tablets, pills, troches, lozenges, powders and granules suitable for inhalation or for reconstitution into sterile injectable solutions or dispersions. An MS therapeutic, including an antibody or composition disclosed herein intended for such administration may be prepared according to any method known to the art for the manufacture of pharmaceutical compositions. In such solid dosage forms, the MS therapeutic, including an antibody may be admixed with (a) at least one inert customary excipient (or carrier), such as, e.g., sodium citrate or dicalcium phosphate or (b) fillers or extenders, as for example, starch, lactose, sucrose, glucose, mannitol, isomalt, and silicic acid, (c) binders, such as, e.g., carboxymethylcellulose, alignates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. In solid formulations, a therapeutically effective amount of an MS therapeutic, including an antibody disclosed herein typically may be between about 0.0001% (w/w) to about 60% (w/w), about 0.001% (w/w) to about 40.0% (w/w), or about 0.01% (w/w) to about 20.0% (w/w).

An MS therapeutic, including an antibody disclosed herein, or a pharmaceutical composition comprising such a MS therapeutic, including an antibody, may be made into a semi-solid formulation. Semi-solid formulations suitable for topical administration include, without limitation, ointments, creams, salves, and gels. An MS therapeutic, including an antibody or composition disclosed herein intended for such administration may be prepared according to any method known to the art for the manufacture of pharmaceutical compositions. In semi-solid formulations, a therapeutically effective amount of a MS therapeutic disclosed herein typically may be between about 0.0001% (w/v) to about 60% (w/v), about 0.001% (w/V) to about 40.0% (w/v), or about 0.01% (w/v) to about 20.0% (w/v). In semi-solid formulations, a therapeutically effective amount of a MS therapeutic disclosed herein typically may also be between about 0.0001% (w/w) to about 60% (w/w), about 0.001% (w/w) to about 40.0% (w/w), or about 0.01% (w/w) to about 20.0% (w/w).

An MS therapeutic, including an antibody disclosed herein, or a pharmaceutical composition comprising such an MS therapeutic, including an antibody, may be made into a liquid formulation. Liquid formulations suitable for enteral or parenteral administration include, without limitation, solutions, syrups, elixirs, dispersions, emulsions, and suspensions, including, but not limited, to those used for intravenous administration. An MS therapeutic, including an antibody therapeutic or composition disclosed herein intended for such administration may be prepared according to any method known to the art for the manufacture of pharmaceutical compositions. In such liquid dosage forms, an MS therapeutic, including an antibody or composition disclosed herein may be admixed with (a) suitable aqueous and nonaqueous carriers, (b) diluents, (c) solvents, such as, e.g., water, ethanol, propylene glycol, polyethyleneglycol, glycerol, vegetable oils, such as, e.g., rapeseed oil and olive oil, and injectable organic esters such as ethyl oleate; and/or fluidity agents, such as, e.g., surfactants or coating agents like lecithin. In the case of dispersions and suspensions, fluidity can also be controlled by maintaining a particular particle size. In liquid formulations, a therapeutically effective amount of an MS therapeutic, including an antibody disclosed herein typically may be between about 0.0001% (w/v) to about 60% (w/v), about 0.001% (w/v) to about 40.0% (w/v), or about 0.01% (w/v) to about 20.0% (w/v).

Syrups and elixirs may be formulated with sweetening agents, for example glycerol, propylene glycol, sorbitol or sucrose. Such formulations may also contain a demulcent, a preservative, flavoring agents, and coloring agents.

Liquid suspensions may be formulated by suspending an MS therapeutic, including an antibody disclosed herein in admixture with excipients suitable for the manufacture of aqueous suspensions. Such excipients are suspending agents, for example sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate.

Dispersible powders and granules suitable for preparation of an aqueous suspension by the addition of water provide the combined MS therapeutics in admixture with a dispersing or wetting agent, suspending agent and one or more preservatives.

An MS therapeutic, including an antibody disclosed herein, or a composition comprising such an MS therapeutic, including an antibody, may also be incorporated into an MS therapeutic delivery platform in order to achieve a controlled release profile over time. Such an MS therapeutic, including an antibody delivery platform includes a MS therapeutic disclosed herein dispersed within a polymer matrix, typically a biodegradable, bioerodible, and/or bioresorbable polymer matrix.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the MS therapeutic delivery platform. Examples of biodegradable, bioerodible, and/or bioresorbable polymers and methods useful to make a MS therapeutic delivery platform are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hyrophilic and Hydrophobic Agents, U.S. Pat. No. 7,048,946; Hughes, et. al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. Patent Publication 2011/0008437; each of which is incorporated by reference in its entirety.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

One of ordinary skill in the art will appreciate that the selection of a suitable polymer for forming a suitable disclosed MS or autoimmune therapeutic delivery platform depends on several factors. The more relevant factors in the selection of the appropriate polymer(s), include, without limitation, compatibility of polymer with a MS therapeutic, desired release kinetics of an MS therapeutic, including an antibody, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, dictate the in vitro and in vivo behavior of the polymer include the chemical composition, spatial distribution of the constituents, the molecular weight of the polymer and the degree of crystallinity.

An MS therapeutic, including an antibody delivery platform includes both a sustained release MS therapeutic delivery platform and an extended release MS therapeutic delivery platform.

In aspects of this embodiment, a sustained release MS therapeutic delivery platform releases an MS therapeutic, including an antibody disclosed herein with substantially zero order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release MS therapeutic delivery platform releases an MS therapeutic, including an antibody disclosed herein with substantially zero order release kinetics over a period of, e.g., at least 7 days after administration, at least 8 days after administration, at least 9 days after administration, at least 10 days after administration, at least 12 days after administration, at least 15 days after administration, at least 20 days after administration, at least 25 days after administration, at least 30 days after administration, at least 45 days after administration, at least 60 days after administration, at least 75 days after administration, or at least 90 days after administration or more.

In aspects of this embodiment, a sustained release MS therapeutic, including an antibody delivery platform releases a therapeutic disclosed herein with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release MS therapeutic delivery platform releases an MS therapeutic, including an antibody disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, an MS therapeutic, including an antibody delivery platform releases an MS therapeutic, including an antibody disclosed herein with substantially zero order release kinetics over a period of, e.g., about 1 day after administration, about 2 days after administration, about 3 days after administration, about 4 days after administration, about 5 days after administration, or about 6 days after administration. In other aspects of this embodiment, an MS therapeutic, including an antibody delivery platform releases a MS therapeutic disclosed herein with substantially zero order release kinetics over a period of, e.g., at most 1 day after administration, at most 2 days after administration, at most 3 days after administration, at most 4 days after administration, at most 5 days after administration, or at most 6 days after administration.

In aspects of this embodiment, an MS therapeutic, including an antibody delivery platform releases an MS therapeutic, including an antibody disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, an MS therapeutic, including an antibody delivery platform releases a MS therapeutic disclosed herein with substantially first order release kinetics over a period of, e.g., at most 1 day after administration, at most 2 days after administration, at most 3 days after administration, at most 4 days after administration, at most 5 days after administration, or at most 6 days after administration.

In aspects of this embodiment, the amount of an MS therapeutic, including an antibody that is contacted with the solvent in step (a) may be, e.g., at least 10 mg, at least 20 mg, at least 30 mg, at least 40 mg, at least 50 mg, at least 60 mg, at least 70 mg, at least 80 mg, at least 90 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg. In other aspects of this embodiment, the amount of an MS therapeutic, including an antibody that is contacted with the solvent in step (a) may be in the range of, e.g., about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg. In other aspects of this embodiment, the amount of an MS therapeutic, including an antibody that is dissolved in the solvent in step (a) may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, or about 200 mg to about 1,500 mg.

In an embodiment, an MS therapeutic, including an antibody disclosed herein may be in any concentration desired. In aspects of this embodiment, the concentration of an MS therapeutic, including an antibody disclosed herein may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of an MS therapeutic, including an antibody disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL. In other aspects of this embodiment, the concentration of an MS therapeutic, including an antibody disclosed herein on may be in a range of, e.g., about 0.00001 mg/mL to about 3,000 mg/mL, about 0.0001 mg/mL to about 3,000 mg/mL, about 0.001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/ml to about 100 mg/mL.

The final concentration of an MS therapeutic, including an antibody disclosed herein in a pharmaceutical composition disclosed herein may be of any concentration desired. In an aspect of this embodiment, the final concentration of an MS therapeutic, including an antibody in a pharmaceutical composition may be a therapeutically effective amount. In other aspects of this embodiment, the final concentration of an MS therapeutic, including an antibody in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of an MS therapeutic, including an antibody disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL. In other aspects of this embodiment, the final concentration of an MS therapeutic, including an antibody in a pharmaceutical composition may be in a range of, e.g., about 0.00001 mg/mL to about 3,000 mg/mL, about 0.0001 mg/ml to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

In an embodiment, an MS therapeutic, including an antibody reduces the severity of a symptom associated with an autoimmune disease by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% as compared to a patient not receiving the same treatment.

In an embodiment, an MS therapeutic, including an antibody reduces the severity of a symptom associated with an autoimmune disease by of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% as compared to a patient not receiving the same treatment.

In an embodiment, an MS therapeutic, including an antibody reduces the severity of a symptom associated with an autoimmune disease no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% as compared to a patient not receiving the same treatment.

In an embodiment, an antibody that neutralizes sIL7R reduces the severity of a symptom associated with an autoimmune disease by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% as compared to a patient not receiving the same treatment.

In an embodiment, an antibody that neutralizes sIL7R reduces the severity of a symptom associated with an autoimmune disease by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% as compared to a patient not receiving the same treatment.

In an embodiment, an antibody that neutralizes sIL7R reduces the severity of a symptom associated with an autoimmune disease by no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% as compared to a patient not receiving the same treatment.

In another embodiment, an antibody that neutralizes sIL7R reduces the severity of a symptom associated with MS by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% as compared to a patient not receiving the same treatment.

In another embodiment, an antibody that neutralizes sIL7R reduces the severity of a symptom associated with MS, by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% as compared to a patient not receiving the same treatment.

In another embodiment, an antibody that neutralizes sIL7R reduces the severity of a symptom associated with MS, by no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% as compared to a patient not receiving the same treatment.

In an embodiment of the present invention, an antibody that neutralizes sIL7R, or reduces its levels, has been modified to have a homology of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% as compared to the natural, unmodified antibody from which the antibody that has been modified was derived.

In an embodiment of the present invention, an antibody that neutralizes sIL7R, or reduces its levels, has been modified to have a homology of by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% as compared to the natural, unmodified antibody from which the antibody that has been modified was derived.

In an embodiment of the present invention, an antibody that neutralizes sIL7R, or reduces its levels, has been modified to have a homology of no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% as compared to the natural, unmodified antibody from which the antibody that has been modified was derived.

In an embodiment of the present invention, an MS therapeutic, including an antibody that neutralizes sIL7R (or reduces its levels), increases the cognitive function, decreases vision problems, decreases fatigue, decreases heat sensitivity, reduces bowel and bladder problems, increases sexual function, decreases pain and/or motor problems suffered by a patient by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% as compared to a patient that does not receive the MS therapeutic.

In an embodiment of the present invention, an MS therapeutic, including an antibody that neutralizes sIL7R, or reduces its levels, increases cognitive function, decreases vision problems, decreases fatigue, decreases heat sensitivity, reduces bowel and bladder problems, increases sexual function, decreases pain and/or motor problems suffered by a patient by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% as compared to a patient that does not receive the MS therapeutic.

In an embodiment of the present invention, an MS therapeutic, including an antibody that neutralizes sIL7R, or reduces its levels, increases cognitive function, decreases vision problems, decreases fatigue, decreases heat sensitivity, reduces bowel and bladder problems, increases sexual function, decreases pain and/or motor problems suffered by a patient by no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% as compared to a patient that does not receive the MS therapeutic.

In an embodiment of the present invention, a therapeutic, including an antibody that neutralizes sIL7R, or reduces its levels, increases the cognitive function, decreases vision problems, decreases fatigue, decreases heat sensitivity, reduces bowel and bladder problems, increases sexual function, decreases pain and/or motor problems suffered by a patient by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% as compared to a patient that does not receive the therapeutic.

In an embodiment of the present invention, a therapeutic, including an antibody that neutralizes sIL7R, or reduces its levels, increases cognitive function, decreases vision problems, decreases fatigue, decreases heat sensitivity, reduces bowel and bladder problems, increases sexual function, decreases pain and/or motor problems suffered by a patient by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% as compared to a patient that does not receive the therapeutic.

In an embodiment of the present invention, a therapeutic, including an antibody that neutralizes sIL7R, or reduces its levels, increases cognitive function, decreases vision problems, decreases fatigue, decreases heat sensitivity, reduces bowel and bladder problems, increases sexual function, decreases pain and/or motor problems suffered by a patient by no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% as compared to a patient that does not receive the therapeutic.

In another embodiment, the severity of an autoimmune disease in a patient caused by circulating sIL7R is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% as compared to a patient that does not receive an antibody that neutralizes sIL7R.

In another embodiment, the severity of an autoimmune disease in a patient caused by circulating sIL7R is reduced by no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% as compared to a patient that does not receive an antibody that neutralizes sIL7R.

In yet another embodiment, the severity of an autoimmune disease in a patient caused by circulating sIL7R is reduced by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% as compared to a patient that does not receive an antibody that neutralizes sIL7R.

In another embodiment, the antibody that neutralizes sIL7R (or reduces its levels) comprises at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% of a pharmaceutical composition.

In another embodiment, the antibody that neutralizes sIL7R (or reduces its levels) comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% of a pharmaceutical composition.

In another embodiment, the antibody that neutralizes sIL7R (or reduces its levels) comprises no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, no more than 50%, no more than 51%, no more than 52%, no more than 53%, no more than 54%, no more than 55%, no more than 56%, no more than 57%, no more than 58%, no more than 59%, no more than 60%, no more than 61%, no more than 62%, no more than 63%, no more than 64%, no more than 65%, no more than 66%, no more than 67%, no more than 68%, no more than 69%, no more than 70%, no more than 71%, no more than 72%, no more than 73%, no more than 74%, no more than 75%, no more than 76%, no more than 77%, no more than 78%, no more than 79%, no more than 80%, no more than 81%, no more than 82%, no more than 83%, no more than 84%, no more than 85%, no more than 86%, no more than 87%, no more than 88%, no more than 89%, no more than 90%, no more than 91%, no more than 92%, no more than 93%, no more than 94%, no more than 95%, no more than 96%, no more than 97%, no more than 98%, no more than 99% or no more than 100% of a pharmaceutical composition.

The monoclonal antibodies generated here are specific to sIL7R and do not bind the membrane-bound IL7R (mIL7R) in the surface of T cells and monocytes to avoid immunosuppression. To generate sIL7R-specific monoclonal antibodies we targeted regions that are specific for sIL7R. Since sIL7R is produced by exclusion of the sixth exon from IL7R pre-mRNAs, the new junction between exon 5 and exon 7 creates a unique 26 amino acid C-terminal sequence for sIL7R. We targeted the sequence encoded at the junction between exon 5 and exon 7, as well as the unique C-terminal tail sequence as described below.

### EXAMPLES

The following non-limiting examples are provided for illustrative purposes only in order to facilitate a more complete understanding of representative embodiments now contemplated. These examples are intended to be a mere subset of all possible contexts in which the components of the formulation or composition may be combined. Thus, these examples should not be construed to limit any of the embodiments described in the present specification, including those pertaining to the type and amounts of components of the formulation and/or methods and uses thereof.

### Example 1

Amino Acid Sequence of sIL7R C-Terminal Tail Targeted with Antibodies:

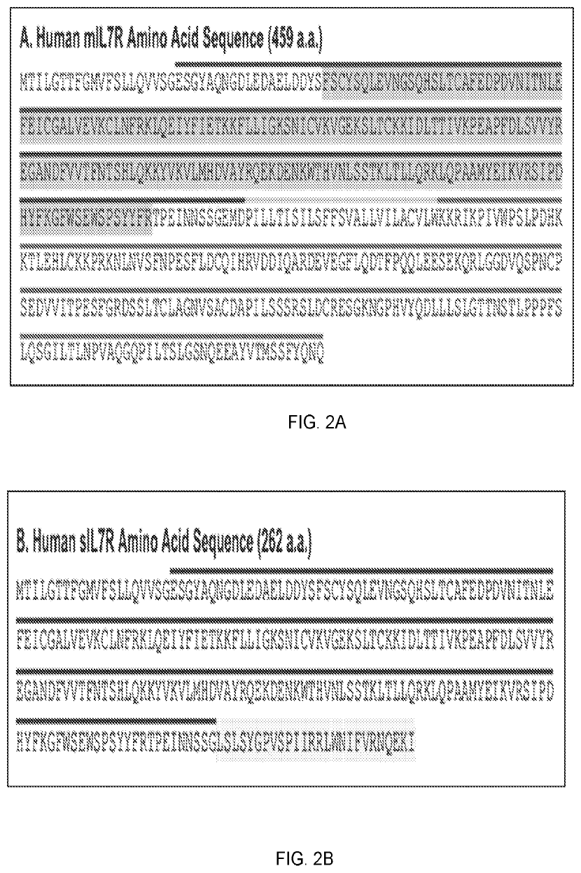

In this example, antibodies were produced to specific regions of sIL7R (i.e., regions that are not shared with mIL7R). The amino acid sequences of human mIL7R and sIL7R isoforms are compared in FIGS. 2A and 2B. Sequences corresponding to the different domains are distinguished by color-coded lines above the sequence.

FIG. 2A shows the amino acid sequence for human mIL7R with the sequence corresponding to the different domains of sIL7R.

Domains in mIL7R are as Follows:
extracellular domain (red line; 219 residues): ESGYAQ . . . SGEMD
transmembrane domain (orange line; 25 residues): PILLT . . . ACVLW
intracellular domain (blue line; 195 residues): KKRIK . . . FYQNQ

The sequence highlighted in cyan contains the residues that interact with IL7.

FIG. 2B shows that sIL7R includes an extracellular domain (red line; 219 residues) with a C-terminal tail (yellow highlight; 26 residues). Exclusion of exon 6 from IL7R pre-mRNAs changes the reading frame and creates a 26 amino acid C-terminal tail prior to the occurrence of a premature termination codon (PTC) in the last exon. Because the PTC is located in the last exon, transcripts that exclude exon 6 are not substrates to nonsense-mediated RNA decay and are effectively translated into sIL7R protein.

FIG. 2B shows the amino acid sequence for human sIL7R with the sequence corresponding to the different domains of mIL7R.

Domains in sIL7R are as Follows:
extracellular domain (red line; 219 residues): ESGYA . . . NNSSG
a C-terminal tail (yellow highlight; 26 residues): LSLSY . . . NQEKI

Exclusion of exon 6 from IL7R pre-mRNAs changes the reading frame and creates a 26 amino acid C-terminal tail prior to the occurrence of a premature termination codon (PTC) in the last exon. Because the PTC is located in the last exon, transcripts that exclude exon 6 are not substrates to nonsense-mediated RNA decay and are effectively translated into sIL7R protein.

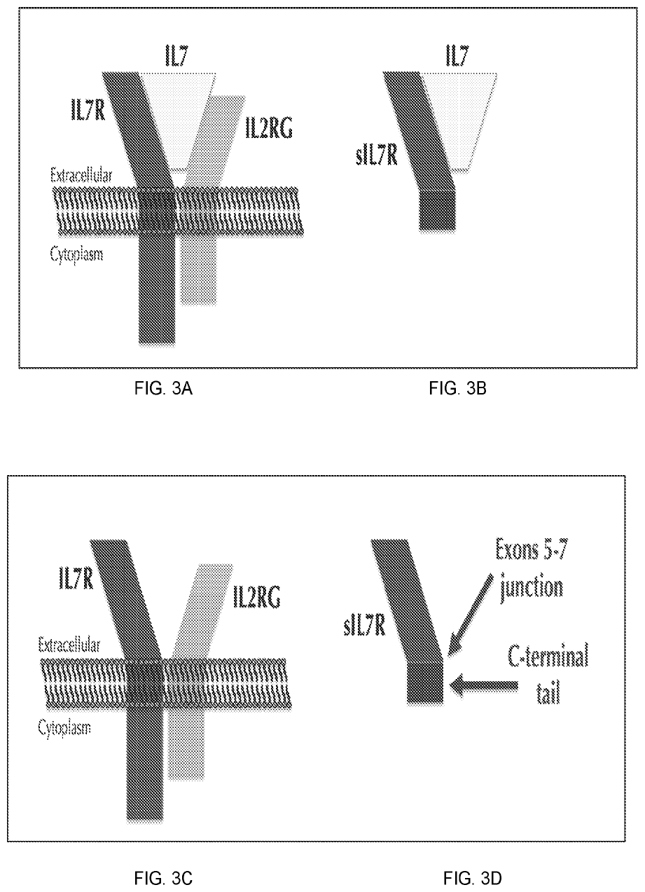

FIGS. 3A, 3B, 3C and 3D depict the arrangement of the membrane-bound receptor complex for IL7 composed of mIL7R and the common cytokine receptor gamma chain (IL2RG) with and without IL7. FIGS. 3A and 3C illustrate the arrangement of the membrane-bound receptor complex for IL7 composed of mIL7R and the common cytokine receptor gamma chain (IL2RG) with (3A) and without (3C) IL7. mIL7R is shown in blue, IL2RG is shown in green, and IL7 is shown in yellow. FIGS. 3B and 3D illustrate the structure of sIL7R with (3B) and without (3D) IL7. sIL7R is shown in blue with the C-terminal tail in red, and IL7 in yellow.

In FIG. 3D, arrows indicate the two regions of sIL7R targeted by the anti-sIL7R antibodies: (1) the sIL7R-specific C-terminal tail (encoded by exon 7 in transcripts that skip exon 6), and (2) the region of sIL7R encoded by the junction between exons 5 and 7 in transcripts that skip exon 6. Both regions are specific to sIL7R and are predicted to prevent binding of IL7 to sIL7R but not to mIL7R.

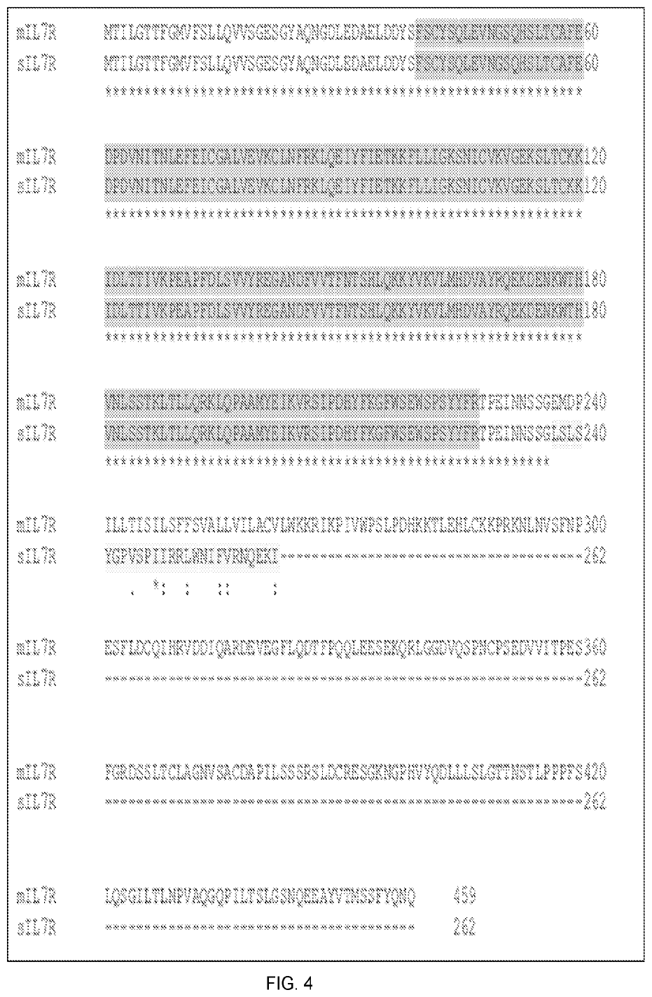

Similarly, FIG. 4 shows amino acid sequence alignment of human mIL7R and sIL7R illustrating the sIL7R-specific C-terminal tail. The sequence of the extracellular domain involved in binding IL7 is highlighted in cyan (FSCYS . . . SYYFR), whereas the C-terminal tail specific for sIL7R is highlighted in yellow (LSLSY . . . NQEKI).

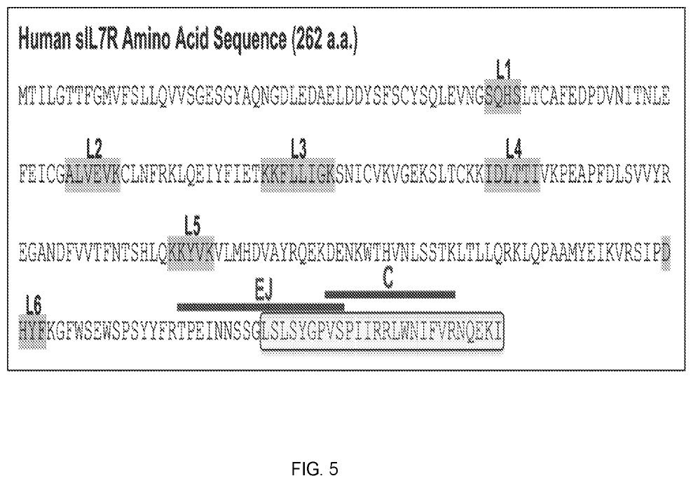

FIG. 5 shows the amino acid sequence of human sIL7R illustrating the IL7 binding region, the sIL7R-specific C-terminal tail and the junction between them. Sequences within the extracellular domain presumed to interact with IL7 are highlighted in green (L1-L6), whereas the C-terminal tail specific for sIL7R is highlighted with a yellow box.

Two antigens were selected for development of anti-sIL7R monoclonal antibodies that discriminate within sIL7R and mIL7R: one of the targeted antigens is located within the sIL7R-specific C-terminal tail (C, red line), whereas the other spans the junction between the extracellular domain and the C-terminal tail (EJ, blue line).

Standard monoclonal antibody techniques known in the art were used to produce an antibody to bind to the C-terminal tail of a sIL7R. The monoclonal antibody contains complementary determining regions (“CDR”) or (“CDRs”) that are capable of binding to at least 2, 3, 4, 5, 6, 7, 8, 9, 10, 11, 12, 13, 14, 15, 16, 17, 18, 19, 20, 21, 22, 23, 24, 25 or 26 amino acids of the C-terminal tail of sIL7R, wherein the amino acid sequence anywhere in the C-terminal tail is: LSLSYGPVSPIIRRLWNIFVRNQEKI. (SEQ ID No. 1).

### Example 2

Amino Acid Sequence of sIL7R Region Involved in Binding IL7

In this example, standard monoclonal antibody techniques were used to produce an antibody to bind to regions of sIL7R involved in IL7 binding. The monoclonal antibody contains complementary determining regions (“CDR”) or (“CDRs”) that are capable of binding to at least 2, 3, 4, 5, 6, 7, 8, 9, 10, 11, 12, 13, 14, 15, 16, 17, 18, 19, 20, 21, 22, 23, 24, 25, 26 or more amino acids of the region of sIL7R involved in binding IL7, wherein the amino acid sequence of the region involved in binding IL7 is:

(SEQ ID No. 2)
FSCYSQLEVNGSQHSLTCAFEDPDVNITNLEFEICGALVEVKCLNFRKL
QEIYFIETKKFLLIGKSNICVKVGEKSLTCKKIDLTTIVKPEAPFDLSV
VYREGANDFVVTFNTSHLQKKYVKVLMHDVAYRQEKDENKWTHVNLSST
KLTLLQRKLQPAAMYEIKVRSIPDHYFKGFWSEWSPSYYFR.

### Example 3

Amino Acid Sequence of sIL7R at the Junction Between the IL7 Binding Region and the C-Terminal Tail Region

A sIL7R peptide was produced by exclusion of exon 6 from IL7R pre-mRNAs, where exon 5 is now joined to exon 7 instead of exon 6, thereby creating a new sequence specific for sIL7R (C-terminal tail). The two regions of sIL7R that we targeted are those that are specific for SIL7R and not present in mIL7R (see below).

Standard monoclonal antibody techniques were used to produce an antibody to bind to regions of sIL7R at the junction between the IL7 binding region and the C-terminal tail region. The monoclonal antibody contains complementary determining regions (“CDR”) or (“CDRs”) that are capable of binding to at least 2, 3, 4, 5, 6, 7, 8, 9, 10, 11, 12, 13, 14, 15, 16, 17, 18, 19, 20, 21, 22, 23, 24, 25 or 26 amino acids of the region of sIL7R at the junction between the region involved in binding IL7 and the C-terminal tail, wherein the amino acid sequence of the region involved in binding IL7 is: PDHYFKGFWSEWSPSYYFRTPEINNSSGLSLSYGPVSPIIRRLWNIFVRNQEKI (SEQ ID No. 3).

### Example 4

Amino Acid Sequence of sIL7R at the Junction Between Exon 5 and Exon 7

Standard monoclonal antibody techniques known in the art were used to generate antibodies to bind to regions of sIL7R at the junction between exon 5 and exon 7, where the junction between exon 5 and exon 7 occurs after NNSSG in SEQ ID No. 3 (see above, bold/underlined portion). This peptide of 18 amino acids was used to generate the mAbs. The monoclonal antibody contains complementary determining regions (“CDR”) or (“CDRs”) that are capable of binding to at least 2, 3, 4, 5, 6, 7, 8, 9, 10, 11, 12, 13, 14, 15, 16, 17, 18, 19, 20, 21, 22, 23, 24, 25, 26 more amino acids anywhere in the region of sIL7R at the junction between exon 5 and exon 7.

### Example 5

### ELISA Screening of mAB Clones

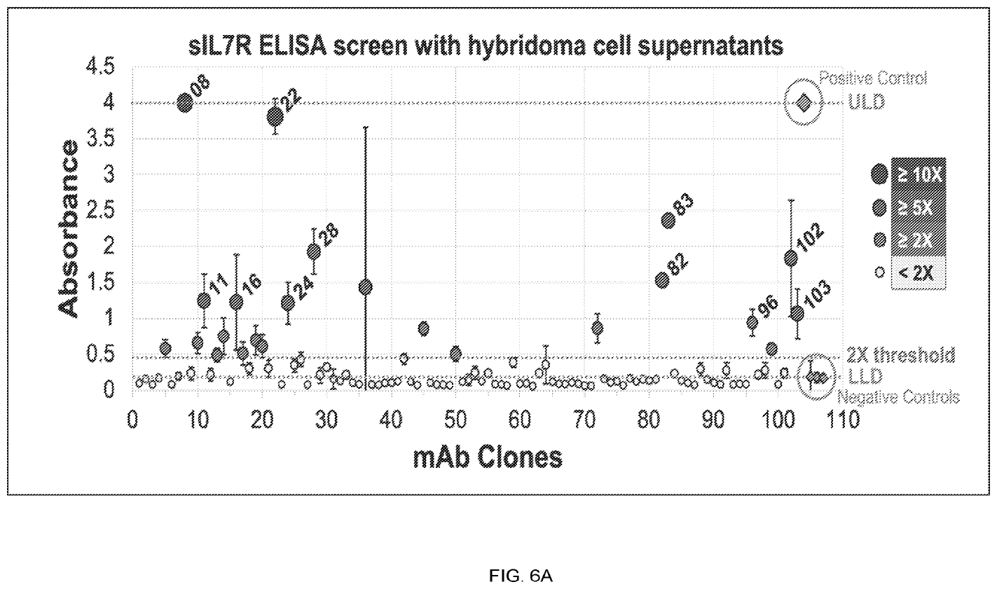

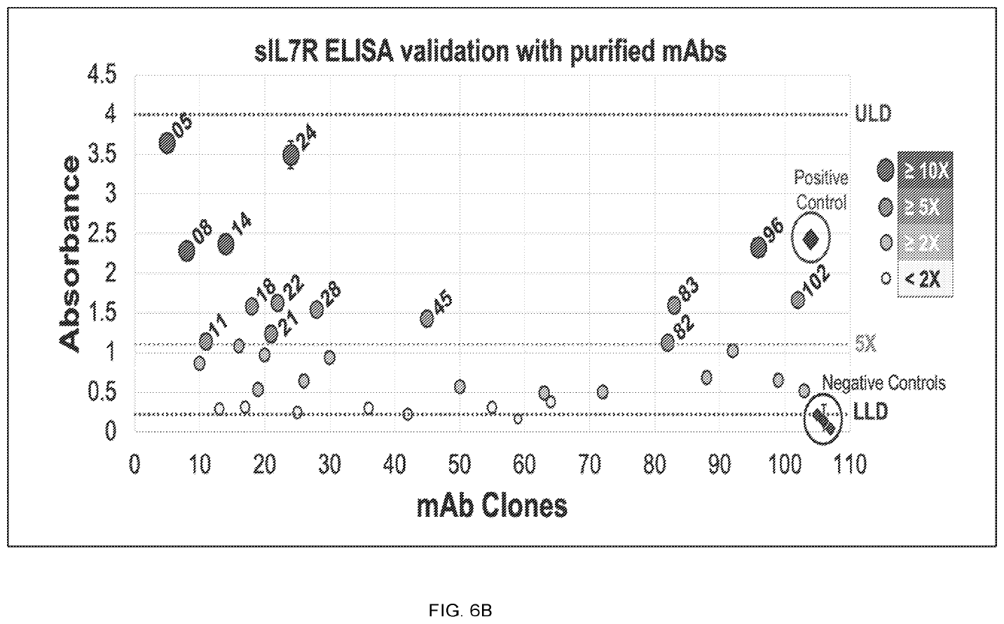

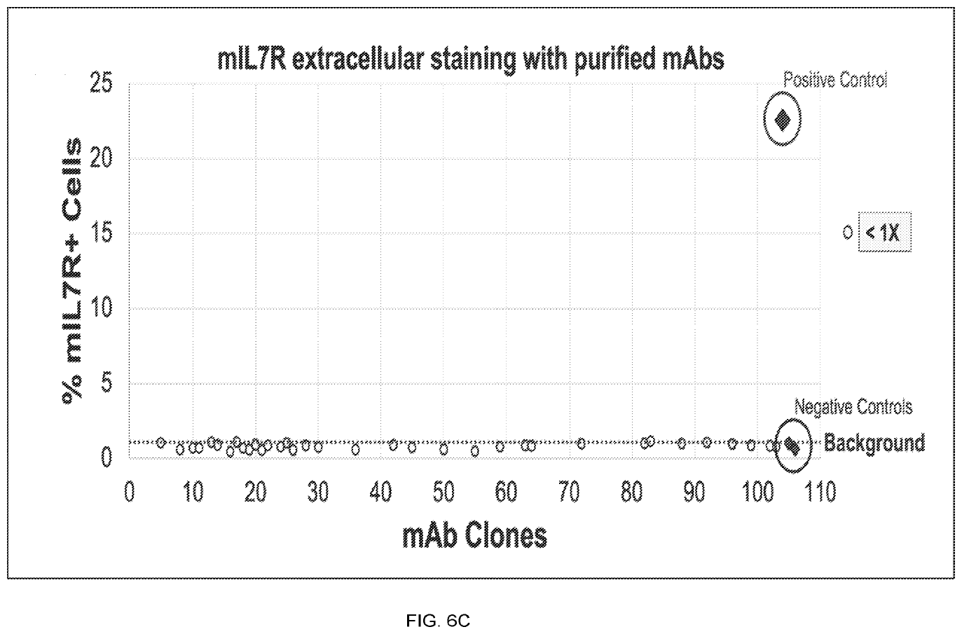

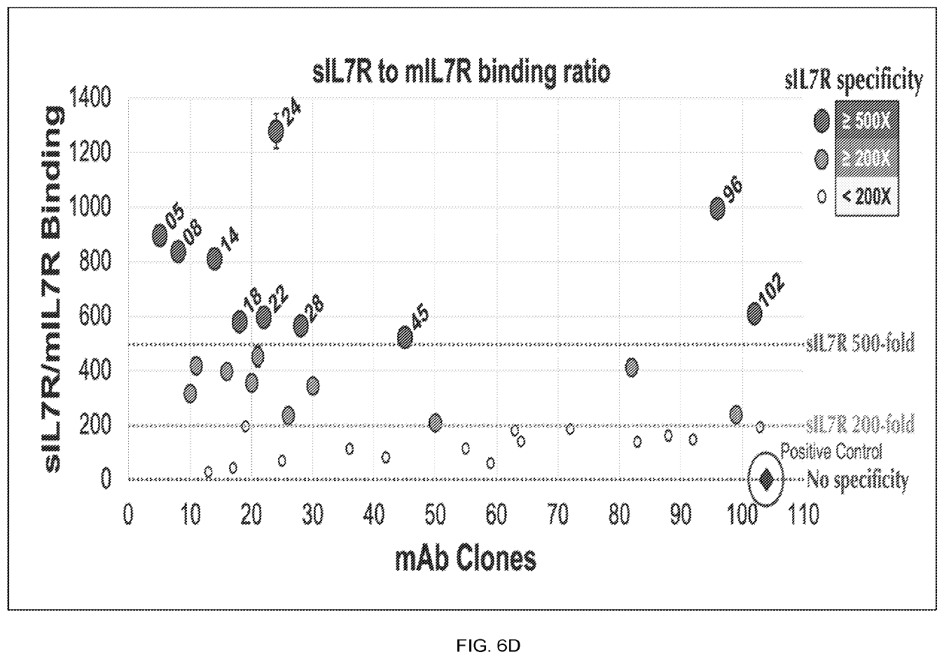

FIG. 6A shows the results of screening anti-sIL7R mAb clones binding to sIL7R by ELISA assay using hybridoma cell supernatants. Supernatants (100 μl) from 103 hybridoma cells were screened for binding to sIL7R in ELISA assays. Blue and red dotted lines indicate the lower (LLD) and upper (ULD) limit of detection of the assay, respectively. A validated anti-IL7R mAb (clone 40131; red diamond) was used as positive control. 35 clones that scored above background were selected for further analyses using purified mAbs to identify sIL7R-specific clones.

FIG. 6B shows the results of screening anti-sIL7R mAb clones binding to sIL7R by ELISA assay using purified antibodies. Purified mAbs from the 35 positive clones were used as detection antibody at 10 μg/ml in ELISA assays. Blue and red dotted lines indicate the lower (LLD) and upper (ULD) limit of detection of the assay, respectively. A validated anti-IL7R mAb (clone 40131; red diamond) was used as positive control. 27 clones bound to sIL7R at an arbitrary cutoff of two times over background, 14 of which bound to sIL7R at an arbitrary cutoff of five times over background.

FIG. 6C shows the results of an assessment of anti-sIL7R mAb clones binding to mIL7R by extracellular staining of human primary CD4⁺ T cells using purified antibodies. Purified mAbs (10 μg/ml) of the 35 positive clones were tested for binding to mIL7R by extracellular staining of intact human primary T cells and mean fluorescence intensity (MFI) was quantified by flow cytometry. The blue dotted line indicates the lower limit of detection (LLD) of the assay. A validated anti-IL7R mAb (clone 40131; red diamond) was used as positive control. Whereas the positive control bound to mIL7R in the surface of the stained T cells, none of the 35 mAb clones did.

FIG. 6D shows sIL7R specificity (sIL7R/mIL7R binding ratio). Signals for sIL7R (FIG. 6B) and mIL7R (FIG. 6C) were normalized to the positive control (clone 40131; red diamond) and used to calculate the sIL7R/mIL7R binding ratio, wherein sIL7R/mIL7R>1 equals specificity for SIL7R. Red dotted line indicates no specificity for sIL7R (sIL7R/mIL7R=1), whereas green dotted lines indicate the sIL7R-specificity thresholds of 200-fold (sIL7R/mIL7R>200) and 500-fold (sIL7R/mIL7R>500).

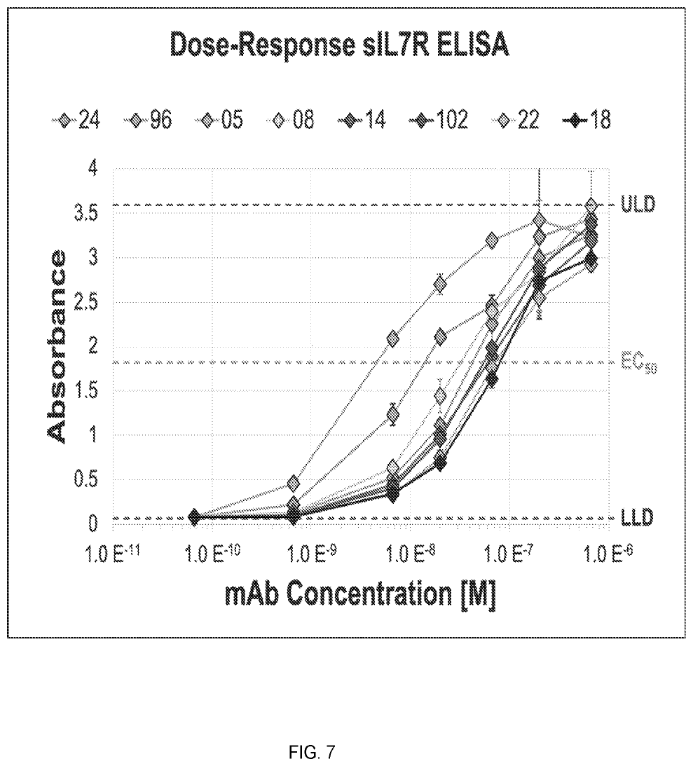

FIG. 7 shows sIL7R dose-response binding curves of top α-sIL7R mAb clones. Dose-response curves of the top eight clones (05, 08, 14, 18, 22, 24, 96 & 102) binding to sIL7R were determined by ELISA using purified mAbs as detection antibody at the indicated concentrations. Blue and red dashed lines indicate the respective lower (LLD) and upper (ULD) limit of detection, whereas the green dashed line indicates apparent EC₅₀. All the antibodies bound to sIL7R with high affinity (low nM range) and their apparent EC₅₀ values were as follow (in ascending order): 05 (4 nM), 24 (13 nM), 08 (30 nM), 96 (40 nM), 102 (53 nM) 14 (60 nM), 22 (71 nM) and 18 (80 nM). Table 1 (below) is a summary of the mAb clones and the sequences of heavy and light chains.

### Example 6

Inhibition of sIL7R Activity

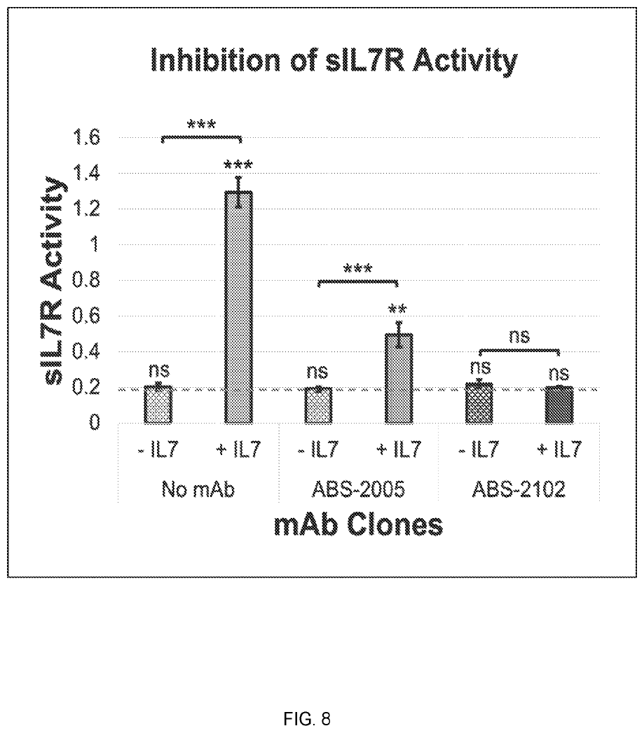

FIG. 8 shows inhibition of sIL7R activity by lead α-sIL7R mAb clones. The two clones with the lowest EC₅₀, clones 05 (ABS-2005) and 102 (ABS-2102), were tested for their ability to inhibit sIL7R. Inhibition of sIL7R was assessed by quantification of the IL7-induced gene BCL2, whose expression is enhanced by sIL7R. Human primary CD4+ T cells were cultured for two days to let secreted sIL7R accumulate in the media. The cells were then incubated with α-sIL7R mAbs for three hours, followed by treatment with 1 ng/ml of human recombinant IL7 for 24 hours, and quantification of BCL2 by qRT-PCR. Statistical significance was assessed by student's t test against the baseline expression of BCL2 (dashed line) or as indicated by the lines. BCL2 expression was induced by IL7 treatment in the absence of the mAb clones (active sIL7R), but not in the presence of anti-sIL7R clones 05 (ABS-2005) or 102 (ABS-2102), indicating inhibition of sIL7R activity.

### Example 7

### Administration of Monoclonal Antibody to Treat Multiple Sclerosis

In Multiple Sclerosis (MS), the immune system attacks the protective sheath that covers nerve fibers and causes communication problems between the brain and the rest of the body. Eventually, the disease can cause permanent damage or deterioration of the nerve fibers. Signs and symptoms of MS vary widely between patients and depend on the location and severity of nerve fiber damage in the central nervous system.

In this example, a 34-year-old female visits a healthcare professional and complains of several issues:
Numbness/weakness of her more limbs on one side of your body,
Tingling sensations
Lack of coordination
Unsteady gait and occasional inability to walk
Blurry vision
Vertigo

The practitioner conducts a thorough examination and diagnoses the patient with MS. The patient is treated with intravenous administration of a sterile solution of anti-sIL7R antibodies. The antibodies bind to and neutralize sIL7R (or reduce its activity) without inhibiting mIL7R. The patient returns to the clinic for evaluation one week after the injection.

The patient reports that her symptoms decreased approximately 80% within 36 hours. Soon thereafter, the patient became asymptomatic (i.e., in remission). The patient is advised to report any signs/symptoms to the healthcare provider. If symptoms return, the patient can be treated with a second injection of the antibodies.

### Example 8

### Administration of Monoclonal Antibody to Treat an Autoimmune Disease

A monoclonal antibody that neutralizes sIL7R is administered to a patient suffering from one of the following autoimmune diseases: achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, Fibromyalgia, Fibrosing alveolitis, Giant cell arteritis (temporal arteritis), Giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), hypogammalglobulinemia, IgA nephropathy, lgG4-related sclerosing disease, Immune thrombocytopenia purpura (ITP), Inclusion body myositis (IBM), Interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myelin oligodendrocyte glycoprotein antibody disorder, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cholangitis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopeni purpura (TTP), thyroid eye disease (TED), Tolosa-Hunt syndrome (THS), transverse myelitis, type 1 diabetes, ulcerative colitis (UC), undifferentiated connective tissue disease (UCTD), uveitis, vasculitis, vitiligo or Vogt-Koyanagi-Harada disease.

Following administration of the monoclonal antibody, the patient suffering from the autoimmune disease begins to recover and is found to have a reduced level of the autoimmune disease.

The therapeutic methods described herein can include the step of administering drug product (i.e., therapeutic) at a pharmaceutically effective amount. The total dose should be determined through appropriate medical judgment by a physician and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, sex, diet, the time and route of administration, the secretion rate of the composition, the time period of therapy, other drugs used in combination or coincident with the composition disclosed herein, and like factors well known in the medical arts.

In still another aspect, the present disclosure provides a use of the therapeutic protein or the pharmaceutical composition including the same in the preparation of drugs for the prevention or treatment of autoimmune diseases and disorders.

In one embodiment, the dose of the composition may be administered hourly, daily, twice daily, thrice daily, four times daily, five times daily, six times daily, seven times daily, eight times daily, nine times daily, ten times daily, semi-weekly, weekly, bi-weekly, tri-weekly or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg/kg, at least 0.25 mg/kg, at least 0.3 mg/kg, at least 0.5 mg/kg, at least 0.75 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, or at least 30 mg/kg. In one embodiment, a weekly dose may be at most 1.5 mg/kg, at most 2 mg/kg, at most 2.5 mg/kg, at most 3 mg/kg, at most 4 mg/kg, at most 5 mg/kg, at most 6 mg/kg, at most 7 mg/kg, at most 8 mg/kg, at most 9 mg/kg, at most 10 mg/kg, at most 15 mg/kg, at most 20 mg/kg, at most 25 mg/kg, or at most 30 mg/kg. In a particular aspect, the weekly dose may range from 5 mg/kg to 20 mg/kg. In an alternative aspect, the weekly dose may range from 10 mg/kg to 15 mg/kg. In another embodiment, the dose ranges from a weekly dose of about 0.01 mg/kg, about 0.25 mg/kg, about 0.3 mg/kg, about 0.5 mg/kg, about 0.75 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 40 mg/kg, about 45 mg/kg or about 50 mg/kg or more.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, sex, drug sensitivity, administration route, administration mode, administration frequency, duration of treatment, drugs used in combination or coincident with the composition disclosed herein, and other factors known in medicine.

The pharmaceutical composition including the therapeutic disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations and compositions include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

The composition may be formulated into a single dosage form suitable for the patient's body, and may be formulated into a preparation useful for peptide drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, sex, gender, body weight, and disease severity.

The total effective dose of the compositions disclosed herein may be administered to a patient in a single dose or may be administered for a long period of time in multiple doses according to a fractionated treatment protocol. In the pharmaceutical composition disclosed herein, the content of active ingredient may vary depending on the disease severity. Preferably, the total daily dose of the peptide disclosed herein may be approximately 0.0001 μg to 500 mg per 1 kg of body weight of a patient. However, the effective dose of the composition is determined considering various factors including patient's age, body weight, health conditions, gender, sex, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical composition may be administered alone or in combination or coincident with other pharmaceutical formulations showing prophylactic or therapeutic efficacy.

Given the teachings and guidance provided herein, those skilled in the art will understand that a formulation or composition described herein can be equally applicable to many types of biopharmaceuticals, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, non-reducing sugars exhibit favorable excipient properties when used with polypeptide biopharmaceuticals compared to reducing sugars. Accordingly, exemplary formulations are exemplified further herein with reference to polypeptide biopharmaceuticals. However, the range of applicability, chemical and physical properties, considerations and methodology applied to polypeptide biopharmaceutical can be similarly applicable to biopharmaceuticals other than polypeptide biopharmaceuticals.

In various embodiments, a formulation or composition can include, without limitation, combinations of bioactive agents (such as viruses, proteins, antibodies, antibody fragments, peptides and the like as described herein) in the formulation. For example, a formulation or composition as described herein can include a single bioactive agent for treatment of one or more conditions. A formulation or composition as described herein also can include, in an embodiment, two or more different bioactive agents for a single or multiple conditions. Use of multiple bioactive agents in a formulation can be directed to, for example, the same or different indications. Similarly, in another embodiment, multiple bioactive agents can be used in a formulation or composition to treat, for example, both a pathological condition and one or more side effects caused by the primary treatment. In a further embodiment, multiple bioactive agents also can be included, in a formulation or composition as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

In various embodiments, a formulation or composition can include one or more preservatives and/or additives known in the art. Similarly, a formulation or composition can further be formulated, without limitation, into any of various known delivery formulations. For example, in an embodiment, a formulation can include: surfactants, adjuvant, biodegradable polymers, hydrogels, etc., such optional components, their chemical and functional characteristics are known in the art. Similarly known in the art are formulations that facilitate rapid, sustained or delayed release of the bioactive agents after administration. A formulation as described can be produced to include these or other formulation components known in the art.

The formulation or composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations or compositions described herein can, without limitation, be administered to patients throughout an extended time period, such as chronic administration for a chronic condition. The composition can be a solid, a semi-solid or an aerosol and a pharmaceutical compositions is formulated as a tablet, geltab, lozenge, orally dissolved strip, capsule, syrup, oral suspension, emulsion, granule, sprinkle or pellet.

The doses of the therapeutic can be expressed in mg per injection such as from about 0.001 mg to 0.5 mg per injection, about 0.01 mg to about 0.5 mg per injection, or about 0.005 mg to about 0.1 mg, or about 0.001 mg to about 5 mg per injection, or about 0.01 to about 5 mg per injection, or about 0.005 mg to about 1 mg per injection, or about 0.005 to about 2 mg per injection, or about 0.005 to 3 mg per injection, or about 0.005 to about 5 mg per injection, or about 0.001 to about 10 mg per injection, or about 0.5 to about 5 mg per injection, or about 0.5 to about 10 mg per injection, or about 0.5 to about 1 mg per injection, or about 0.005 mg, about 0.05 mg, about 0.5 mg, about 0.75 mg, about 1 mg, about 1.25 mg, about 1.5 mg, about 1.75 mg, about 2 mg, about 3 mg, about 4 mg, about 5 mg, about 6 mg, about 7 mg, about 8 mg, about 9 mg or about 10 mg per injection.

The doses of the therapeutic can be expressed in units that are administered. For example, the therapeutic can have a specific activity of about 1,000 units/mg to about 2,500 units/mg, or about 1,500 units/mg, or about 1,750 units/mg, or about 2,000 units/mg, or about 2,500 units/mg, or about 1,000 units/0.58 mg, or 1,724 units/mg wherein “mg” refers to the amount of units present in a composition (as distinct from excipients and other constituents). Accordingly, the present invention contemplates injecting about 100 units to about 500 units per treatment session, or about 1,000 units to about 2,500 units per treatment session.

In another embodiment, the dose of therapeutic per injection is about 50 units to about 2,500 units, or about 85 units to about 2,000 units, or about 150 units to about 1,750 units, or about 200 units to about 1,500 units, or about 300 units to about 1,250 units, or about 500 units to about 1,000 units.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices for use with the pharmaceutical composition and therapeutics of the present invention include pen injectors, auto injectors, safety syringes, injection pumps, infusion pumps, glass prefilled syringes, plastic prefilled syringes and needle free injectors. Syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, California, U.S.A. Administration by injection may be, without limitation intravenous, intramuscular, intraperitoneal, or subcutaneous, as appropriate. Administrations by non-injection route may be, without limitation, nasal, oral, ocular, dermal, or pulmonary, as appropriate.

In certain embodiments, kits can comprise, without limitation, one or more single or multi-chambered syringes (e.g., liquid syringes and lyosyringes) for administering one or more formulations and compositions described herein. In various embodiments, the kit can comprise formulation components for parenteral, subcutaneous, intramuscular or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide compositions in accordance therewith. In various embodiments the kits can contain a lyophilate and a sterile diluent for reconstituting the lyophilate.

Also described herein, are methods for treating a subject in need of therapy, comprising administering to the subject an effective amount of a formulation or composition as described herein. The therapeutically effective amount or dose of a formulation will depend on the disease or condition of the subject and actual clinical setting.

In an embodiment, a composition as described herein can be administered by any suitable route, specifically by parental (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

The compositions as described herein can be used in the manufacture of medicaments and for the treatment of humans and other animals by administration in accordance with conventional procedures.

Also provided herein are combinatorial methods for developing suitable compositions using combinations of amino acids. These methods are effective for developing stable liquid or lyophilized formulations, and particularly pharmaceutical formulations.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical composition or formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75 or 14.

In an embodiment, the pH of the pharmaceutical composition or formulation is from about 3 to about 9, about 4 to about 9, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In embodiments, the anti-sIL7R antibody includes a heavy chain selected from SEQ ID NO: 6, 9, 13, 17, 21, 25, 29, 33, 37 and 41 and a light chain selected from SEQ ID NO: 7, 11, 15, 19, 23, 27, 31, 35, 29 and 43.

In embodiments, the anti-sIL7R antibody includes (1) a heavy chain containing at least the amino acid sequence as set forth in SEQ ID NO: 5, and light chain containing at least the amino acid sequence as set forth in SEQ ID NO: 7; (2) a heavy chain containing at least the amino acid sequence as set forth in SEQ ID NO: 9, and light chain containing at least the amino acid sequence as set forth in SEQ ID NO: 11; (3) a heavy chain containing at least the amino acid sequence as set forth in SEQ ID NO: 13, and light chain containing at least the amino acid sequence as set forth in SEQ ID NO: 15; (4) a heavy chain containing at least the amino acid sequence as set forth in SEQ ID NO: 17, and light chain containing at least the amino acid sequence as set forth in SEQ ID NO: 19; (5) a heavy chain containing at least the amino acid sequence as set forth in SEQ ID NO: 21, and light chain containing at least the amino acid sequence as set forth in SEQ ID NO: 23; (6) a heavy chain containing at least the amino acid sequence as set forth in SEQ ID NO: 25, and light chain containing at least the amino acid sequence as set forth in SEQ ID NO: 27; (7) a heavy chain containing at least the amino acid sequence as set forth in SEQ ID NO: 29, and light chain containing at least the amino acid sequence as set forth in SEQ ID NO: 31; (8) a heavy chain containing at least the amino acid sequence as set forth in SEQ ID NO: 33, and light chain containing at least the amino acid sequence as set forth in SEQ ID NO: 35; (9) a heavy chain containing at least the amino acid sequence as set forth in SEQ ID NO: 37, and light chain containing at least the amino acid sequence as set forth in SEQ ID NO: 39; or (10) a heavy chain containing at least the amino acid sequence as set forth in SEQ ID NO: 41, and light chain containing at least the amino acid sequence as set forth in SEQ ID NO: 43.

In embodiments, the anti-sIL7R antibody includes:
(1) an antibody comprising a heavy chain variable region comprising heavy chain CDR1 containing at least the amino acid sequence as set forth in SEQ ID NO: 44, heavy chain CDR2 containing at least the amino acid sequence as set forth in SEQ ID NO: 45, and heavy chain CDR3 containing at least the amino acid sequence as set forth in SEQ ID NO: 46, and a light chain variable region comprising light chain CDR1 containing at least the amino acid sequence as set forth in SEQ ID NO: 47, light chain CDR2 containing at least the amino acid sequence as set forth in SEQ ID NO: 48, and light chain CDR3 containing at least the amino acid sequence as set forth in SEQ ID NO: 49;
(2) an antibody comprising a heavy chain variable region comprising heavy chain CDR1 containing at least the amino acid sequence as set forth in SEQ ID NO: 50, heavy chain CDR2 containing at least the amino acid sequence as set forth in SEQ ID NO: 51, and heavy chain CDR3 containing at least the amino acid sequence as set forth in SEQ ID NO: 52, and a light chain variable region comprising light chain CDR1 containing at least the amino acid sequence as set forth in SEQ ID NO: 53, light chain CDR2 containing at least the amino acid sequence as set forth in SEQ ID NO: 54, and light chain CDR3 containing at least the amino acid sequence as set forth in SEQ ID NO: 55;
(3) an antibody comprising a heavy chain variable region comprising heavy chain CDR1 containing at least the amino acid sequence as set forth in SEQ ID NO: 56, heavy chain CDR2 containing at least the amino acid sequence as set forth in SEQ ID NO: 57, and heavy chain CDR3 containing at least the amino acid sequence as set forth in SEQ ID NO: 58, and a light chain variable region comprising light chain CDR1 containing at least the amino acid sequence as set forth in SEQ ID NO: 59, light chain CDR2 containing at least the amino acid sequence as set forth in SEQ ID NO: 60, and light chain CDR3 containing at least the amino acid sequence as set forth in SEQ ID NO: 61;
(4) an antibody comprising a heavy chain variable region comprising heavy chain CDR1 containing at least the amino acid sequence as set forth in SEQ ID NO: 62, heavy chain CDR2 containing at least the amino acid sequence as set forth in SEQ ID NO: 63, and heavy chain CDR3 containing at least the amino acid sequence as set forth in SEQ ID NO: 64, and a light chain variable region comprising light chain CDR1 containing at least the amino acid sequence as set forth in SEQ ID NO: 65, light chain CDR2 containing at least the amino acid sequence as set forth in SEQ ID NO: 66, and light chain CDR3 containing at least the amino acid sequence as set forth in SEQ ID NO: 67;
(5) an antibody comprising a heavy chain variable region comprising heavy chain CDR1 containing at least the amino acid sequence as set forth in SEQ ID NO: 68, heavy chain CDR2 containing at least the amino acid sequence as set forth in SEQ ID NO: 69, and heavy chain CDR3 containing at least the amino acid sequence as set forth in SEQ ID NO: 70, and a light chain variable region comprising light chain CDR1 containing at least the amino acid sequence as set forth in SEQ ID NO: 71, light chain CDR2 containing at least the amino acid sequence as set forth in SEQ ID NO: 72, and light chain CDR3 containing at least the amino acid sequence as set forth in SEQ ID NO: 73;
(6) an antibody comprising a heavy chain variable region comprising heavy chain CDR1 containing at least the amino acid sequence as set forth in SEQ ID NO: 74, heavy chain CDR2 containing at least the amino acid sequence as set forth in SEQ ID NO: 75, and heavy chain CDR3 containing at least the amino acid sequence as set forth in SEQ ID NO: 76, and a light chain variable region comprising light chain CDR1 containing at least the amino acid sequence as set forth in SEQ ID NO: 77, light chain CDR2 containing at least the amino acid sequence as set forth in SEQ ID NO: 78, and light chain CDR3 containing at least the amino acid sequence as set forth in SEQ ID NO: 79;
(7) an antibody comprising a heavy chain variable region comprising heavy chain CDR1 containing at least the amino acid sequence as set forth in SEQ ID NO: 80, heavy chain CDR2 containing at least the amino acid sequence as set forth in SEQ ID NO: 81, and heavy chain CDR3 containing at least the amino acid sequence as set forth in SEQ ID NO: 82, and a light chain variable region comprising light chain CDR1 containing at least the amino acid sequence as set forth in SEQ ID NO: 83, light chain CDR2 containing at least the amino acid sequence as set forth in SEQ ID NO: 84, and light chain CDR3 containing at least the amino acid sequence as set forth in SEQ ID NO: 85;
(8) an antibody comprising a heavy chain variable region comprising heavy chain CDR1 containing at least the amino acid sequence as set forth in SEQ ID NO: 86, heavy chain CDR2 containing at least the amino acid sequence as set forth in SEQ ID NO: 87, and heavy chain CDR3 containing at least the amino acid sequence as set forth in SEQ ID NO: 88, and a light chain variable region comprising light chain CDR1 containing at least the amino acid sequence as set forth in SEQ ID NO: 89, light chain CDR2 containing at least the amino acid sequence as set forth in SEQ ID NO: 90, and light chain CDR3 containing at least the amino acid sequence as set forth in SEQ ID NO: 91; or
(9) an antibody comprising a heavy chain variable region comprising heavy chain CDR1 containing at least the amino acid sequence as set forth in SEQ ID NO: 92, heavy chain CDR2 containing at least the amino acid sequence as set forth in SEQ ID NO: 93, and heavy chain CDR3 containing at least the amino acid sequence as set forth in SEQ ID NO: 94, and a light chain variable region comprising light chain CDR1 containing at least the amino acid sequence as set forth in SEQ ID NO: 95, light chain CDR2 containing at least the amino acid sequence as set forth in SEQ ID NO: 96, and light chain CDR3 containing at least the amino acid sequence as set forth in SEQ ID NO: 97;

Provided below are sequence listings (SEQ ID NO: 1-SEQ ID NO: 100). Complementarity-Determining Regions (CDR) within the sequences are bolded and underlined. These CDRs are useful to bind to sIL7R in vitro and in vivo. The CDRs as well as the antibodies defined in the sequences are also useful for treatment of the diseases disclosed herein or for diagnostic uses as also disclosed herein. The CDRs as disclosed can be mixed and matched with other CDRs identified in other SEQ ID NOs. For instance, CDR 1 from SEQ ID NO: 5 could be combined with CDR 2 from SEQ ID NO: 9 and CDR 3 from SEQ ID NO: 7. The first CDR sequence that is bolded and underlined comprises CDR No. 1, the second sequence that is bolded and underlined comprises CDR No. 2, and the third sequence that is bolded and underlined comprises CDR No. 3 within a specific sequence as identified by a SEQ ID NO.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

TABLE 1
mAb amino acid sequences

| Clone ID No. | Heavy Chain SEQ ID NO: | Light Chain SEQ ID NO: |
|---|---|---|
| 2024 | 5 | 7 |
| 2096 | 9 | 11 |
| 2005 | 13 | 15 |
| 2008 | 17 | 19 |
| 2014 | 21 | 23 |
| 2102 | 25 | 27 |
| 2022 | 29 | 31 |
| 2018 | 33 | 35 |
| 2028 | 37 | 39 |
| 2045 | 41 | 43 |

SEQUENCE LISTINGS

SEQ ID NO: 1 (Amino Acid Sequence of sIL7R C-terminal Tail)
LSLSYGPVSPIIRRLWNIFVRNQEKI
SEQ ID NO: 2 (Amino Acid Sequence of sIL7R Region Involved in Binding IL7-not
specific to SIL7R)
FSCYSQLEVNGSQHSLTCAFEDPDVNITNLEFEICGALVEVKCLNFRKLQEIYFIETKKFLLIGKS
NICVKVGEKSLTCKKIDLTTIVKPEAPFDLSVVYREGANDFVVTFNTSHLQKKYVKVLMHDVAYR
QEKDENKWTHVNLSSTKLTLLQRKLQPAAMYEIKVRSIPDHYFKGFWSEWSPSYYFR
SEQ ID NO: 3 (Amino Acid Sequence of sIL7R at the Junction Between the IL7 Binding
Region and the C-Terminal Tail Region)
PDHYFKGFWSEWSPSYYFRTPEINNSSGLSLSYGPVSPIIRRLWNIFVRNQEKI
SEQ ID NO: 4 (Anti-sIL7R mAB Clone ABS-2024 Heavy Chain DNA Sequence)
ATGGGTTGGCTGTGGAACTTGCTATTCCTGATGGCAGCTGCCCAAAGTGCCCAAGCACAG
ATCCAGTTGGTACAGTCTGGACCTGAGCTGAAGAAGCCTGGAGAGACAGTCAGGATCTCC
TGCAAGGCTTCTGGGTATACCTTCACAACCTATGGAATGAGCTGGGTGAAACAGGCTCCA
GGAAAGGGTTTAAAGTGGATGGGCTGTATAAACACCTACTCTGGAGTGCCAACATATCCT
GATGACTTCAAGGGACGGTTTGCCTTCTCTTTGGAAACCTCTGCCAGCACTGCCTATTTGC
AGATCAACAACCTCAAAAATGAGGACACGGCTACATATTTCTGTGCAAGAGAGAAGAATTA
TTACGGTGCTAGGTACTACTTTGACTTCTGGGGCCAAGGCACCACTCTCACAGTCTCCTCA
GCCAAAACAACACCCCCATCAGTCTATCCACTGGCCCCTGGGTGTGGAGATACAACTGGT
TCCTCTGTGACTCTGGGATGCCTGGTCAAGGGCTACTTCCCTGAGTCAGTGACTGTGACTT
GGAACTCTGGATCCCTGTCCAGCAGTGTGCACACCTTCCCAGCTCTCCTGCAGTCTGGAC
TCTACACTATGAGCAGCTCAGTGACTGTCCCCTCCAGCACCTGGCCAAGTCAGACCGTCA
CCTGCAGCGTTGCTCACCCAGCCAGCAGCACCACGGTGGACAAAAAACTTGAGCCCAGCG
GGCCCATTTCAACAATCAACCCCTGTCCTCCATGCAAGGAGTGTCACAAATGCCCAGCTCC
TAACCTCGAGGGTGGACCATCCGTCTTCATCTTCCCTCCAAATATCAAGGATGTACTCATG
ATCTCCCTGACACCCAAGGTCACGTGTGTGGTGGTGGATGTGAGCGAGGATGACCCAGAC
GTCCGGATCAGCTGGTTTGTGAACAACGTGGAAGTACACACAGCTCAGACACAAACCCATA
GAGAGGATTACAACAGTACTATCCGGGTGGTCAGTGCCCTCCCCATCCAGCACCAGGACT
GGATGAGTGGCAAGGAGTTCAAATGCAAGGTCAACAACAAAGACCTCCCATCACCCATCG
AGAGAACCATCTCAAAAATTAAAGGGCTAGTCAGAGCTCCACAAGTATACATCTTGCCGCC
ACCAGCAGAGCAGTTGTCCAGGAAAGATGTCAGTCTCACTTGCCTGGTCGTGGGCTTC
AACCCTGGAGACATCAGTGTGGAGTGGACCAGCAATGGGCATACAGAGGAGAACTACAAG
GACACCGCACCAGTCCTGGACTCTGACGGTTCTTACTTCATATACAGCAAGCTCGATATAA
AAACAAGCAAGTGGGAGAAAACAGATTCCTTCTCATGCAACGTGAGACACGAGGGTCTGA
AAAATTACTACCTGAAGAAG ACCATCTCCCGGTCTCCGGGTAAATGA
SEQ ID NO: 5 (Anti-sIL7R mAB Clone ABS-2024 Heavy Chain Amino Acid Sequence)
MGWLWNLLFLMAAAQSAQAQIQLVQSGPELKKPGETVRISCKASGYTFTTYGMSWVKQAPGK
GLKWMGCINTYSGVPTYPDDFKGRFAFSLETSASTAYLQINNLKNEDTATYFCAREKNYYGAR
YYFDFWGQGTTLTVSSAKTTPPSVYPLAPGCGDTTGSSVTLGCLVKGYFPESVTVTWNSGSL
SSSVHTFPALLQSGLYTMSSSVTVPSSTWPSQTVTCSVAHPASSTTVDKKLEPSGPISTINPCP
PCKECHKCPAPNLEGGPSVFIFPPNIKDVLMISLTPKVTCVVVDVSEDDPDVRISWFVNNVEVH
TAQTQTHREDYNSTIRVVSALPIQHQDWMSGKEFKCKVNNKDLPSPIERTISKIKGLVRAPQVYI
LPPPAEQLSRKDVSLTCLVVGFNPGDISVEWTSNGHTEENYKDTAPVLDSDGSYFIYSKLDIKT
SKWEKTDSFSCNVRHEGLKNYYLKKTISRSPGK
SEQ ID NO: 6 (Anti-sIL7R mAB Clone ABS-2024 Light Chain DNA Sequence)
ATGAAGTTGCCTGTTAGGCTGTTGGTGCTGATGTTCTGGATTCCTGCTTCCAGCAGTGATG
TTGTGATGACCCAAACTCCACTCTCCCTGCCTGTCAGTCTTGGAGATCAAGCCTCCATCTC
TTGCAGATCTAGTCAGAGCATTGTACACAGTTATAGATACACCTATTTAGAATGGTACCT
GCAGAAACCAGGCCAGTCACTAAAGCTCCTGATATATGGGGTTTCCAACCGATTTTCTGGG
GTCCCAGACAGGTTCAGTGGCAGTGGATCAGGGACAGATTTCACACTCAAGATCAGCAGA
GTGGAGGCTGAGGATATGGGAGTTTATTACTGCTTTCAAGGTACACATGTTCCGCTCACG
TTCGGTGCTGGGACCAAGCTGGAGCTGAAACGGGCTGATGCTGCACCAACTGTATCCATC
TTCCCACCATCCAGTGAGCAGTTAACATCTGGAGGTGCCTCAGTCGTGTGCTTCTTGAACA
ACTTCTACCCCAGAGACATCAATGTCAAGTGGAAGATTGATGGCAGTGAACGACAAAATGG
TGTCCTGAACAGTTGGACTGATCAGGACAGCAAAGACAGCACCTACAGCATGAGCAGCAC
CCTCACATTGACCAAGGACGAGTATGAACGACATAACAGCTATACCTGTGAGGCCACTCAC
AAGACATCAACTTCACCCATCGTCAAGAGCTTCAACAGGAATGAGTGTTAG
SEQ ID NO: 7 (Anti-sIL7R mAB Clone ABS-2024 Light Chain Amino Acid Sequence)
MKLPVRLLVLMFWIPASSSDVVMTQTPLSLPVSLGDQASISCRSSQSIVHSYRYTYLEWYLQKP
GQSLKLLIYGVSNRFSGVPDRFSGSGSGTDFTLKISRVEAEDMGVYYCFQGTHVPLTFGAGTK
LELKRADAAPTVSIFPPSSEQLTSGGASVVCFLNNFYPRDINVKWKIDGSERQNGVLNSWTDD
SKDSTYSMSSTLTLTKDEYERHNSYTCEATHKTSTSPIVKSFNRNEC
SEQ ID NO: 8 (Anti-sIL7R mAB Clone ABS-2096 Heavy Chain DNA Sequence)
ATGGGCAGGCTTACTTCTTCATTCCTGCTACTGATTGTCCCTGCATATGTCCTGTCCCAGG
TCACTCTGAAAGAGTCTGGCCCTGGGATATTGCAGCCCTCCCAGACCCTCAGTCTGACTTG
TTCTTTCTCTGGGTTTTCACTGAGCACTTATGGTATGGGTGTAGGTTGGATTCGTCAGCCT
TCAGGGAAGGGTCTGGAGTGGCTGGCCAACATTTGGTGGAATGATTATAAATACTATAAC
TCAGCCCTGAAGAGCCGGCTCACAATCTCCAAGGATCCCCCCAACAACCAGGTATTCCTC
AAGATCTCCAGTGTGGACACTGCAGATACTGCCACATACTACTGTGCTCAAGTAGGAAACT
ACGATGGTGGCTTCCACTACTGGGGCCAAGGCACCACTCTCACAGTCTCCTCAGCCAAAA
CGACACCCCCATCTGTCTATCCACTGGCCCCTGGATCTGCTGCCCAAACTAACTCCATGGT
GACCCTGGGATGCCTGGTCAAGGGCTATTTCCCTGAGCCAGTGACAGTGACCTGGAACTC
TGGATCCCTGTCCAGCGGTGTGCACACCTTCCCAGCTGTCCTGCAGTCTGACCTCTACACT
CTGAGCAGCTCAGTGACTGTCCCCTCCAGCACCTGGCCCAGCGAGACCGTCACCTGCAAC
GTTGCCCACCCGGCCAGCAGCACCAAGGTGGACAAGAAAATTGTGCCCAGGGATTGTGGT
TGTAAGCCTTGCATATGTACAGTCCCAGAAGTATCATCTGTCTTCATCTTCCCCCCAAAGCC
CAAGGATGTGCTCACCATTACTCTGACTCCTAAGGTCACGTGTGTTGTGGTAGACATCAGC
AAGGATGATCCCGAGGTCCAGTTCAGCTGGTTTGTAGATGATGTGGAGGTGCACACAGCT
CAGACGCAACCCCGGGAGGAGCAGTTCAACAGCACTTTCCGCTCAGTCAGTGAACTTCCC
ATCATGCACCAGGACTGGCTCAATGGCAAGGAGTTCAAATGCAGGGTCAACAGTGCAGCT
TTCCCTGCCCCCATCGAGAAAACCATCTCCAAAACCAAAGGCAGACCGAAGGCTCCACAG
GTGTACACCATTCCACCTCCCAAGGAGCAGATGGCCAAGGATAAAGTCAGTCTGACCTGC
ATGATAACAGACTTCTTCCCTGAAGACATTACTGTGGAGTGGCAGTGGAATGGGCAGCCA
GCGGAGAACTACAAGAACACTCAGCCCATCATGGACACAGATGGCTCTTACTTCATCTACA
GCAAGCTCAATGTGCAGAAGAGCAACTGGGAGGCAGGAAATACTTTCACCTGCTCTGTGTT
ACATGAGGGCCTGCACAACCACCATACTGAGAAGAGCCTCTCCCACTCTCCTGGTAAATGA
SEQ ID NO: 9 (Anti-sIL7R mAB Clone ABS-2096 Heavy Chain Amino Acid Sequence)
MGRLTSSFLLLIVPAYVLSQVTLKESGPGILQPSQTLSLTCSFSGFSLSTYGMGVGWIRQPSGK
GLEWLANIWWNDYKYYNSALKSRLTISKDPPNNQVFLKISSVDTADTATYYCAQVGNYDGGFH
YWGQGTTLTVSSAKTTPPSVYPLAPGSAAQTNSMVTLGCLVKGYFPEPVTVTWNSGSLSSGV
HTFPAVLQSDLYTLSSSVTVPSSTWPSETVTCNVAHPASSTKVDKKIVPRDCGCKPCICTVPEV
SSVFIFPPKPKDVLTITLTPKVTCVVVDISKDDPEVQFSWFVDDVEVHTAQTQPREEQFNSTFR
SVSELPIMHQDWLNGKEFKCRVNSAAFPAPIEKTISKTKGRPKAPQVYTIPPPKEQMAKDKVSL
TCMITDFFPEDITVEWQWNGQPAENYKNTQPIMDTDGSYFIYSKLNVQKSNWEAGNTFTCSVL
HEGLHNHHTEKSLSHSPGK
SEQ ID NO: 10 (Anti-sIL7R mAB Clone ABS-2096 Light Chain DNA Sequence)
ATGAAGTTGCCTGTTAGGCTGTTGGTGCTGATGTTCTGGATTCCTGCTTCCAGCAGTGATG
TTGTGATGACCCAAACTCCACTCTCCCTGCCTGTCAGTCTTGGAGATCAAGCCTCCATCTC
TTGCAGATCTAGTCAGACCCTTGTACACAGTAATGGAAACACCTATTTACATTGGTACCT
GCAGAGGCCAGGCCAGTCTCCAAAGCTCCTGATCTACAAAGTTTCCAACCGATTTTCTGG
GGTCCCAGACAGGTTCAGTGGCAGTGGATCAGGGACAGATTTCACACTCAAGATCAGCAG
AGTGGAGGCTGAGGATCTGGGAGTTTATTTCTGCTCTCAAAGTACACATGTTCCGTGGAC
GTTCGGTGGAGGCACCAAGCTGGAAATCAAACGGGCTGATGCTGCACCAACTGTATCCAT
CTTCCCACCATCCAGTGAGCAGTTAACATCTGGAGGTGCCTCAGTCGTGTGCTTCTTGAAC
AACTTCTACCCCAAAGACATCAATGTCAAGTGGAAGATTGATGGCAGTGAACGACAAAATG
GCGTCCTGAACAGTTGGACTGATCAGGACAGCAAAGACAGCACCTACAGCATGAGCAGCA
CCCTCACGTTGACCAAGGACGAGTATGAACGACATAACAGCTATACCTGTGAGGCCACTCA
CAAGA CATCAACTTCACCCATTGTCAAGAGCTTCAACAGGAATGAGTGTTAG
SEQ ID NO: 11 (Anti-sIL7R mAB Clone ABS-2096 Light Chain Amino Acid Sequence)
MKLPVRLLVLMFWIPASSSDVVMTQTPLSLPVSLGDQASISCRSSQTLVHSNGNTYLHWYLQR
PGQSPKLLIYKVSNRFSGVPDRFSGSGSGTDFTLKISRVEAEDLGVYFCSQSTHVPWTFGGGT
KLEIKRADAAPTVSIFPPSSEQLTSGGASVVCFLNNFYPKDINVKWKIDGSERQNGVLNSWTDQ
DSKDSTYSMSS TLTLTKDEYERHNSYTCEATHKTSTSPIVKSFNRNEC
SEQ ID NO: 12 (Anti-sIL7R mAB Clone ABS-2005 Heavy Chain DNA Sequence)
ATGGGTTGGCTGTGGAACTTGCTATTCCTGATGGCAGCTGCCCAAAGTGCCCAAGCACAG
ATCCAGTTGGTACAGTCTGGACCTAAACTGAAGAAGCCTGGAGAGACAGTCAAGATCTCCT
GCAGGGCTTCTGGGTATCCCTTCACAACCTATGGAATGAACTGGGTGAAACAGGCTCCAG
GAAAGGGTTTAAAGTGGATGGGTTGGATAAATACCTACTCTGGAGTGCCAACATATGTTG
ATGACTTCAAGGGACGGTTTGCCTTCTCTTTGGAAACCTCTGCCACCACTGCCCATTTGCA
GATCAACAACCTCAAAAATGAGGACACGGCTACATATTTCTGTGCAAGATGGGGTACTCCC
AGGTATTTCTTTGACTATTGGGGCCAAGGCACCACTCTCACAGTCTCCTCAGCCAAAACAA
CACCCCCATCAGTCTATCCACTGGCCCCTGGGTGTGGAGATACAACTGGTTCCTCTGTGA
CTCTGGGATGCCTGGTCAAGGGCTACTTCCCTGAGTCAGTGACTGTGACTTGGAACTCTG
GATCCCTGTCCAGCAGTGTGCACACCTTCCCAGCTCTCCTGCAGTCTGGACTCTACACTAT
GAGCAGCTCAGTGACTGTCCCCTCCAGCACCTGGCCAAGTCAGACCGTCACCTGCAGCGT
TGCTCACCCAGCCAGCAGCACCACGGTGGACAAAAAACTTGAGCCCAGCGGGCCCATTTC
AACAATCAACCCCTGTCCTCCATGCAAGGAGTGTCACAAATGCCCAGCTCCTAACCTCGAG
GGTGGACCATCCGTCTTCATCTTCCCTCCAAATATCAAGGATGTACTCATGATCTCCCTGA
CACCCAAGGTCACGTGTGTGGTGGTGGATGTGAGCGAGGATGACCCAGACGTCCGGATC
AGCTGGTTTGTGAACAACGTGGAAGTACACACAGCTCAGACACAAACCCATAGAGAGGATT
ACAACAGTACTATCCGGGTGGTCAGTGCCCTCCCCATCCAGCACCAGGACTGGATGAGTG
GCAAGGAGTTCAAATGCAAGGTCAACAACAAAGACCTCCCATCACCCATCGAGAGAACCAT
CTCAAAAATTAAAGGGCTAGTCAGAGCTCCACAAGTATACATCTTGCCGCCACCAGCAGAG
CAGTTGTCCAGGAAAGATGTCAGTCTCACTTGCCTGGTCGTGGGCTTCAACCCTGGAGAC
ATCAGTGTGGAGTGGACCAGCAATGGGCATACAGAGGAGAACTACAAGGACACCGCACCA
GTCCTGGACTCTGACGGTTCTTACTTCATATACAGCAAGCTCGATATAAAAACAAGCAAGT
GGGAGAAAACAGATTCCTTCTCATGCAACGTGAGACACGAGGGTCTGAAAAATTACTACCT
GAAGAAGACCATCTCC CGGTCTCCGGGTAAATGA
SEQ ID NO: 13 (Anti-sIL7R mAB Clone ABS-2005 Heavy Chain Amino Acid Sequence)
MGWLWNLLFLMAAAQSAQAQIQLVQSGPKLKKPGETVKISCRASGYPFTTYGMNWVKQAPGK
GLKWMGWINTYSGVPTYVDDFKGRFAFSLETSATTAHLQINNLKNEDTATYFCARWGTPRYFF
DYWGQGTTLTVSSAKTTPPSVYPLAPGCGDTTGSSVTLGCLVKGYFPESVTVTWNSGSLSSS
VHTFPALLQSGLYTMSSSVTVPSSTWPSQTVTCSVAHPASSTTVDKKLEPSGPISTINPCPPCK
ECHKCPAPNLEGGPSVFIFPPNIKDVLMISLTPKVTCVVVDVSEDDPDVRISWFVNNVEVHTAQ
TQTHREDYNSTIRVVSALPIQHQDWMSGKEFKCKVNNKDLPSPIERTISKIKGLVRAPQVYILPP
PAEQLSRKDVSLTCLVVGFNPGDISVEWTSNGHTEENYKDTAPVLDSDGSYFIYSKLDIKTSKW
EKTDSFSCNVRHEGLKNYYLKKTISRSPGK
SEQ ID NO: 14 (Anti-sIL7R mAB Clone ABS-2005 Light Chain DNA Sequence)
ATGAAGTTGCCTGTTAGGCTGTTGGTGCTGATGTTCTGGATTCCTGCTTCCAGCAGTGATG
TTGTGATGACCCAAACTCCACTCTCCCTGCCTGTCAGTCTTGGAGATCAAGCCTCCATCTC
TTGCAGATCTAGTCAGAGCATTGTACACAGTAATAGATACACCTATTTAGAATGGTACCT
GCAGAAACCAGGCCAGTCGCTAAAGCTCCTGATATATGGGGTTTCCAACCGATTTTCTGG
GGTCCCAGACAGGTTCAGTGGCAGTGGATCAGGGACAGATTTCACACTCAAGATCAGCAG
AGTGGAGGCTGAGGATATGGGAGTTTATTACTGCTTTCAAGGTACACATGTTCCGTGGAC
GTTCGGTGGAGGCACCAAGCTGGAAATCAAACGGGCTGATGCTGCACCAACTGTATCCAT
CTTCCCACCATCCAGTGAGCAGTTAACATCTGGAGGTGCCTCAGTCGTGTGCTTCTTGAAC
AACTTCTACCCCAAAGACATCAATGTCAAGTGGAAGATTGATGGCAGTGAACGACAAAATG
GCGTCCTGAACAGTTGGACTGATCAGGACAGCAAAGACAGCACCTACAGCATGAGCAGCA
CCCTCACGTTGACCAAGGACGAGTATGAACGACATAACAGCTATACCTGTGAGGCCACTCA
CAAGA CATCAACTTCACCCATTGTCAAGAGCTTCAACAGGAATGAGTGTTAG
SEQ ID NO: 15 (Anti-sIL7R mAB Clone ABS-2005 Light Chain Amino Acid Sequence)
MKLPVRLLVLMFWIPASSSDVVMTQTPLSLPVSLGDQASISCRSSQSIVHSNRYTYLEWYLQKP
GQSLKLLIYGVSNRFSGVPDRFSGSGSGTDFTLKISRVEAEDMGVYYCFQGTHVPWTFGGGT
KLEIKRADAAPTVSIFPPSSEQLTSGGASVVCFLNNFYPKDINVKWKIDGSERQNGVLNSWTDQ
DSKDSTYSMSS TLTLTKDEYERHNSYTCEATHKTSTSPIVKSFNRNEC
SEQ ID NO: 16 (Anti-sIL7R mAB Clone ABS-2008 Heavy Chain DNA Sequence)
ATGGGTTGGCTGTGGAACTTGCTATTCCTGATGGCAGCTGCCCAAAGTGCCCAAGCACAG
ATCCAGTTGGTACAGTCTGGACCTGAGCTGAAGAAGCCTGGAGAGACAGTCAAGATCTCC
TGCAAGGCCTCTGGGTATACCTTCACAACCTATGGAATGAACTGGGTGCAACAGGCTCCA
GGAAAGGGTTTAAAGTGGATGGGCTGGATAAACACCTACTCTGGAGTGCCAACATATGCT
GATGACTTCAAGGGACGGTTTGCCTTCTCTTTGGAAACCTCTGCCAGCACTGCCTATTTGC
AGATCAACAACCTCAAAAATGAGGACACGGCCAAATATTTCTGTACAAGATCTGGGGGATT
ACGACCCTGGTTTGCTTACTGGGGCCAAGGGACTCTGGTCACTGTCTCTGCAGCCAAAAC
AACACCCCCATCAGTCTATCCACTGGCCCCTGGGTGTGGAGATACAACTGGTTCCTCTGTG
ACTCTGGGATGCCTGGTCAAGGGCTACTTCCCTGAGTCAGTGACTGTGACTTGGAACTCT
GGATCCCTGTCCAGCAGTGTGCACACCTTCCCAGCTCTCCTGCAGTCTGGACTCTACACT
ATGAGCAGCTCAGTGACTGTCCCCTCCAGCACCTGGCCAAGTCAGACCGTCACCTGCAGC
GTTGCTCACCCAGCCAGCAGCACCACGGTGGACAAAAAACTTGAGCCCAGCGGGCCCATT
TCAACAATCAACCCCTGTCCTCCATGCAAGGAGTGTCACAAATGCCCAGCTCCTAACCTCG
AGGGTGGACCATCCGTCTTCATCTTCCCTCCAAATATCAAGGATGTACTCATGATCTCCCT
GACACCCAAGGTCACGTGTGTGGTGGTGGATGTGAGCGAGGATGACCCAGACGTCCGGA
TCAGCTGGTTTGTGAACAACGTGGAAGTACACACAGCTCAGACACAAACCCATAGAGAGG
ATTACAACAGTACTATCCGGGTGGTCAGTGCCCTCCCCATCCAGCACCAGGACTGGATGA
GTGGCAAGGAGTTCAAATGCAAGGTCAACAACAAAGACCTCCCATCACCCATCGAGAGAA
CCATCTCAAAAATTAAAGGGCTAGTCAGAGCTCCACAAGTATACATCTTGCCGCCACCAGC
AGAGCAGTTGTCCAGGAAAGATGTCAGTCTCACTTGCCTGGTCGTGGGCTTCAACCCTGG
AGACATCAGTGTGGAGTGGACCAGCAATGGGCATACAGAGGAGAACTACAAGGACACCGC
ACCAGTCCTGGACTCTGACGGTTCTTACTTCATATACAGCAAGCTCGATATAAAAACAAGCA
AGTGGGAGAAAACAGATTCCTTCTCATGCAACGTGAGACACGAGGGTCTGAAAAATTACTA
CCTGAAGAAGACCATCTCC CGGTCTCCGGGTAAATGA
SEQ ID NO: 17 (Anti-sIL7R mAB Clone ABS-2008 Heavy Chain Amino Acid Sequence)
MGWLWNLLFLMAAAQSAQAQIQLVQSGPELKKPGETVKISCKASGYTFTTYGMNWVQQAPGK
GLKWMGWINTYSGVPTYADDFKGRFAFSLETSASTAYLQINNLKNEDTAKYFCTRSGGLRPW
FAYWGQGTLVTVSAAKTTPPSVYPLAPGCGDTTGSSVTLGCLVKGYFPESVTVTWNSGSLSS
SVHTFPALLQSGLYTMSSSVTVPSSTWPSQTVTCSVAHPASSTTVDKKLEPSGPISTINPCPPC
KECHKCPAPNLEGGPSVFIFPPNIKDVLMISLTPKVTCVVVDVSEDDPDVRISWFVNNVEVHTA
QTQTHREDYNSTIRVVSALPIQHQDWMSGKEFKCKVNNKDLPSPIERTISKIKGLVRAPQVYILP
PPAEQLSRKDVSLTCLVVGFNPGDISVEWTSNGHTEENYKDTAPVLDSDGSYFIYSKLDIKTSK
WEKTDSFSCNVRHEGLKNYYLKKTIS RSPGK
SEQ ID NO: 18 (Anti-sIL7R mAB Clone ABS-2008 Light Chain DNA Sequence)
ATGAGTCCTGCCCAGTTCCTGTTTCTGCTAGTGCTCTCGATTCAGGAAACCAATGGTGATG
TTGTGATGACCCAGACTCCACTGTCTTTGTCGGTTACCATTGGACAACCAGCCTCCATCTC
TTGCAAGTCAAGTCAGAGCCTCTTACATAGTAATGGAAAGACATATTTGAATTGGTTATTA
CAGAGGCCAGGCCAGTCTCCAAAGCTCCTAATCTATCTGGTGTCTAAACTGGAATCTGGC
ATCCCTGACAGGTTCAGTGGCAGTGGATCAGGGACAGATTTCACACTGAAAATCAGCAGA
GTGGAGGTTGAGGATTTGGGAGTTTATTACTGCTTGCAACATACACATTTTCCTCATACGT
TCGGATCGGGGACCAAGCTGGAAATAAAACGGGCTGATGCTGCACCAACTGTATCCATCT
TCCCACCATCCAGTGAGCAGTTAACATCTGGAGGTGCCTCAGTCGTGTGCTTCTTGAACAA
CTTCTACCCCAAAGACATCAATGTCAAGTGGAAGATTGATGGCAGTGAACGACAAAATGGC
GTCCTGAACAGTTGGACTGATCAGGACAGCAAAGACAGCACCTACAGCATGAGCAGCACC
CTCACGTTGACCAAGGACGAGTATGAACGACATAACAGCTATACCTGTGAGGCCACTCACA
AGA CATCAACTTCACCCATTGTCAAGAGCTTCAACAGGAATGAGTGTTAG
SEQ ID NO: 19 (Anti-sIL7R mAB Clone ABS-2008 Light Chain Amino Acid Sequence)
MSPAQFLFLLVLSIQETNGDVVMTQTPLSLSVTIGQPASISCKSSQSLLHSNGKTYLNWLLQRP
GQSPKLLIYLVSKLESGIPDRFSGSGSGTDFTLKISRVEVEDLGVYYCLQHTHFPHTFGSGTKL
EIKRADAAPTVSIFPPSSEQLTSGGASVVCFLNNFYPKDINVKWKIDGSERQNGVLNSWTDQDS
KDSTYSMSS TLTLTKDEYERHNSYTCEATHKTSTSPIVKSFNRNEC
SEQ ID NO: 20 (Anti-sIL7R mAB Clone ABS-2014 Heavy Chain DNA Sequence)
ATGAACTTTGTGCTCAGCTTGATTTTCCTTGCCCTCATTTTAAAAGGTGTCCAGTGTGAAGT
GCAGCTGGTGGAGTCTGGGGGAGGCTCAGTGAAGCCTGGAGGGTCCCTGAAACTCTCCT
GTGCAGCCTCTGGATTCACTTTCAGTCGCTATGCCATGTCTTGGGTTCGCCAGACTCCGG
AGAAGAGGCTGGAGTGGGTCGCAACCATTAATGATGGTGGTAGTTACACCTACTATCCAG
ACAGTGTGAAGGGCCGATTCACCATCTCCAGAGACAATGCCAAGAACACCCTGTACCTGC
AAATGAGCAGTCTGAGGTCTGAGGACACGGCCATATATTACTGTACAAGAGAGGGCTACT
ACGGTTCTTTTGACTACTGGGGCCAAGGCACCACTCTCACAGTCTCCTCAGCCAAAACAAC
ACCCCCATCAGTCTATCCACTGGCCCCTGGGTGTGGAGATACAACTGGTTCCTCTGTGACT
CTGGGATGCCTGGTCAAGGGCTACTTCCCTGAGTCAGTGACTGTGACTTGGAACTCTGGA
TCCCTGTCCAGCAGTGTGCACACCTTCCCAGCTCTCCTGCAGTCTGGACTCTACACTATGA
GCAGCTCAGTGACTGTCCCCTCCAGCACCTGGCCAAGTCAGACCGTCACCTGCAGCGTTG
CTCACCCAGCCAGCAGCACCACGGTGGACAAAAAACTTGAGCCCAGCGGGCCCATTTCAA
CAATCAACCCCTGTCCTCCATGCAAGGAGTGTCACAAATGCCCAGCTCCTAACCTCGAGG
GTGGACCATCCGTCTTCATCTTCCCTCCAAATATCAAGGATGTACTCATGATCTCCCTGACA
CCCAAGGTCACGTGTGTGGTGGTGGATGTGAGCGAGGATGACCCAGACGTCCGGATCAG
CTGGTTTGTGAACAACGTGGAAGTACACACAGCTCAGACACAAACCCATAGAGAGGATTAC
AACAGTACTATCCGGGTGGTCAGTGCCCTCCCCATCCAGCACCAGGACTGGATGAGTGGC
AAGGAGTTCAAATGCAAGGTCAACAACAAAGACCTCCCATCACCCATCGAGAGAACCATCT
CAAAAATTAAAGGGCTAGTCAGAGCTCCACAAGTATACATCTTGCCGCCACCAGCAGAGCA
GTTGTCCAGGAAAGATGTCAGTCTCACTTGCCTGGTCGTGGGCTTCAACCCTGGAGACAT
CAGTGTGGAGTGGACCAGCAATGGGCATACAGAGGAGAACTACAAGGACACCGCACCAGT
CCTGGACTCTGACGGTTCTTACTTCATATACAGCAAGCTCGATATAAAAACAAGCAAGTGG
GAGAAAACAGATTCCTTCTCATGCAACGTGAGACACGAGGGTCTGAAAAATTACTACCTGA
AGAAGACCATCTCCCGG TCTCCGGGTAAATGA
SEQ ID NO: 21 (Anti-sIL7R mAB Clone ABS-2014 Heavy Chain Amino Acid Sequence)
MNFVLSLIFLALILKGVQCEVQLVESGGGSVKPGGSLKLSCAASGFTFSRYAMSWVRQTPEKR
LEWVATINDGGSYTYYPDSVKGRFTISRDNAKNTLYLQMSSLRSEDTAIYYCTREGYYGSFDY
WGQGTTLTVSSAKTTPPSVYPLAPGCGDTTGSSVTLGCLVKGYFPESVTVTWNSGSLSSSVH
TFPALLQSGLYTMSSSVTVPSSTWPSQTVTCSVAHPASSTTVDKKLEPSGPISTINPCPPCKEC
HKCPAPNLEGGPSVFIFPPNIKDVLMISLTPKVTCVVVDVSEDDPDVRISWFVNNVEVHTAQTQ
THREDYNSTIRVVSALPIQHQDWMSGKEFKCKVNNKDLPSPIERTISKIKGLVRAPQVYILPPPA
EQLSRKDVSLTCLVVGFNPGDISVEWTSNGHTEENYKDTAPVLDSDGSYFIYSKLDIKTSKWEK
TDSFSCNVRHEGLKNYYLKKTISR SPGK
SEQ ID NO: 22 (Anti-sIL7R mAB Clone ABS-2014 Light Chain DNA Sequence)
ATGAGTCCTGCCCAGTTCCTGTTTCTGCTAGTGCTCTCGATTCAGGAAATCAACGGTGATG
TTGTGATGACCCAGACTCCACTCACTTTGTCGGTTACCATTGGACAACCAGCTTCCATCTCT
TGCAAGTCAAGTCAGAGCCTCTTATATACTAATGGAAAAACCTATTTGAATTGGTTATTAC
AGAGGCCAGGCCAGTCTCCAAAACGCCTAATCTATCTGGTGTCTAAATTGGACTCTGGAG
TCCCTGACAGGTTCAGTGGCAGTGGATCAGGGACAGATTTCACACTGAAAATCAGCAGAG
TGGAGGCTGAGGATTTGGGAGTTTATTACTGCTTGCAGAGTACACGTTTTCCTCAGACGTT
CGGTGGAGGCACCAAGCTGGAAATCAAACGGGCTGATGCTGCACCAACTGTATCCATCTT
CCCACCATCCAGTGAGCAGTTAACATCTGGAGGTGCCTCAGTCGTGTGCTTCTTGAACAAC
TTCTACCCCAGAGACATCAATGTCAAGTGGAAGATTGATGGCAGTGAACGACAAAATGGTG
TCCTGAACAGTTGGACTGATCAGGACAGCAAAGACAGCACCTACAGCATGAGCAGCACCC
TCACATTGACCAAGGACGAGTATGAACGACATAACAGCTATACCTGTGAGGCCACTCACAA
GA CATCAACTTCACCCATCGTCAAGAGCTTCAACAGGAATGAGTGTTAG
SEQ ID NO: 23 (Anti-sIL7R mAB Clone ABS-2014 Light Chain Amino Acid Sequence)
MSPAQFLFLLVLSIQEINGDVVMTQTPLTLSVTIGQPASISCKSSQSLLYTNGKTYLNWLLQRPG
QSPKRLIYLVSKLDSGVPDRFSGSGSGTDFTLKISRVEAEDLGVYYCLQSTRFPQTFGGGTKL
EIKRADAAPTVSIFPPSSEQLTSGGASVVCFLNNFYPRDINVKWKIDGSERQNGVLNSWTDQD
SKDSTYSMSS TLTLTKDEYERHNSYTCEATHKTSTSPIVKSFNRNEC
SEQ ID NO: 24 (Anti-sIL7R mAB Clone ABS-2102 Heavy Chain DNA Sequence)
ATGGGATGGAACTGGATCTTTATTTTAATCCTGTCAGTAACTACAGGTGTCCACTCTGAGGT
CCAGCTGCAGCAGTCTGGACCTGAGCTGGTGAAGCCTGGGGCTTCAGTGAAGATATCCTG
CAAGGCTTCTGGTTACTCATTCACTGGCTACTACATGCACTGGGTGAAGCAAAGTCCTGAA
AAGAGCCTTGAGTGGATTGGAAAGATTAATCCTTACACTGGTGGTACTACCTACAACCAC
AAGTTCAAGGCCAAGGCCACATTGACTGTAGACAAATCCTCCAGCACAGCCTACATGCAG
CTCAAGAGCCTGACATCTGAGGACTCTGCAGTCTATTACTGTGCAAGAAGTTATTACTACG
CTGGTAGCCCCCCCTTTGACTACTGGGGCCAAGGCACCACTCTCACAGTCTCCTCAGCCA
AAACAACAGCCCCATCGGTCTATCCACTGGCCCCTGTGTGTGGAGATACAAGTGGCTCCT
CGGTGACTCTAGGATGCCTGGTCAAGGGTTATTTCCCTGAGCCAGTGACCTTGACCTGGA
ACTCTGGATCCCTGTCCAGTGGTGTGCACACCTTCCCAGCTGTCCTGCAGTCTGACCTCTA
CACCCTCAGCAGCTCAGTGACTGTAACCTCGAGCACCTGGCCCAGCCAGTCCATCACCTG
CAATGTGGCCCACCCGGCAAGCAGCACCAAGGTGGACAAGAAAATTGAGCCCAGAGGGC
CCACAATCAAGCCCTGTCCTCCATGCAAATGCCCAGCACCTAACCTCTTGGGTGGACCATC
CGTCTTCATCTTCCCTCCAAAGATCAAGGATGTACTCATGATCTCCCTGAGCCCCATAGTC
ACATGTGTGGTGGTGGATGTGAGCGAGGATGACCCAGATGTCCAGATCAGCTGGTTTGTG
AACAACGTGGAAGTACACACAGCTCAGACACAAACCCATAGAGAGGATTACAACAGTACTC
TCCGGGTGGTCAGTGCCCTCCCCATCCAGCACCAGGACTGGATGAGTGGCAAGGAGTTCA
AATGCAAGGTCAACAACAAAGACCTCCCAGCGCCCATCGAGAGAACCATCTCAAAACCCAA
AGGGTCAGTAAGAGCTCCACAGGTATATGTCTTGCCTCCACCAGAAGAAGAGATGACTAAG
AAACAGGTCACTCTGACCTGCATGGTCACAGACTTCATGCCTGAAGACATTTACGTGGAGT
GGACCAACAACGGGAAAACAGAGCTAAACTACAAGAACACTGAACCAGTCCTGGACTCTG
ATGGTTCTTACTTCATGTACAGCAAGCTGAGAGTGGAAAAGAAGAACTGGGTGGAAAGAAA
TAGCTACTCCTGTTCAGTGGTCCACGAGGGTCTGCACAATCACCACACGACTAAGAGCTTC
TCCCGGACTCCGGGTAAATGA
SEQ ID NO: 25 (Anti-sIL7R mAB Clone ABS-2102 Heavy Chain Amino Acid Sequence)
MGWNWIFILILSVTTGVHSEVQLQQSGPELVKPGASVKISCKASGYSFTGYYMHWVKQSPEKS
LEWIGKINPYTGGTTYNHKFKAKATLTVDKSSSTAYMQLKSLTSEDSAVYYCARSYYYAGSPP
FDYWGQGTTLTVSSAKTTAPSVYPLAPVCGDTSGSSVTLGCLVKGYFPEPVTLTWNSGSLSS
GVHTFPAVLQSDLYTLSSSVTVTSSTWPSQSITCNVAHPASSTKVDKKIEPRGPTIKPCPPCKC
PAPNLLGGPSVFIFPPKIKDVLMISLSPIVTCVVVDVSEDDPDVQISWFVNNVEVHTAQTQTHRE
DYNSTLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEKKNWVE
RNSYSCSVVHEGLHNHHTTKSFSRTPGK
SEQ ID NO: 26 (Anti-sIL7R mAB Clone ABS-2102 Light Chain DNA Sequence)
ATGGATTTTCAGGTGCAGATTTTCAGCTTCCTGCTAATCAGTGCCTCAGTCATAATGTCCAG
AGGAGAAAATGTGCTCACCCAGTCTCCAGCAATCATGTCTGCAACTCTAGGGGAGAAGGT
CACCATGAGCTGCAGGGCCAGCTCAAATGTAAAGTACATGTACTGGTACCAGCAGAAGTC
AGGTGCCTCCCCCAAACTATGGATTTATTACACATCCAACCTGGCTTCTGGAGTCCCAACT
CGCTTCAGTGGCAGTGGGTCTGGGACCTCTTATTCTCTCACACTCAGCAGCGTGGAGGCT
GAAGATGCTGCCACTTATTACTGCCAGCAGTTTACTAGTTCCCCATGGACGTTCGGTGGA
GGCACCAAGCTGGAAATCAAACGGGCTGATGCTGCACCAACTGTATCCATCTTCCCACCAT
CCAGTGAGCAGTTAACATCTGGAGGTGCCTCAGTCGTGTGCTTCTTGAACAACTTCTACCC
CAAAGACATCAATGTCAAGTGGAAGATTGATGGCAGTGAACGACAAAATGGCGTCCTGAAC
AGTTGGACTGATCAGGACAGCAAAGACAGCACCTACAGCATGAGCAGCACCCTCACGTTG
ACCAAGGACGAGTATGAACGACATAACAGCTATACCTGTGAGGCCACTCACAAGACATCAA
CTT CACCCATCGTCAAGAGCTTCAACAGGAATGAGTGTTAG
SEQ ID NO: 27 (Anti-sIL7R mAB Clone ABS-2102 Light Chain Amino Acid Sequence)
MDFQVQIFSFLLISASVIMSRGENVLTQSPAIMSATLGEKVTMSCRASSNVKYMYWYQQKSGA
SPKLWIYYTSNLASGVPTRFSGSGSGTSYSLTLSSVEAEDAATYYCQQFTSSPWTFGGGTKLE
IKRADAAPTVSIFPPSSEQLTSGGASVVCFLNNFYPKDINVKWKIDGSERQNGVLNSWTDQDSK
DSTYSMSSTLT LTKDEYERHNSYTCEATHKTSTSPIVKSFNRNEC
SEQ ID NO: 28 (Anti-sIL7R mAB Clone ABS-2022 Heavy Chain DNA Sequence)
ATGGGTTGGCTGTGGAACTTGCTATTCCTGATGGCAGCTGCCCAAAGTGCCCAAGCACAG
ATCCAGTTGGTACAGTCTGGACCTGAGCTGAAGAAGCCTGGAGAGACAGTCAAGATCTCC
TGCAAGGCTTCTGGGTATACCTTCACAACCTATGGAATGAACTGGGTGAAACAGGCTCCA
GGAAAGGGTTTAAAGTGGATGGGCTGGATAAACACCTACTCTGGAGTGCCAACATATGCT
GATGACTTCAAGGGACGGTTTGCCTTCTCTTTGGAAACCTCTGCCAGCACTGCCTATTTGC
AGATCAACAACCTCAAAAATGAGGACACGGCTACATATTTCTGTACAAGATCTGGGGGATT
ACGACCCTGGTTTGCTTACTGGGGCCAAGGGACTCTGGTCACTGTCTCTGCAGCCAAAAC
AACAGCCCCATCGGTCTATCCACTGGCCCCTGTGTGTGGAGGTACAACTGGCTCCTCGGT
GACTCTAGGATGCCTGGTCAAGGGTTATTTCCCTGAGCCAGTGACCTTGACCTGGAACTCT
GGATCCCTGTCCAGTGGTGTGCACACCTTCCCAGCTCTCCTGCAGTCTGGCCTCTACACC
CTCAGCAGCTCAGTGACTGTAACCTCGAACACCTGGCCCAGCCAGACCATCACCTGCAAT
GTGGCCCACCCGGCAAGCAGCACCAAAGTGGACAAGAAAATTGAGCCCAGAGTGCCCATA
ACACAGAACCCCTGTCCTCCACTCAAAGAGTGTCCCCCATGCGCAGCTCCAGACCTCTTG
GGTGGACCATCCGTCTTCATCTTCCCTCCAAAGATCAAGGATGTACTCATGATCTCCCTGA
GCCCCATGGTCACATGTGTGGTGGTGGATGTGAGCGAGGATGACCCAGACGTCCAGATCA
GCTGGTTTGTGAACAACGTGGAAGTACACACAGCTCAGACACAAACCCATAGAGAGGATTA
CAACAGTACTCTCCGGGTGGTCAGTGCCCTCCCCATCCAGCACCAGGACTGGATGAGTGG
CAAGGAGTTCAAATGCAAGGTCAACAACAGAGCCCTCCCATCCCCCATCGAGAAAACCATC
TCAAAACCCAGAGGGCCAGTAAGAGCTCCACAGGTATATGTCTTGCCTCCACCAGCAGAA
GAGATGACTAAGAAAGAGTTCAGTCTGACCTGCATGATCACAGGCTTCTTACCTGCCGAAA
TTGCTGTGGACTGGACCAGCAATGGGCGTACAGAGCAAAACTACAAGAACACCGCAACAG
TCCTGGACTCTGATGGTTCTTACTTCATGTACAGCAAGCTCAGAGTACAAAAGAGCACTTG
GGAAAGAGGAAGTCTTTTCGCCTGCTCAGTGGTCCACGAGGGTCTGCACAATCACCTTAC
GACTAAGACCATCTCCCGG TCTCTGGGTAAATGA
SEQ ID NO: 29 (Anti-sIL7R mAB Clone ABS-2022 Heavy Chain Amino Acid Sequence)
MGWLWNLLFLMAAAQSAQAQIQLVQSGPELKKPGETVKISCKASGYTFTTYGMNWVKQAPGK
GLKWMGWINTYSGVPTYADDFKGRFAFSLETSASTAYLQINNLKNEDTATYFCTRSGGLRPW
FAYWGQGTLVTVSAAKTTAPSVYPLAPVCGGTTGSSVTLGCLVKGYFPEPVTLTWNSGSLSS
GVHTFPALLQSGLYTLSSSVTVTSNTWPSQTITCNVAHPASSTKVDKKIEPRVPITQNPCPPLKE
CPPCAAPDLLGGPSVFIFPPKIKDVLMISLSPMVTCVVVDVSEDDPDVQISWFVNNVEVHTAQT
QTHREDYNSTLRVVSALPIQHQDWMSGKEFKCKVNNRALPSPIEKTISKPRGPVRAPQVYVLP
PPAEEMTKKEFSLTCMITGFLPAEIAVDWTSNGRTEQNYKNTATVLDSDGSYFMYSKLRVQKS
TWERGSLFACSVVHEGLHNHLTTKTISRSLGK
SEQ ID NO: 30 (Anti-sIL7R mAB Clone ABS-2022 Light Chain DNA Sequence)
ATGAGTCCTGCCCAGTTCCTGTTTCTGCTAGTGCTCTCGATTCAGGAAACCAATGGTGATG
TTGTGATGACCCAGACTCCACTGTCTTTGTCGGTTACCATTGGACAACCAGCCTCCATCTC
TTGCAAGTCAAGTCAGAGCCTCTTACATAGTAATGGAAAGACATATTTGAATTGGTTATTA
CAGAGGCCAGGCCAGTCTCCAAAGCTCCTAATCTATCTGGTGTCTAAACTGGAATCTGGC
ATCCCTGACAGGTTCAGTGGCAGTGGATCAGGGACAGATTTCACACTGAAAATCAGCAGA
GTGGAGGTTGAGGATTTGGGAGTTTATTCCTGCTTGCAACATACACATTTTCCTCATACGT
TCGGATCGGGGACCAAGCTGGAAATAAAACGGGCTGATGCTGCACCAACTGTATCCATCT
TCCCACCATCCAGTGAGCAGTTAACATCTGGAGGTGCCTCAGTCGTGTGCTTCTTGAACAA
CTTCTACCCCAAAGACATCAATGTCAAGTGGAAGATTGATGGCAGTGAACGACAAAATGGC
GTCCTGAACAGTTGGACTGATCAGGACAGCAAAGACAGCACCTACAGCATGAGCAGCACC
CTCACGTTGACCAAGGACGAGTATGAACGACATAACAGCTATACCTGTGAGGCCACTCACA
AGA CATCAACTTCACCCATCGTCAAGAGCTTCAACAGGAATGAGTGTTAG
SEQ ID NO: 31 (Anti-sIL7R mAB Clone ABS-2022 Light Chain Amino Acid Sequence)
MSPAQFLFLLVLSIQETNGDVVMTQTPLSLSVTIGQPASISCKSSQSLLHSNGKTYLNWLLQRP
GQSPKLLIYLVSKLESGIPDRFSGSGSGTDFTLKISRVEVEDLGVYSCLQHTHFPHTFGSGTKL
EIKRADAAPTVSIFPPSSEQLTSGGASVVCFLNNFYPKDINVKWKIDGSERQNGVLNSWTDQDS
KDSTYSMSS TLTLTKDEYERHNSYTCEATHKTSTSPIVKSFNRNEC
SEQ ID NO: 32 (Anti-sIL7R mAB Clone ABS-2018 Heavy Chain DNA Sequence)
ATGGAATGGATCTGGATCTTTCTCTTCATCCTGTCAGGAACTGCAGGTGTCCAATCCCAGG
TTCAGCTGCAGCAGTCTGGAGCTGAGCTGGCGAGGCCTGGGGCTTCAGTGAAGCTGTCCT
GCAAGGCTTCTGGCTACACCTTCACAAGCTATGGTATAAGCTGGGTGAAGCAGAGAACTG
GACAGGGCCTTGAGTGGTTTGGAGAGATTTATCCTAGAAGTGGTAATACTTACTACAATG
AGAAGTTCAAGGTCAAGGCCACACTGACTGCAGACAAGTCCTCCAGCACAGCGTACATGG
AGCTCCGCAGCCTGACATCTGAGGACTCTGCGGTCTATTTCTGTGCAAGATCAGGGCCAC
CTTATTACTATGCTATGGACTACTGGGGTCAAGGAACCTCAGTCACCGTCTCCTCAGCCAA
AACAACACCCCCATCAGTCTATCCACTGGCCCCTGGGTGTGGAGATACAACTGGTTCCTCT
GTGACTCTGGGATGCCTGGTCAAGGGCTACTTCCCTGAGTCAGTGACTGTGACTTGGAAC
TCTGGATCCCTGTCCAGCAGTGTGCACACCTTCCCAGCTCTCCTGCAGTCTGGACTCTAC
ACTATGAGCAGCTCAGTGACTGTCCCCTCCAGCACCTGGCCAAGTCAGACCGTCACCTGC
AGCGTTGCTCACCCAGCCAGCAGCACCACGGTGGACAAAAAACTTGAGCCCAGCGGGCC
CATTTCAACAATCAACCCCTGTCCTCCATGCAAGGAGTGTCACAAATGCCCAGCTCCTAAC
CTCGAGGGTGGACCATCCGTCTTCATCTTCCCTCCAAATATCAAGGATGTACTCATGATCT
CCCTGACACCCAAGGTCACGTGTGTGGTGGTGGATGTGAGCGAGGATGACCCAGACGTC
CGGATCAGCTGGTTTGTGAACAACGTGGAAGTACACACAGCTCAGACACAAACCCATAGA
GAGGATTACAACAGTACTATCCGGGTGGTCAGTGCCCTCCCCATCCAGCACCAGGACTGG
ATGAGTGGCAAGGAGTTCAAATGCAAGGTCAACAACAAAGACCTCCCATCACCCATCGAGA
GAACCATCTCAAAAATTAAAGGGCTAGTCAGAGCTCCACAAGTATACATCTTGCCGCCACC
AGCAGAGCAGTTGTCCAGGAAAGATGTCAGTCTCACTTGCCTGGTCGTGGGCTTCAACCC
TGGAGACATCAGTGTGGAGTGGACCAGCAATGGGCATACAGAGGAGAACTACAAGGACAC
CGCACCAGTCCTGGACTCTGACGGTTCTTACTTCATATACAGCAAGCTCGATATAAAAACA
AGCAAGTGGGAGAAAACAGATTCCTTCTCATGCAACGTGAGACACGAGGGTCTGAAAAATT
ACTACCTGAAGAAGACCATC TCCCGGTCTCCGGGTAAATGA
SEQ ID NO: 33 (Anti-sIL7R mAB Clone ABS-2018 Heavy Chain Amino Acid Sequence)
MEWIWIFLFILSGTAGVQSQVQLQQSGAELARPGASVKLSCKASGYTFTSYGISWVKQRTGQG
LEWFGEIYPRSGNTYYNEKFKVKATLTADKSSSTAYMELRSLTSEDSAVYFCARSGPPYYYAM
DYWGQGTSVTVSSAKTTPPSVYPLAPGCGDTTGSSVTLGCLVKGYFPESVTVTWNSGSLSSS
VHTFPALLQSGLYTMSSSVTVPSSTWPSQTVTCSVAHPASSTTVDKKLEPSGPISTINPCPPCK
ECHKCPAPNLEGGPSVFIFPPNIKDVLMISLTPKVTCVVVDVSEDDPDVRISWFVNNVEVHTAQ
TQTHREDYNSTIRVVSALPIQHQDWMSGKEFKCKVNNKDLPSPIERTISKIKGLVRAPQVYILPP
PAEQLSRKDVSLTCLVVGFNPGDISVEWTSNGHTEENYKDTAPVLDSDGSYFIYSKLDIKTSKW
EKTDSFSCNVRHEGLKNYYLKKTISRSPGK
SEQ ID NO: 34 (Anti-sIL7R mAB Clone ABS-2018 Light Chain DNA Sequence)
ATGAAGTTGCCTGTTAGGTTGTTGGTGCTGTTGTTCTGGATTCCTGCTTCCAGCAGTGATG
TTGTGATGACCCAAACTCCACTCTCCCTGCCTGTCAGTCTTGGAGATCAAGCTTCCATCTC
TTGCAGATCTAGTCAGAGCCTTGTACACAGCAATGGAAACACCTATTTATATTGGTACCT
GCAGAAGCCAGGCCAGTCTCCAAAGGTCCTGATCTACAGGGTTTCCAACCGATTTTCTGG
GGTCCCAGACAGGTTCAGTGGCAGTGGATCAGGGACAGATTTCACACTCAAGATCAGCAG
AGTGGAGGCTGAGGATCTGGGAGTTTATTTCTGCTTTCAAGGTACACATGTTCCGTGGAC
GTTCGGTGGAGGCACCAAGCTGGAAATCAAACGGGCTGATGCTGCACCAACTGTATCCAT
CTTCCCACCATCCAGTGAGCAGTTAACATCTGGAGGTGCCTCAGTCGTGTGCTTCTTGAAC
AACTTCTACCCCAAAGACATCAATGTCAAGTGGAAGATTGATGGCAGTGAACGACAAAATG
GCGTCCTGAACAGTTGGACTGATCAGGACAGCAAAGACAGCACCTACAGCATGAGCAGCA
CCCTCACGTTGACCAAGGACGAGTATGAACGACATAACAGCTATACCTGTGAGGCCACTCA
CAAGACATCAACTTCACCCATCGTCAAGAGCTTCAACAGGAATGAGTGTTAG
SEQ ID NO: 35 (Anti-sIL7R mAB Clone ABS-2018 Light Chain Amino Acid Sequence)
MKLPVRLLVLLFWIPASSSDVVMTQTPLSLPVSLGDQASISCRSSQSLVHSNGNTYLYWYLQK
PGQSPKVLIYRVSNRFSGVPDRFSGSGSGTDFTLKISRVEAEDLGVYFCFQGTHVPWTFGGGT
KLEIKRADAAPTVSIFPPSSEQLTSGGASVVCFLNNFYPKDINVKWKIDGSERQNGVLNSWTDQ
DSKDSTYSMSS TLTLTKDEYERHNSYTCEATHKTSTSPIVKSFNRNEC
SEQ ID NO: 36 (Anti-sIL7R mAB Clone ABS-2028 Heavy Chain DNA Sequence)
ATGGGTTGGCTGTGGAACTTGCTATTCCTGATGGCAGCTGCCCAAAGTGCCCAAGCACAA
TCCAGTTGGTACAGTCTGGACCTGAGCTGAAGAAGCCTGGAGAGACAGTCAAGATCTCCT
GCAAGGCTTCTGGGTATACCTTCACAACCTATGGAATGAACTGGGTGAAACAGGCTCCAG
GAAAGGGTTTAAAGTGGATGGGCTGGATAAACACCTACTCTGGAGTGCCAACATATGCTG
ATGACTTCAAGGGACGGTTTGCCTTCTCTTTGGAAACCTCTGCCAGCACTGCCTATTTGCA
GATCAACAACCTCAAAAATGAGGACACGGCTACATATTTCTGTACAAGATCTGGGGGATTA
CGACCCTGGTTTGCTTACTGGGGCCAAGGGACTCTGGTCACTGTCTCTGCAGCCAAAACA
ACAGCCCCATCGGTCTATCCACTGGCCCCTGTGTGTGGAGGTACAACTGGCTCCTCGGTG
ACTCTAGGATGCCTGGTCAAGGGTTATTTCCCTGAGCCAGTGACCTTGACCTGGAACTCTG
GATCCCTGTCCAGTGGTGTGCACACCTTCCCAGCTCTCCTGCAGTCTGGCCTCTACACCCT
CAGCAGCTCAGTGACTGTAACCTCGAACACCTGGCCCAGCCAGACCATCACCTGCAATGT
GGCCCACCCGGCAAGCAGCACCAAAGTGGACAAGAAAATTGAGCCCAGAGTGCCCATAAC
ACAGAACCCCTGTCCTCCACTCAAAGAGTGTCCCCCATGCGCAGCTCCAGACCTCTTGGG
TGGACCATCCGTCTTCATCTTCCCTCCAAAGATCAAGGATGTACTCATGATCTCCCTGAGC
CCCATGGTCACATGTGTGGTGGTGGATGTGAGCGAGGATGACCCAGACGTCCAGATCAGC
TGGTTTGTGAACAACGTGGAAGTACACACAGCTCAGACACAAACCCATAGAGAGGATTACA
ACAGTACTCTCCGGGTGGTCAGTGCCCTCCCCATCCAGCACCAGGACTGGATGAGTGGCA
AGGAGTTCAAATGCAAGGTCAACAACAGAGCCCTCCCATCCCCCATCGAGAAAACCATCTC
AAAACCCAGAGGGCCAGTAAGAGCTCCACAGGTATATGTCTTGCCTCCACCAGCAGAAGA
GATGACTAAGAAAGAGTTCAGTCTGACCTGCATGATCACAGGCTTCTTACCTGCCGAAATT
GCTGTGGACTGGACCAGCAATGGGCGTACAGAGCAAAACTACAAGAACACCGCAACAGTC
CTGGACTCTGATGGTTCTTACTTCATGTACAGCAAGCTCAGAGTACAAAAGAGCACTTGGG
AAAGAGGAAGTCTTTTCGCCTGCTCAGTGGTCCACGAGGGTCTGCACAATCACCTTACGAC
TAAGACCATCTCCCGG TCTCTGGGTAAATGA
SEQ ID NO: 37 (Anti-sIL7R mAB Clone ABS-2028 Heavy Chain Amino Acid Sequence)
MGWLWNLLFLMAAAQSAQAQIQLVQSGPELKKPGETVKISCKASGYTFTTYGMNWVKQAPGK
GLKWMGWINTYSGVPTYADDFKGRFAFSLETSASTAYLQINNLKNEDTATYFCTRSGGLRPW
FAYWGQGTLVTVSAAKTTAPSVYPLAPVCGGTTGSSVTLGCLVKGYFPEPVTLTWNSGSLSS
GVHTFPALLQSGLYTLSSSVTVTSNTWPSQTITCNVAHPASSTKVDKKIEPRVPITQNPCPPLKE
CPPCAAPDLLGGPSVFIFPPKIKDVLMISLSPMVTCVVVDVSEDDPDVQISWFVNNVEVHTAQT
QTHREDYNSTLRVVSALPIQHQDWMSGKEFKCKVNNRALPSPIEKTISKPRGPVRAPQVYVLP
PPAEEMTKKEFSLTCMITGFLPAEIAVDWTSNGRTEQNYKNTATVLDSDGSYFMYSKLRVQKS
TWERGSLFACSVVHEGLHNHLTTKTISRSLGK
SEQ ID NO: 38 (Anti-sIL7R mAB Clone ABS-2028 Light Chain DNA Sequence)
ATGAGTCCTGCCCAGTTCCTGTTTCTGCTAGTGCTCTCGATTCAGGAAACCAATGGTGATG
TTGTGATGACCCAGACTCCACTGTCTTTGTCGGTTACCATTGGACAACCAGCCTCCATCTC
TTGCAAGTCAAGTCAGAGCCTCTTACATAGTAATGGAAAGACATATTTGAATTGGTTATTA
CAGAGGCCAGGCCAGTCTCCAAAGCTCCTAATCTATCTGGTGTCTAAACTGGAATCTGGC
ATCCCTGACAGGTTCAGTGGCAGTGGATCAGGGACAGATTTCACACTGAAAATCAGCAGA
GTGGAGGTTGAGGATTTGGGAGTTTATTCCTGCTTGCAACATACACATTTTCCTCATACGT
TCGGATCGGGGACCAAGCTGGAAATAAAACGGGCTGATGCTGCACCAACTGTATCCATCT
TCCCACCATCCAGTGAGCAGTTAACATCTGGAGGTGCCTCAGTCGTGTGCTTCTTGAACAA
CTTCTACCCCAAAGACATCAATGTCAAGTGGAAGATTGATGGCAGTGAACGACAAAATGGC
GTCCTGAACAGTTGGACTGATCAGGACAGCAAAGACAGCACCTACAGCATGAGCAGCACC
CTCACGTTGACCAAGGACGAGTATGAACGACATAACAGCTATACCTGTGAGGCCACTCACA
AGA CATCAACTTCACCCATCGTCAAGAGCTTCAACAGGAATGAGTGTTAG
SEQ ID NO: 39 (Anti-sIL7R mAB Clone ABS-2028 Light Chain Amino Acid Sequence)
MSPAQFLFLLVLSIQETNGDVVMTQTPLSLSVTIGQPASISCKSSQSLLHSNGKTYLNWLLQRP
GQSPKLLIYLVSKLESGIPDRFSGSGSGTDFTLKISRVEVEDLGVYSCLQHTHFPHTFGSGTKL
EIKRADAAPTVSIFPPSSEQLTSGGASVVCFLNNFYPKDINVKWKIDGSERQNGVLNSWTDQDS
KDSTYSMSS TLTLTKDEYERHNSYTCEATHKTSTSPIVKSFNRNEC
SEQ ID NO: 40 (Anti-sIL7R mAB Clone ABS-2045 Heavy Chain DNA Sequence)
ATGAACTTTGTGCTCAGCTTGATTTTCCTTGCCCTCATTTTAAAAGGTGTCCAGTGTGAAGT
GCAGCTGGTGGAGTCTGGGGGAGGCTTAGTGAAGCCTGGAGGGTCCCTAAAACTCTCCT
GTGCTGCCTCTGGATTCACTTTCAGTAGCTATGCCATGTCTTGGGTTCGCCAGACTCCGGA
GAAGAGGCTGGAGTGGGTCGCAAGCATTAGTAGTGGTGGTAGTTACACCTACTATCCAG
ACAGTGTGAAGGGTCGATTCACCATCTCCAGAGACAATGCCAAGAACACCCTGTACCTGC
AAATGAGCAGTCTGAGGTCTGAGGACACGGCCATGTATTACTGTGCAAGACTGGGCACCG
TTTACTATGCTATGGACTACTGGGGTCAAGGAACCTCAGTCACCGTCTCCTCAGCCAAAAC
AACACCCCCATCAGTCTATCCACTGGCCCCTGGGTGTGGAGATACAACTGGTTCCTCTGTG
ACTCTGGGATGCCTGGTCAAGGGCTACTTCCCTGAGTCAGTGACTGTGACTTGGAACTCT
GGATCCCTGTCCAGCAGTGTGCACACCTTCCCAGCTCTCCTGCAGTCTGGACTCTACACTA
TGAGCAGCTCAGTGACTGTCCCCTCCAGCACCTGGCCAAGTCAGACCGTCACCTGCAGCG
TTGCTCACCCAGCCAGCAGCACCACGGTGGACAAAAAACTTGAGCCCAGCGGGCCCATTT
CAACAATCAACCCCTGTCCTCCATGCAAGGAGTGTCACAAATGCCCAGCTCCTAACCTCGA
GGGTGGACCATCCGTCTTCATCTTCCCTCCAAATATCAAGGATGTACTCATGATCTCCCTG
ACACCCAAGGTCACGTGTGTGGTGGTGGATGTGAGCGAGGATGACCCAGACGTCCGGAT
CAGCTGGTTTGTGAACAACGTGGAAGTACACACAGCTCAGACACAAACCCATAGAGAGGA
TTACAACAGTACTATCCGGGTGGTCAGTGCCCTCCCCATCCAGCACCAGGACTGGATGAG
TGGCAAGGAGTTCAAATGCAAGGTCAACAACAAAGACCTCCCATCACCCATCGAGAGAAC
CATCTCAAAAATTAAAGGGCTAGTCAGAGCTCCACAAGTATACATCTTGCCGCCACCAGCA
GAGCAGTTGTCCAGGAAAGATGTCAGTCTCACTTGCCTGGTCGTGGGCTTCAACCCTGGA
GACATCAGTGTGGAGTGGACCAGCAATGGGCATACAGAGGAGAACTACAAGGACACCGCA
CCAGTCCTGGACTCTGACGGTTCTTACTTCATATACAGCAAGCTCGATATAAAAACAAGCAA
GTGGGAGAAAACAGATTCCTTCTCATGCAACGTGAGACACGAGGGTCTGAAAAATTACTAC
CTGAAGAAGACCATCTCC CGGTCTCCGGGTAAATGA
SEQ ID NO: 41 (Anti-sIL7R mAB Clone ABS-2045 Heavy Chain Amino Acid Sequence)
MNFVLSLIFLALILKGVQCEVQLVESGGGLVKPGGSLKLSCAASGFTFSSYAMSWVRQTPEKRL
EWVASISSGGSYTYYPDSVKGRFTISRDNAKNTLYLQMSSLRSEDTAMYYCARLGTVYYAMDY
WGQGTSVTVSSAKTTPPSVYPLAPGCGDTTGSSVTLGCLVKGYFPESVTVTWNSGSLSSSVH
TFPALLQSGLYTMSSSVTVPSSTWPSQTVTCSVAHPASSTTVDKKLEPSGPISTINPCPPCKEC
HKCPAPNLEGGPSVFIFPPNIKDVLMISLTPKVTCVVVDVSEDDPDVRISWFVNNVEVHTAQTQ
THREDYNSTIRVVSALPIQHQDWMSGKEFKCKVNNKDLPSPIERTISKIKGLVRAPQVYILPPPA
EQLSRKDVSLTCLVVGFNPGDISVEWTSNGHTEENYKDTAPVLDSDGSYFIYSKLDIKTSKWEK
TDSFSCNVRHEGLKNYYLKKTISRSPGK
SEQ ID NO: 42 (Anti-sIL7R mAB Clone ABS-2045 Light Chain DNA Sequence)
ATGGATTTTCAGGTGCAGATTTTCAGCTTCCTGCTAATCAGTGCCTCAGTCATAATGTCCAG
AGGACAAATTGTTCTCACCCAGTCTCCAGCAATCATGTCTGCATCTCTAGGGGAGGAGATC
ACCCTAACCTGCAGTGCCAGCTCGAGTGTAAGTTACATGCACTGGTACCAGCAGAAGTCA
GGCACTTCTCCCAAACTCTTGATTTATAGCACATCCAACCTGGCTTCTGGAGTCCCTTCTC
GCTTCAGTGGCAGTGGGTCTGGGACCTTTTATTCTCTCACAATCAGCAGTGTGGAGGCTGA
AGATGCTGCCGATTATTACTGCCATCAGTGGAGTAGTTATCGGACGTTCGGTGGAGGCAC
CAAGCTGGAAATCAAACGGGCTGATGCTGCACCAACTGTATCCATCTTCCCACCATCCAGT
GAGCAGTTAACATCTGGAGGTGCCTCAGTCGTGTGCTTCTTGAACAACTTCTACCCCAAAG
ACATCAATGTCAAGTGGAAGATTGATGGCAGTGAACGACAAAATGGCGTCCTGAACAGTTG
GACTGATCAGGACAGCAAAGACAGCACCTACAGCATGAGCAGCACCCTCACGTTGACCAA
GGACGAGTATGAACGACATAACAGCTATACCTGTGAGGCCACTCACAAGACATCAACTTCA
CCCATTGTCAAGAGCTTCAACAGGAATGAGTGTTAG
SEQ ID NO: 43 (Anti-sIL7R mAB Clone ABS-2045 Light Chain Amino Acid Sequence)
MDFQVQIFSFLLISASVIMSRGQIVLTQSPAIMSASLGEEITLTCSASSSVSYMHWYQQKSGTSP
KLLIYSTSNLASGVPSRFSGSGSGTFYSLTISSVEAEDAADYYCHQWSSYRTFGGGTKLEIKRA
DAAPTVSIFPPSSEQLTSGGASVVCFLNNFYPKDINVKWKIDGSERQNGVLNSWTDQDSKDST
YSMSSTLTL TKDEYERHNSYTCEATHKTSTSPIVKSFNRNEC

TABLE 2
Heavy (HCDR) and Light Change (LCDR) Amino Acid Sequences

| Seq ID | sequence | name |
|---|---|---|
| 44 | TYGMS | clone 2024, |
| | | HCDR1 |
| 45 | CINTYSGVPTYPDDFKG | clone 2024, |
| | | HCDR2 |
| 46 | EKNYYGARYYFDF | clone 2024, |
| | | HCDR3 |
| 47 | RSSQSIVHSYRYTYLE | clone 2024, |
| | | LCDR1 |
| 48 | GVSNRFS | clone 2024, |
| | | LCDR2 |
| 49 | FQGTHVPLT | clone 2024, |
| | | LCDR3 |
| 50 | TYGMGVG | clone 2096, |
| | | HCDR1 |
| 51 | NIWWNDYKYYNSALKS | clone 2096, |
| | | HCDR2 |
| 52 | VGNYDGGFH | clone 2096, |
| | | HCDR3 |
| 53 | RSSQTLVHSNGNTYLH | clone 2096, |
| | | LCDR1 |
| 54 | KVSNRFS | clone 2096, |
| | | LCDR2 |
| 55 | SQSTHVPWT | clone 2096, |
| | | LCDR3 |
| 56 | TYGMN | clone 2005, |
| | | HCDR1 |
| 57 | WINTYSGVPTYVDDFKG | clone 2005, |
| | | HCDR2 |
| 58 | WGTPRYFFDY | clone 2005, |
| | | HCDR3 |
| 59 | RSSQSIVHSNRYTYLE | clone 2005, |
| | | LCDR1 |
| 60 | GVSNRFS | clone 2005, |
| | | LCDR2 |
| 61 | FQGTHVPWT | clone 2005, |
| | | LCDR3 |
| 62 | TYGMN | clone 2008, |
| | | HCDR1 |
| 63 | WINTYSGVPTYADDFKG | clone 2008, |
| | | HCDR2 |
| 64 | SGGLRPWFAY | clone 2008, |
| | | HCDR3 |
| 65 | KSSQSLLHSNGKTYLN | clone 2008, |
| | | LCDR1 |
| 66 | LVSKLES | clone 2008, |
| | | LCDR2 |
| 67 | LQHTHFPHT | clone 2008, |
| | | LCDR3 |
| 68 | RYAMS | clone 2014, |
| | | HCDR1 |
| 69 | TINDGGSYTYYPDSVKG | clone 2014, |
| | | HCDR2 |
| 70 | EGYYGSFDY | clone 2014 |
| | | HCDR3 |
| 72 | KSSQSLLYTNGKTYLN | clone 2014, |
| | | LCDR1 |
| 72 | LVSKLDS | clone 2014, |
| | | LCDR2 |
| 73 | LQSTRFPQT | clone 2014, |
| | | LCDR3 |
| 74 | GYYMH | clone 2102, |
| | | HCDR1 |
| 75 | KINPYTGGTTYNHKFKA | clone 2102, |
| | | HCDR2 |
| 76 | SYYYAGSPPFDY | clone 2102, |
| | | HCDR3 |
| 77 | RASSNVKYMY | clone 2102, |
| | | LCDR1 |
| 78 | YTSNLAS | clone 2102, |
| | | LCDR2 |
| 79 | QQFTSSPWT | clone 2102, |
| | | LCDR3 |
| 80 | TYGMN | clone 2022, |
| | | HCDR1 |
| 81 | WINTYSGVPTYADDFKG | clone 2022, |
| | | HCDR2 |
| 82 | SGGLRPWFAY | clone 2022, |
| | | HCDR3 |
| 83 | KSSQSLLHSNGKTYLN | clone 2022, |
| | | LCDR1 |
| 84 | LVSKLES | clone 2022, |
| | | LCDR2 |
| 85 | LQHTHFPHT | clone 2022, |
| | | LCDR3 |
| 86 | SYGIS | clone 2018, |
| | | HCDR1 |
| 87 | EIYPRSGNTYYNEKFKV | clone 2018, |
| | | HCDR2 |
| 88 | SGPPYYYAMDY | clone 2018, |
| | | HCDR3 |
| 89 | RSSQSLVHSNGNTYLY | clone 2018, |
| | | LCDR1 |
| 90 | RVSNRFS | clone 2018, |
| | | LCDR2 |
| 91 | FQGTHVPWT | clone 2018, |
| | | LCDR3 |
| 92 | TYGMN | clone 2028, |
| | | HCDR1 |
| 93 | WINTYSGVPTYADDFKG | clone 2028, |
| | | HCDR2 |
| 94 | SGGLRPWFAY | clone 2028, |
| | | HCDR3 |
| 95 | KSSQSLLHSNGKTYLN | clone 2028 |
| | | LCDR1 |
| 96 | LVSKLES | clone 2028, |
| | | LCDR2 |
| 97 | LQHTHFPHT | clone 2028, |
| | | LCDR3 |
| 98 | SASSSVSYMH | clone 2045, |
| | | LCDR1 |
| 99 | STSNLAS | clone 2045, |
| | | LCDR2 |
| 100 | HQWSSYRT | clone 2045 |
| | | LCDR3 |

1.-40. (canceled)

41. A pharmaceutical composition comprising anti-sIL7R antibodies that bind to and neutralize sIL7R (soluble isoform of the interleukin 7 receptor) or reduce sIL7R from circulation without inhibiting the alpha chain of the interleukin 7 receptor (mIL7R).

42. A method of treating an autoimmune disease comprising administering a therapeutic amount of the pharmaceutical composition of claim 41.

43. The method of claim 42, wherein the autoimmune disease is one or more of multiple sclerosis, type 1 diabetes, rheumatoid arthritis, systemic lupus erythematosus, Sjøgren's syndrome, Crohn's disease, ulcerative colitis, celiac disease, psoriasis, atopic dermatitis, ankylosing spondylitis, primary biliary cirrhosis, hepatitis, autoimmune encephalomyelitis, autoimmune hepatitis, nephritis, lupus nephritis, Graves' disease, myasthenia gravis, myelin oligodendrocyte glycoprotein antibody disorder, psoriatic arthritis, scleroderma, alopecia areata, Addison's disease, pernicious anemia, or another condition with elevated sIL7R.

44. The method of claim 42, wherein the anti-sIL7R antibodies are administered intravenously, subcutaneously, intradermally, topically, intranasally, intraperitoneally, intraocularly, orally, rectally, intrathecally, intraventricularly, vaginally, intramuscularly or directly into the central nervous system (CNS).

45. The pharmaceutical composition of claim 41, wherein the anti-sIL7R antibodies target one or more regions of sIL7R that are different from mIL7R.

46. The pharmaceutical composition of claim 41, wherein the anti-sIL7R antibodies further comprise one or more conservative amino acid substitutions.

47. The pharmaceutical composition of claim 41, wherein the anti-sIL7R antibodies are antibody fragments, binding proteins, fusion proteins or synthetic proteins.

48. The pharmaceutical composition of claim 41, wherein the pharmaceutical composition is formulated for sustained release or extended release.

49. The pharmaceutical composition of claim 41, wherein the anti-sIL7R antibody is comprised of a heavy chain selected from SEQ ID NO: 5, 9, 13, 17, 21, 25, 29, 33, 37 and 41; and a light chain selected from SEQ ID NO: 7, 11, 15, 19, 23, 27, 31, 35, 39 and 43.

50. An antibody which binds to sIL7R, the antibody selected from:
(a) an antibody comprising a heavy chain containing at least the amino acid sequence as set forth in SEQ ID NO: 5, and light chain containing at least the amino acid sequence as set forth in SEQ ID NO: 7;
(b) an antibody comprising a heavy chain containing at least the amino acid sequence as set forth in SEQ ID NO: 9, and light chain containing at least the amino acid sequence as set forth in SEQ ID NO: 11;
(c) an antibody comprising a heavy chain containing at least the amino acid sequence as set forth in SEQ ID NO: 13, and light chain containing at least the amino acid sequence as set forth in SEQ ID NO: 15;
(d) an antibody comprising a heavy chain containing at least the amino acid sequence as set forth in SEQ ID NO: 17, and light chain containing at least the amino acid sequence as set forth in SEQ ID NO: 19;
(e) an antibody comprising a heavy chain containing at least the amino acid sequence as set forth in SEQ ID NO: 21, and light chain containing at least the amino acid sequence as set forth in SEQ ID NO: 23;
(f) an antibody comprising a heavy chain containing at least the amino acid sequence as set forth in SEQ ID NO: 25, and light chain containing at least the amino acid sequence as set forth in SEQ ID NO: 27;
(g) an antibody comprising a heavy chain containing at least the amino acid sequence as set forth in SEQ ID NO: 29, and light chain containing at least the amino acid sequence as set forth in SEQ ID NO: 31;
(h) an antibody comprising a heavy chain containing at least the amino acid sequence as set forth in SEQ ID NO: 33, and light chain containing at least the amino acid sequence as set forth in SEQ ID NO: 35;
(i) an antibody comprising a heavy chain containing at least the amino acid sequence as set forth in SEQ ID NO: 37, and light chain containing at least the amino acid sequence as set forth in SEQ ID NO: 39; and
(j) an antibody comprising a heavy chain containing at least the amino acid sequence as set forth in SEQ ID NO: 41, and light chain containing at least the amino acid sequence as set forth in SEQ ID NO: 43.

51. The antibody of claim 50, wherein the antibody does not bind to mIL7R.

52. The antibody of claim 50, wherein the antibody binds to one of the sIL7R-specific C-terminal tail (encoded by exon 7 in transcripts that skip exon 6), and/or the region of sIL7R encoded by the junction between exons 5 and 7 in transcripts that skip exon 6.

53. A method of treating an ailment, the method comprising administering a composition comprising the antibody of claim 50.

54. The method of claim 53, wherein the ailment is an autoimmune disease.

55. The method of claim 54, wherein the autoimmune disease is one or more of multiple sclerosis, type 1 diabetes, rheumatoid arthritis, systemic lupus erythematosus, Sjøgren's syndrome, Crohn's disease, ulcerative colitis, celiac disease, psoriasis, atopic dermatitis, ankylosing spondylitis, primary biliary cirrhosis, hepatitis, autoimmune encephalomyelitis, autoimmune hepatitis, nephritis, lupus nephritis, Graves' disease, myasthenia gravis, myelin oligodendrocyte glycoprotein antibody disorder, psoriatic arthritis, scleroderma, alopecia areata, Addison's disease, pernicious anemia, or another condition with elevated sIL7R.

56. The method of claim 53, wherein the method does not suppress the immune system below normal activity.

57. The method of claim 53, wherein the method neutralizes sIL7R and/or reduces levels of circulating sIL7R without inhibiting mIL7R.

58. The method of claim 53, wherein the composition is administered with a second therapeutic agent, the second therapeutic agent selected from an immunomodulatory agent or a biologic.

59. A method of treating an autoimmune disease in a subject without suppressing the immune system below normal activity, the method comprising neutralizing and/or reducing the amount of sIL7R in the subject.

60. The method of claim 59, wherein the method comprises administering an anti-sIL7R antibody or anti-sIL7R antagonist to the subject.

61. The method of claim 59, wherein the autoimmune disease is one or more of multiple sclerosis, type 1 diabetes, rheumatoid arthritis, systemic lupus erythematosus, Sjøgren's syndrome, Crohn's disease, ulcerative colitis, celiac disease, psoriasis, atopic dermatitis, ankylosing spondylitis, primary biliary cirrhosis, hepatitis, autoimmune encephalomyelitis, autoimmune hepatitis, nephritis, lupus nephritis, Graves' disease, myasthenia gravis, myelin oligodendrocyte glycoprotein antibody disorder, psoriatic arthritis, scleroderma, alopecia areata, Addison's disease, pernicious anemia, or another condition with elevated sIL7R.

62. The method of claim 59, wherein the method comprises administering a therapeutic amount of an antibody selected from:
(a) an antibody comprising a heavy chain containing at least the amino acid sequence as set forth in SEQ ID NO: 5, and light chain containing at least the amino acid sequence as set forth in SEQ ID NO: 7;
(b) an antibody comprising a heavy chain containing at least the amino acid sequence as set forth in SEQ ID NO: 9, and light chain containing at least the amino acid sequence as set forth in SEQ ID NO: 11;
(c) an antibody comprising a heavy chain containing at least the amino acid sequence as set forth in SEQ ID NO: 13, and light chain containing at least the amino acid sequence as set forth in SEQ ID NO: 15;
(d) an antibody comprising a heavy chain containing at least the amino acid sequence as set forth in SEQ ID NO: 17, and light chain containing at least the amino acid sequence as set forth in SEQ ID NO: 19;
(e) an antibody comprising a heavy chain containing at least the amino acid sequence as set forth in SEQ ID NO: 21, and light chain containing at least the amino acid sequence as set forth in SEQ ID NO: 23;
(f) an antibody comprising a heavy chain containing at least the amino acid sequence as set forth in SEQ ID NO: 25, and light chain containing at least the amino acid sequence as set forth in SEQ ID NO: 27;
(g) an antibody comprising a heavy chain containing at least the amino acid sequence as set forth in SEQ ID NO: 29, and light chain containing at least the amino acid sequence as set forth in SEQ ID NO: 31;
(h) an antibody comprising a heavy chain containing at least the amino acid sequence as set forth in SEQ ID NO: 33, and light chain containing at least the amino acid sequence as set forth in SEQ ID NO: 35;
(i) an antibody comprising a heavy chain containing at least the amino acid sequence as set forth in SEQ ID NO: 37, and light chain containing at least the amino acid sequence as set forth in SEQ ID NO: 39; and
(j) an antibody comprising a heavy chain containing at least the amino acid sequence as set forth in SEQ ID NO: 41, and light chain containing at least the amino acid sequence as set forth in SEQ ID NO: 43.


SEQUENCE LISTING (100 sequences)

SEQ ID NO: 1 - protein, 26 aa, synthetic construct
  REGION 1..26: note = sIL7R C-terminal tail
LSLSYGPVSPIIRRLWNIFVRNQEKI

SEQ ID NO: 2 - protein, 188 aa, synthetic construct
  REGION 1..188: note = sIL7R binding region binding IL7
FSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFR

SEQ ID NO: 3 - protein, 54 aa, synthetic construct
  REGION 1..54: note = sIL7R at junction between IL7 binding region and C-terminal tail
PDHYFKGFWSEWSPSYYFRTPEINNSSGLSLSYGPVSPIIRRLWNIFVRNQEKI

SEQ ID NO: 4 - DNA, 1434 bp, synthetic construct
  misc_feature 1..1434: note = portion of human (Homo sapien)
  source 1..1434: mol_type = other DNA
atgggttggctgtggaacttgctattcctgatggcagctgcccaaagtgcccaagcacagatccagttggtacagtctggacctgagctgaagaagcctggagagacagtcaggatctcctgcaaggcttctgggtataccttcacaacctatggaatgagctgggtgaaacaggctccaggaaagggtttaaagtggatgggctgtataaacacctactctggagtgccaacatatcctgatgacttcaagggacggtttgccttctctttggaaacctctgccagcactgcctatttgcagatcaacaacctcaaaaatgaggacacggctacatatttctgtgcaagagagaagaattattacggtgctaggtactactttgacttctggggccaaggcaccactctcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 5 - protein, 477 aa, synthetic construct
  REGION 1..477: note = Heavy chain amino acid sequence
MGWLWNLLFLMAAAQSAQAQIQLVQSGPELKKPGETVRISCKASGYTFTTYGMSWVKQAPGKGLKWMGCINTYSGVPTYPDDFKGRFAFSLETSASTAYLQINNLKNEDTATYFCAREKNYYGARYYFDFWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 6 - DNA, 717 bp, synthetic construct
  misc_feature 1..717: note = portion of human
  source 1..717: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagcattgtacacagttatagatacacctatttagaatggtacctgcagaaaccaggccagtcactaaagctcctgatatatggggtttccaaccgattttctggggtcccagacaggttcagtggcagtggatcagggacagatttcacactcaagatcagcagagtggaggctgaggatatgggagtttattactgctttcaaggtacacatgttccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 7 - protein, 238 aa, synthetic construct
  REGION 1..238: note = Light chain amino acid sequence
MKLPVRLLVLMFWIPASSSDVVMTQTPLSLPVSLGDQASISCRSSQSIVHSYRYTYLEWYLQKPGQSLKLLIYGVSNRFSGVPDRFSGSGSGTDFTLKISRVEAEDMGVYYCFQGTHVPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 8 - DNA, 1392 bp, synthetic construct
  misc_feature 1..1392: note = Anti-sIL7R mAB Clone ABS-2096 Heavy Chain DNA Sequence
  source 1..1392: mol_type = other DNA
atgggcaggcttacttcttcattcctgctactgattgtccctgcatatgtcctgtcccaggtcactctgaaagagtctggccctgggatattgcagccctcccagaccctcagtctgacttgttctttctctgggttttcactgagcacttatggtatgggtgtaggttggattcgtcagccttcagggaagggtctggagtggctggccaacatttggtggaatgattataaatactataactcagccctgaagagccggctcacaatctccaaggatccccccaacaaccaggtattcctcaagatctccagtgtggacactgcagatactgccacatactactgtgctcaagtaggaaactacgatggtggcttccactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcatctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 9 - protein, 463 aa, synthetic construct
  REGION 1..463: note = Heavy chain amino acid sequence
MGRLTSSFLLLIVPAYVLSQVTLKESGPGILQPSQTLSLTCSFSGFSLSTYGMGVGWIRQPSGKGLEWLANIWWNDYKYYNSALKSRLTISKDPPNNQVFLKISSVDTADTATYYCAQVGNYDGGFHYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 10 - DNA, 717 bp, synthetic construct
  misc_feature 1..717: note = Anti-sIL7R mAB Clone ABS-2005 Heavy Chain DNA Sequence
  source 1..717: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagacccttgtacacagtaatggaaacacctatttacattggtacctgcagaggccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 11 - protein, 238 aa, synthetic construct
  REGION 1..238: note = Light chain amino acid sequence
MKLPVRLLVLMFWIPASSSDVVMTQTPLSLPVSLGDQASISCRSSQTLVHSNGNTYLHWYLQRPGQSPKLLIYKVSNRFSGVPDRFSGSGSGTDFTLKISRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - DNA, 1425 bp, synthetic construct
  misc_feature 1..1425: note = Anti-sIL7R mAB Clone ABS-2005 Heavy Chain DNA Sequence
  source 1..1425: mol_type = other DNA
atgggttggctgtggaacttgctattcctgatggcagctgcccaaagtgcccaagcacagatccagttggtacagtctggacctaaactgaagaagcctggagagacagtcaagatctcctgcagggcttctgggtatcccttcacaacctatggaatgaactgggtgaaacaggctccaggaaagggtttaaagtggatgggttggataaatacctactctggagtgccaacatatgttgatgacttcaagggacggtttgccttctctttggaaacctctgccaccactgcccatttgcagatcaacaacctcaaaaatgaggacacggctacatatttctgtgcaagatggggtactcccaggtatttctttgactattggggccaaggcaccactctcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 13 - protein, 474 aa, synthetic construct
  REGION 1..474: note = Heavy chain amino acid sequence
MGWLWNLLFLMAAAQSAQAQIQLVQSGPKLKKPGETVKISCRASGYPFTTYGMNWVKQAPGKGLKWMGWINTYSGVPTYVDDFKGRFAFSLETSATTAHLQINNLKNEDTATYFCARWGTPRYFFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 14 - DNA, 717 bp, synthetic construct
  misc_feature 1..717: note = Anti-sIL7R mAB Clone ABS-2005 Light Chain DNA Sequence
  source 1..717: mol_type = other DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagcattgtacacagtaatagatacacctatttagaatggtacctgcagaaaccaggccagtcgctaaagctcctgatatatggggtttccaaccgattttctggggtcccagacaggttcagtggcagtggatcagggacagatttcacactcaagatcagcagagtggaggctgaggatatgggagtttattactgctttcaaggtacacatgttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 15 - protein, 238 aa, synthetic construct
  REGION 1..238: note = Light chain amino acid sequence
MKLPVRLLVLMFWIPASSSDVVMTQTPLSLPVSLGDQASISCRSSQSIVHSNRYTYLEWYLQKPGQSLKLLIYGVSNRFSGVPDRFSGSGSGTDFTLKISRVEAEDMGVYYCFQGTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - DNA, 1425 bp, synthetic construct
  misc_feature 1..1425: note = Anti-sIL7R mAB Clone ABS-2008 Heavy Chain DNA Sequence
  source 1..1425: mol_type = other DNA
atgggttggctgtggaacttgctattcctgatggcagctgcccaaagtgcccaagcacagatccagttggtacagtctggacctgagctgaagaagcctggagagacagtcaagatctcctgcaaggcctctgggtataccttcacaacctatggaatgaactgggtgcaacaggctccaggaaagggtttaaagtggatgggctggataaacacctactctggagtgccaacatatgctgatgacttcaagggacggtttgccttctctttggaaacctctgccagcactgcctatttgcagatcaacaacctcaaaaatgaggacacggccaaatatttctgtacaagatctgggggattacgaccctggtttgcttactggggccaagggactctggtcactgtctctgcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 17 - protein, 474 aa, synthetic construct
  REGION 1..474: note = Heavy chain amino acid sequence
MGWLWNLLFLMAAAQSAQAQIQLVQSGPELKKPGETVKISCKASGYTFTTYGMNWVQQAPGKGLKWMGWINTYSGVPTYADDFKGRFAFSLETSASTAYLQINNLKNEDTAKYFCTRSGGLRPWFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 18 - DNA, 717 bp, synthetic construct
  misc_feature 1..717: note = Anti-sIL7R mAB Clone ABS-2008 Light Chain DNA Sequence
  source 1..717: mol_type = other DNA
atgagtcctgcccagttcctgtttctgctagtgctctcgattcaggaaaccaatggtgatgttgtgatgacccagactccactgtctttgtcggttaccattggacaaccagcctccatctcttgcaagtcaagtcagagcctcttacatagtaatggaaagacatatttgaattggttattacagaggccaggccagtctccaaagctcctaatctatctggtgtctaaactggaatctggcatccctgacaggttcagtggcagtggatcagggacagatttcacactgaaaatcagcagagtggaggttgaggatttgggagtttattactgcttgcaacatacacattttcctcatacgttcggatcggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 19 - protein, 238 aa, synthetic construct
  REGION 1..238: note = Light chain amino acid sequence
MSPAQFLFLLVLSIQETNGDVVMTQTPLSLSVTIGQPASISCKSSQSLLHSNGKTYLNWLLQRPGQSPKLLIYLVSKLESGIPDRFSGSGSGTDFTLKISRVEVEDLGVYYCLQHTHFPHTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - DNA, 1422 bp, synthetic construct
  misc_feature 1..1422: note = Anti-sIL7R mAB Clone ABS-2014 Heavy Chain DNA Sequence
  source 1..1422: mol_type = other DNA
atgaactttgtgctcagcttgattttccttgccctcattttaaaaggtgtccagtgtgaagtgcagctggtggagtctgggggaggctcagtgaagcctggagggtccctgaaactctcctgtgcagcctctggattcactttcagtcgctatgccatgtcttgggttcgccagactccggagaagaggctggagtgggtcgcaaccattaatgatggtggtagttacacctactatccagacagtgtgaagggccgattcaccatctccagagacaatgccaagaacaccctgtacctgcaaatgagcagtctgaggtctgaggacacggccatatattactgtacaagagagggctactacggttcttttgactactggggccaaggcaccactctcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 21 - protein, 473 aa, synthetic construct
  REGION 1..473: note = Heavy chain amino acid sequence
MNFVLSLIFLALILKGVQCEVQLVESGGGSVKPGGSLKLSCAASGFTFSRYAMSWVRQTPEKRLEWVATINDGGSYTYYPDSVKGRFTISRDNAKNTLYLQMSSLRSEDTAIYYCTREGYYGSFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 22 - DNA, 717 bp, synthetic construct
  misc_feature 1..717: note = Anti-sIL7R mAB Clone ABS-2014 Light Chain DNA Sequence
  source 1..717: mol_type = other DNA
atgagtcctgcccagttcctgtttctgctagtgctctcgattcaggaaatcaacggtgatgttgtgatgacccagactccactcactttgtcggttaccattggacaaccagcttccatctcttgcaagtcaagtcagagcctcttatatactaatggaaaaacctatttgaattggttattacagaggccaggccagtctccaaaacgcctaatctatctggtgtctaaattggactctggagtccctgacaggttcagtggcagtggatcagggacagatttcacactgaaaatcagcagagtggaggctgaggatttgggagtttattactgcttgcagagtacacgttttcctcagacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccagagacatcaatgtcaagtggaagattgatggcagtgaacgacaaaatggtgtcctgaacagttggactgatcaggacagcaaagacagcacctacagcatgagcagcaccctcacattgaccaaggacgagtatgaacgacataacagctatacctgtgaggccactcacaagacatcaacttcacccatcgtcaagagcttcaacaggaatgagtgttag

SEQ ID NO: 23 - protein, 238 aa, synthetic construct
  REGION 1..238: note = Light chain amino acid sequence
MSPAQFLFLLVLSIQEINGDVVMTQTPLTLSVTIGQPASISCKSSQSLLYTNGKTYLNWLLQRPGQSPKRLIYLVSKLDSGVPDRFSGSGSGTDFTLKISRVEAEDLGVYYCLQSTRFPQTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 24 - DNA, 1413 bp, synthetic construct
  misc_feature 1..1413: note = Anti-sIL7R mAB Clone ABS-2102 Heavy Chain DNA Sequence
  source 1..1413: mol_type = other DNA
atgggatggaactggatctttattttaatcctgtcagtaactacaggtgtccactctgaggtccagctgcagcagtctggacctgagctggtgaagcctggggcttcagtgaagatatcctgcaaggcttctggttactcattcactggctactacatgcactgggtgaagcaaagtcctgaaaagagccttgagtggattggaaagattaatccttacactggtggtactacctacaaccacaagttcaaggccaaggccacattgactgtagacaaatcctccagcacagcctacatgcagctcaagagcctgacatctgaggactctgcagtctattactgtgcaagaagttattactacgctggtagccccccctttgactactggggccaaggcaccactctcacagtctcctcagccaaaacaacagccccatcggtctatccactggcccctgtgtgtggagatacaagtggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 25 - protein, 470 aa, synthetic construct
  REGION 1..470: note = Heavy chain amino acid sequence
MGWNWIFILILSVTTGVHSEVQLQQSGPELVKPGASVKISCKASGYSFTGYYMHWVKQSPEKSLEWIGKINPYTGGTTYNHKFKAKATLTVDKSSSTAYMQLKSLTSEDSAVYYCARSYYYAGSPPFDYWGQGTTLTVSSAKTTAPSVYPLAPVCGDTSGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - DNA, 708 bp, synthetic construct
  misc_feature 1..708: note = Anti-sIL7R mAB Clone ABS-2102 Light Chain DNA Sequence
  source 1..708: mol_type = other DNA
atggattttcaggtgcagattttcagcttcctgctaatcagtgcctcagtcataatgtccagaggagaaaatgtgctcacccagtctccagcaatcatgtctgcaactctaggggagaaggtcaccatgagctgcagggccagctcaaatgtaaagtacatgtactggtaccagcagaagtcaggtgcctcccccaaactatggatttattacacatccaacctggcttctggagtcccaactcgcttcagtggcagtgggtctgggacctcttattctctcacactcagcagcgtggaggctgaagatgctgccacttattactgccagcagtttactagttccccatggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgttag

SEQ ID NO: 27 - protein, 235 aa, synthetic construct
  REGION 1..235: note = Light chain amino acid sequence
MDFQVQIFSFLLISASVIMSRGENVLTQSPAIMSATLGEKVTMSCRASSNVKYMYWYQQKSGASPKLWIYYTSNLASGVPTRFSGSGSGTSYSLTLSSVEAEDAATYYCQQFTSSPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 28 - DNA, 1422 bp, synthetic construct
  misc_feature 1..1422: note = Anti-sIL7R mAB Clone ABS-2022 Heavy Chain DNA Sequence
  source 1..1422: mol_type = other DNA
atgggttggctgtggaacttgctattcctgatggcagctgcccaaagtgcccaagcacagatccagttggtacagtctggacctgagctgaagaagcctggagagacagtcaagatctcctgcaaggcttctgggtataccttcacaacctatggaatgaactgggtgaaacaggctccaggaaagggtttaaagtggatgggctggataaacacctactctggagtgccaacatatgctgatgacttcaagggacggtttgccttctctttggaaacctctgccagcactgcctatttgcagatcaacaacctcaaaaatgaggacacggctacatatttctgtacaagatctgggggattacgaccctggtttgcttactggggccaagggactctggtcactgtctctgcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 29 - protein, 473 aa, synthetic construct
  REGION 1..473: note = Heavy chain amino acid sequence
MGWLWNLLFLMAAAQSAQAQIQLVQSGPELKKPGETVKISCKASGYTFTTYGMNWVKQAPGKGLKWMGWINTYSGVPTYADDFKGRFAFSLETSASTAYLQINNLKNEDTATYFCTRSGGLRPWFAYWGQGTLVTVSAAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 30 - DNA, 717 bp, synthetic construct
  misc_feature 1..717: note = Anti-sIL7R mAB Clone ABS-2022 Light Chain DNA Sequence
  source 1..717: mol_type = other DNA
atgagtcctgcccagttcctgtttctgctagtgctctcgattcaggaaaccaatggtgatgttgtgatgacccagactccactgtctttgtcggttaccattggacaaccagcctccatctcttgcaagtcaagtcagagcctcttacatagtaatggaaagacatatttgaattggttattacagaggccaggccagtctccaaagctcctaatctatctggtgtctaaactggaatctggcatccctgacaggttcagtggcagtggatcagggacagatttcacactgaaaatcagcagagtggaggttgaggatttgggagtttattcctgcttgcaacatacacattttcctcatacgttcggatcggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgttag

SEQ ID NO: 31 - protein, 238 aa, synthetic construct
  REGION 1..238: note = Light chain amino acid sequence
MSPAQFLFLLVLSIQETNGDVVMTQTPLSLSVTIGQPASISCKSSQSLLHSNGKTYLNWLLQRPGQSPKLLIYLVSKLESGIPDRFSGSGSGTDFTLKISRVEVEDLGVYSCLQHTHFPHTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - DNA, 1428 bp, synthetic construct
  misc_feature 1..1428: note = Anti-sIL7R mAB Clone ABS-2018 Heavy Chain DNA Sequence
  source 1..1428: mol_type = other DNA
atggaatggatctggatctttctcttcatcctgtcaggaactgcaggtgtccaatcccaggttcagctgcagcagtctggagctgagctggcgaggcctggggcttcagtgaagctgtcctgcaaggcttctggctacaccttcacaagctatggtataagctgggtgaagcagagaactggacagggccttgagtggtttggagagatttatcctagaagtggtaatacttactacaatgagaagttcaaggtcaaggccacactgactgcagacaagtcctccagcacagcgtacatggagctccgcagcctgacatctgaggactctgcggtctatttctgtgcaagatcagggccaccttattactatgctatggactactggggtcaaggaacctcagtcaccgtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 33 - protein, 475 aa, synthetic construct
  REGION 1..475: note = Heavy chain amino acid sequence
MEWIWIFLFILSGTAGVQSQVQLQQSGAELARPGASVKLSCKASGYTFTSYGISWVKQRTGQGLEWFGEIYPRSGNTYYNEKFKVKATLTADKSSSTAYMELRSLTSEDSAVYFCARSGPPYYYAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 34 - DNA, 717 bp, synthetic construct
  misc_feature 1..717: note = Anti-sIL7R mAB Clone ABS-2018 Light Chain DNA Sequence
  source 1..717: mol_type = other DNA
atgaagttgcctgttaggttgttggtgctgttgttctggattcctgcttccagcagtgatgttgtgatgacccaaactccactctccctgcctgtcagtcttggagatcaagcttccatctcttgcagatctagtcagagccttgtacacagcaatggaaacacctatttatattggtacctgcagaagccaggccagtctccaaaggtcctgatctacagggtttccaaccgattttctggggtcccagacaggttcagtggcagtggatcagggacagatttcacactcaagatcagcagagtggaggctgaggatctgggagtttatttctgctttcaaggtacacatgttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgttag

SEQ ID NO: 35 - protein, 238 aa, synthetic construct
  REGION 1..238: note = Light chain amino acid sequence
MKLPVRLLVLLFWIPASSSDVVMTQTPLSLPVSLGDQASISCRSSQSLVHSNGNTYLYWYLQKPGQSPKVLIYRVSNRFSGVPDRFSGSGSGTDFTLKISRVEAEDLGVYFCFQGTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 36 - DNA, 1422 bp, synthetic construct; identical to SEQ ID NO: 28
  misc_feature 1..1422: note = Anti-sIL7R mAB Clone ABS-2028 Heavy Chain DNA Sequence
  source 1..1422: mol_type = other DNA

SEQ ID NO: 37 - protein, 473 aa, synthetic construct; identical to SEQ ID NO: 29
  REGION 1..473: note = Heavy chain amino acid sequence

SEQ ID NO: 38 - DNA, 717 bp, synthetic construct; identical to SEQ ID NO: 30
  misc_feature 1..717: note = Anti-sIL7R mAB Clone ABS-2028 Light Chain DNA Sequence
  source 1..717: mol_type = other DNA

SEQ ID NO: 39 - protein, 238 aa, synthetic construct; identical to SEQ ID NO: 31
  REGION 1..238: note = Light chain amino acid sequence

SEQ ID NO: 40 - DNA, 1425 bp, synthetic construct
  misc_feature 1..1425: note = Anti-sIL7R mAB Clone ABS-2045 Heavy Chain DNA Sequence
  source 1..1425: mol_type = other DNA
atgaactttgtgctcagcttgattttccttgccctcattttaaaaggtgtccagtgtgaagtgcagctggtggagtctgggggaggcttagtgaagcctggagggtccctaaaactctcctgtgctgcctctggattcactttcagtagctatgccatgtcttgggttcgccagactccggagaagaggctggagtgggtcgcaagcattagtagtggtggtagttacacctactatccagacagtgtgaagggtcgattcaccatctccagagacaatgccaagaacaccctgtacctgcaaatgagcagtctgaggtctgaggacacggccatgtattactgtgcaagactgggcaccgtttactatgctatggactactggggtcaaggaacctcagtcaccgtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 41 - protein, 474 aa, synthetic construct
  REGION 1..474: note = Heavy chain amino acid sequence
MNFVLSLIFLALILKGVQCEVQLVESGGGLVKPGGSLKLSCAASGFTFSSYAMSWVRQTPEKRLEWVASISSGGSYTYYPDSVKGRFTISRDNAKNTLYLQMSSLRSEDTAMYYCARLGTVYYAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 42 - DNA, 705 bp, synthetic construct
  misc_feature 1..705: note = Anti-sIL7R mAB Clone ABS-2045 Light Chain DNA Sequence
  source 1..705: mol_type = other DNA
atggattttcaggtgcagattttcagcttcctgctaatcagtgcctcagtcataatgtccagaggacaaattgttctcacccagtctccagcaatcatgtctgcatctctaggggaggagatcaccctaacctgcagtgccagctcgagtgtaagttacatgcactggtaccagcagaagtcaggcacttctcccaaactcttgatttatagcacatccaacctggcttctggagtcccttctcgcttcagtggcagtgggtctgggaccttttattctctcacaatcagcagtgtggaggctgaagatgctgccgattattactgccatcagtggagtagttatcggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 43 - protein, 234 aa, synthetic construct
  REGION 1..234: note = Light chain amino acid sequence
MDFQVQIFSFLLISASVIMSRGQIVLTQSPAIMSASLGEEITLTCSASSSVSYMHWYQQKSGTSPKLLIYSTSNLASGVPSRFSGSGSGTFYSLTISSVEAEDAADYYCHQWSSYRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 44 - protein, 5 aa, synthetic construct
  REGION 1..5: note = Heavy chain CDR
TYGMS

SEQ ID NO: 45 - protein, 17 aa, synthetic construct
  REGION 1..17: note = Heavy chain CDR
CINTYSGVPTYPDDFKG

SEQ ID NO: 46 - protein, 13 aa, synthetic construct
  REGION 1..13: note = Heavy chain CDR
EKNYYGARYYFDF

SEQ ID NO: 47 - protein, 16 aa, synthetic construct
  REGION 1..16: note = Light chain CDR
RSSQSIVHSYRYTYLE

SEQ ID NO: 48 - protein, 7 aa, synthetic construct
  REGION 1..7: note = Light chain CDR
GVSNRFS

SEQ ID NO: 49 - protein, 9 aa, synthetic construct
  REGION 1..9: note = Light chain CDR
FQGTHVPLT

SEQ ID NO: 50 - protein, 7 aa, synthetic construct
  REGION 1..7: note = Heavy chain CDR
TYGMGVG

SEQ ID NO: 51 - protein, 16 aa, synthetic construct
  REGION 1..16: note = Heavy chain CDR
NIWWNDYKYYNSALKS

SEQ ID NO: 52 - protein, 9 aa, synthetic construct
  REGION 1..9: note = Heavy chain CDR
VGNYDGGFH

SEQ ID NO: 53 - protein, 16 aa, synthetic construct
  REGION 1..16: note = Light chain CDR
RSSQTLVHSNGNTYLH

SEQ ID NO: 54 - protein, 7 aa, synthetic construct
  REGION 1..7: note = Light chain CDR
KVSNRFS

SEQ ID NO: 55 - protein, 9 aa, synthetic construct
  REGION 1..9: note = Light chain CDR
SQSTHVPWT

SEQ ID NO: 56 - protein, 5 aa, synthetic construct
  REGION 1..5: note = Heavy chain CDR
TYGMN

SEQ ID NO: 57 - protein, 17 aa, synthetic construct
  REGION 1..17: note = Heavy chain CDR
WINTYSGVPTYVDDFKG

SEQ ID NO: 58 - protein, 10 aa, synthetic construct
  REGION 1..10: note = Heavy chain CDR
WGTPRYFFDY

SEQ ID NO: 59 - protein, 16 aa, synthetic construct
  REGION 1..16: note = Light chain CDR
RSSQSIVHSNRYTYLE

SEQ ID NO: 60 - protein, 7 aa, synthetic construct
  REGION 1..7: note = Light chain CDR
GVSNRFS

SEQ ID NO: 61 - protein, 9 aa, synthetic construct
  REGION 1..9: note = Light chain CDR
FQGTHVPWT

SEQ ID NO: 62 - protein, 5 aa, synthetic construct
  REGION 1..5: note = Heavy chain CDR
TYGMN

SEQ ID NO: 63 - protein, 17 aa, synthetic construct
  REGION 1..17: note = Heavy chain CDR
WINTYSGVPTYADDFKG

SEQ ID NO: 64 - protein, 10 aa, synthetic construct
  REGION 1..10: note = Heavy chain CDR
SGGLRPWFAY

SEQ ID NO: 65 - protein, 16 aa, synthetic construct
  REGION 1..16: note = Light chain CDR
KSSQSLLHSNGKTYLN

SEQ ID NO: 66 - protein, 7 aa, synthetic construct
  REGION 1..7: note = Light chain CDR
LVSKLES

SEQ ID NO: 67 - protein, 9 aa, synthetic construct
  REGION 1..9: note = Light chain CDR
LQHTHFPHT

SEQ ID NO: 68 - protein, 5 aa, synthetic construct
  REGION 1..5: note = Heavy chain CDR
RYAMS

SEQ ID NO: 69 - protein, 17 aa, synthetic construct
  REGION 1..17: note = Heavy chain CDR
TINDGGSYTYYPDSVKG

SEQ ID NO: 70 - protein, 9 aa, synthetic construct
  REGION 1..9: note = Heavy chain CDR
EGYYGSFDY

SEQ ID NO: 71 - protein, 16 aa, synthetic construct
  REGION 1..16: note = Light chain CDR
KSSQSLLYTNGKTYLN

SEQ ID NO: 72 - protein, 7 aa, synthetic construct
  REGION 1..7: note = Light chain CDR
LVSKLDS

SEQ ID NO: 73 - protein, 9 aa, synthetic construct
  REGION 1..9: note = Light chain CDR
LQSTRFPQT

SEQ ID NO: 74 - protein, 5 aa, synthetic construct
  REGION 1..5: note = Heavy chain CDR
GYYMH

SEQ ID NO: 75 - protein, 17 aa, synthetic construct
  REGION 1..17: note = Heavy chain CDR
KINPYTGGTTYNHKFKA

SEQ ID NO: 76 - protein, 12 aa, synthetic construct
  REGION 1..12: note = Heavy chain CDR
SYYYAGSPPFDY

SEQ ID NO: 77 - protein, 10 aa, synthetic construct
  REGION 1..10: note = Light chain CDR
RASSNVKYMY

SEQ ID NO: 78 - protein, 7 aa, synthetic construct
  REGION 1..7: note = Light chain CDR
YTSNLAS

SEQ ID NO: 79 - protein, 9 aa, synthetic construct
  REGION 1..9: note = Light chain CDR
QQFTSSPWT

SEQ ID NO: 80 - protein, 5 aa, synthetic construct
  REGION 1..5: note = Heavy chain CDR
TYGMN

SEQ ID NO: 81 - protein, 17 aa, synthetic construct
  REGION 1..17: note = Heavy chain CDR
WINTYSGVPTYADDFKG

SEQ ID NO: 82 - protein, 10 aa, synthetic construct
  REGION 1..10: note = Heavy chain CDR
SGGLRPWFAY

SEQ ID NO: 83 - protein, 16 aa, synthetic construct
  REGION 1..16: note = Light chain CDR
KSSQSLLHSNGKTYLN

SEQ ID NO: 84 - protein, 7 aa, synthetic construct
  REGION 1..7: note = Light chain CDR
LVSKLES

SEQ ID NO: 85 - protein, 9 aa, synthetic construct
  REGION 1..9: note = Light chain CDR
LQHTHFPHT

SEQ ID NO: 86 - protein, 5 aa, synthetic construct
  REGION 1..5: note = Heavy chain CDR
SYGIS

SEQ ID NO: 87 - protein, 17 aa, synthetic construct
  REGION 1..17: note = Heavy chain CDR
EIYPRSGNTYYNEKFKV

SEQ ID NO: 88 - protein, 11 aa, synthetic construct
  REGION 1..11: note = Heavy chain CDR
SGPPYYYAMDY

SEQ ID NO: 89 - protein, 16 aa, synthetic construct
  REGION 1..16: note = Light chain CDR
RSSQSLVHSNGNTYLY

SEQ ID NO: 90 - protein, 7 aa, synthetic construct
  REGION 1..7: note = Light chain CDR
RVSNRFS

SEQ ID NO: 91 - protein, 9 aa, synthetic construct
  REGION 1..9: note = Light chain CDR
FQGTHVPWT

SEQ ID NO: 92 - protein, 5 aa, synthetic construct
  REGION 1..5: note = Heavy chain CDR
TYGMN

SEQ ID NO: 93 - protein, 17 aa, synthetic construct
  REGION 1..17: note = Heavy chain CDR
WINTYSGVPTYADDFKG

SEQ ID NO: 94 - protein, 10 aa, synthetic construct
  REGION 1..10: note = Heavy chain CDR
SGGLRPWFAY

SEQ ID NO: 95 - protein, 16 aa, synthetic construct
  REGION 1..16: note = Light chain CDR
KSSQSLLHSNGKTYLN

SEQ ID NO: 96 - protein, 7 aa, synthetic construct
  REGION 1..7: note = Light chain CDR
LVSKLES

SEQ ID NO: 97 - protein, 9 aa, synthetic construct
  REGION 1..9: note = Light chain CDR
LQHTHFPHT

SEQ ID NO: 98 - protein, 10 aa, synthetic construct
  REGION 1..10: note = Light chain CDR
SASSSVSYMH

SEQ ID NO: 99 - protein, 7 aa, synthetic construct
  REGION 1..7: note = Light chain CDR
STSNLAS

SEQ ID NO: 100 - protein, 8 aa, synthetic construct
  REGION 1..8: note = Light chain CDR
HQWSSYRT